SEQUENCE LISTING (7 sequences)

SEQ ID NO: 1 - protein, 889 aa, Homo sapiens
  IRP1
MSNPFAHLAEPLDPVQPGKKFFNLNKLEDSRYGRLPFSIRVLLEAAIRNCDEFLVKKQDIENILHWNVTQHKNIEVPFKPARVILQDFTGVPAVVDFAAMRDAVKKLGGDPEKINPVCPADLVIDHSIQVDFNRRADSLQKNQDLEFERNRERFEFLKWGSQAFHNMRIIPPGSGIIHQVNLEYLARVVFDQDGYYYPDSLVGTDSHTTMIDGLGILGWGVGGIEAEAVMLGQPISMVLPQVIGYRLMGKPHPLVTSTDIVLTITKHLRQVGVVGKFVEFFGPGVAQLSIADRATIANMCPEYGATAAFFPVDEVSITYLVQTGRDEEKLKYIKKYLQAVGMFRDFNDPSQDPDFTQVVELDLKTVVPCCSGPKRPQDKVAVSDMKKDFESCLGAKQGFKGFQVAPEHHNDHKTFIYDNTEFTLAHGSVVIAAITSCTNTSNPSVMLGAGLLAKKAVDAGLNVMPYIKTSLSPGSGVVTYYLQESGVMPYLSQLGFDVVGYGCMTCIGNSGPLPEPVVEAITQGDLVAVGVLSGNRNFEGRVHPNTRANYLASPPLVIAYAIAGTIRIDFEKEPLGVNAKGQQVFLKDIWPTRDEIQAVERQYVIPGMFKEVYQKIETVNESWNALATPSDKLFFWNSKSTYIKSPPFFENLTLDLQPPKSIVDAYVLLNLGDSVTTDHISPAGNIARNSPAARYLTNRGLTPREFNSYGSRRGNDAVMARGTFANIRLLNRFLNKQAPQTIHLPSGEILDVFDAAERYQQAGLPLIVLAGKEYGAGSSRDWAAKGPFLLGIKAVLAESYERIHRSNLVGMGVIPLEYLPGENADALGLTGQERYTIIIPENLKPQMKVQVKLDTGKTFQAVMRFDTDVELTYFLNGGILNYMIRKMAK

SEQ ID NO: 2 - protein, 963 aa, Homo sapiens
  IRP2 isoform 1
MDAPKAGYAFEYLIETLNDSSHKKFFDVSKLGTKYDVLPYSIRVLLEAAVRNCDGFLMKKEDVMNILDWKTKQSNVEVPFFPARVLLQDFTGIPAMVDFAAMREAVKTLGGDPEKVHPACPTDLTVDHSLQIDFSKCAIQNAPNPGGGDLQKAGKLSPLKVQPKKLPCRGQTTCRGSCDSGELGRNSGTFSSQIENTPILCPFHLQPVPEPETVLKNQEVEFGRNRERLQFFKWSSRVFKNVAVIPPGTGMAHQINLEYLSRVVFEEKDLLFPDSVVGTDSHITMVNGLGILGWGVGGIETEAVMLGLPVSLTLPEVVGCELTGSSNPFVTSIDVVLGITKHLRQVGVAGKFVEFFGSGVSQLSIVDRTTIANMCPEYGAILSFFPVDNVTLKHLEHTGFSKAKLESMETYLKAVKLFRNDQNSSGEPEYSQVIQINLNSIVPSVSGPKRPQDRVAVTDMKSDFQACLNEKVGFKGFQIAAEKQKDIVSIHYEGSEYKLSHGSVVIAAVISCTNNCNPSVMLAAGLLAKKAVEAGLRVKPYIRTSLSPGSGMVTHYLSSSGVLPYLSKLGFEIVGYGCSICVGNTAPLSDAVLNAVKQGDLVTCGILSGNKNFEGRLCDCVRANYLASPPLVVAYAIAGTVNIDFQTEPLGTDPTGKNIYLHDIWPSREEVHRVEEEHVILSMFKALKDKIEMGNKRWNSLEAPDSVLFPWDLKSTYIRCPSFFDKLTKEPIALQAIENAHVLLYLGDSVTTDHISPAGSIARNSAAAKYLTNRGLTPREFNSYGARRGNDAVMTRGTFANIKLFNKFIGKPAPKTIHFPSGQTLDVFEAAELYQKEGIPLIILAGKKYGSGNSRDWAAKGPYLLGVKAVLAESYEKIHKDHLIGIGIAPLQFLPGENADSLGLSGRETFSLTFPEELSPGITLNIQTSTGKVFSVIASFEDDVEITLYKHGGLLNFVARKFS

SEQ ID NO: 3 - protein, 713 aa, Homo sapiens
  IRP2 isoform 2
MAHQINLEYLSRVVFEEKDLLFPDSVVGTDSHITMVNGLGILGWGVGGIETEAVMLGLPVSLTLPEVVGCELTGSSNPFVTSIDVVLGITKHLRQVGVAGKFVEFFGSGVSQLSIVDRTTIANMCPEYGAILSFFPVDNVTLKHLEHTGFSKAKLESMETYLKAVKLFRNDQNSSGEPEYSQVIQINLNSIVPSVSGPKRPQDRVAVTDMKSDFQACLNEKVGFKGFQIAAEKQKDIVSIHYEGSEYKLSHGSVVIAAVISCTNNCNPSVMLAAGLLAKKAVEAGLRVKPYIRTSLSPGSGMVTHYLSSSGVLPYLSKLGFEIVGYGCSICVGNTAPLSDAVLNAVKQGDLVTCGILSGNKNFEGRLCDCVRANYLASPPLVVAYAIAGTVNIDFQTEPLGTDPTGKNIYLHDIWPSREEVHRVEEEHVILSMFKALKDKIEMGNKRWNSLEAPDSVLFPWDLKSTYIRCPSFFDKLTKEPIALQAIENAHVLLYLGDSVTTDHISPAGSIARNSAAAKYLTNRGLTPREFNSYGARRGNDAVMTRGTFANIKLFNKFIGKPAPKTIHFPSGQTLDVFEAAELYQKEGIPLIILAGKKYGSGNSRDWAAKGPYLLGVKAVLAESYEKIHKDHLIGIGIAPLQFLPGENADSLGLSGRETFSLTFPEELSPGITLNIQTSTGKVFSVIASFEDDVEITLYKHGGLLNFVARKFS

SEQ ID NO: 4 - protein, 906 aa, Homo sapiens
  IRP2 isoform 3 sequence 1
MKKEDVMNILDWKTKQSNVEVPFFPARVLLQDFTGIPAMVDFAAMREAVKTLGGDPEKVHPACPTDLTVDHSLQIDFSKCAIQNAPNPGGGDLQKAGKLSPLKVQPKKLPCRGQTTCRGSCDSGELGRNSGTFSSQIENTPILCPFHLQPVPEPETVLKNQEVEFGRNRERLQFFKWSSRVFKNVAVIPPGTGMAHQINLEYLSRVVFEEKDLLFPDSVVGTDSHITMVNGLGILGWGVGGIETEAVMLGLPVSLTLPEVVGCELTGSSNPFVTSIDVVLGITKHLRQVGVAGKFVEFFGSGVSQLSIVDRTTIANMCPEYGAILSFFPVDNVTLKHLEHTGFSKAKLESMETYLKAVKLFRNDQNSSGEPEYSQVIQINLNSIVPSVSGPKRPQDRVAVTDMKSDFQACLNEKVGFKGFQIAAEKQKDIVSIHYEGSEYKLSHGSVVIAAVISCTNNCNPSVMLAAGLLAKKAVEAGLRVKPYIRTSLSPGSGMVTHYLSSSGVLPYLSKLGFEIVGYGCSICVGNTAPLSDAVLNAVKQGDLVTCGILSGNKNFEGRLCDCVRANYLASPPLVVAYAIAGTVNIDFQTEPLGTDPTGKNIYLHDIWPSREEVHRVEEEHVILSMFKALKDKIEMGNKRWNSLEAPDSVLFPWDLKSTYIRCPSFFDKLTKEPIALQAIENAHVLLYLGDSVTTDHISPAGSIARNSAAAKYLTNRGLTPREFNSYGARRGNDAVMTRGTFANIKLFNKFIGKPAPKTIHFPSGQTLDVFEAAELYQKEGIPLIILAGKKYGSGNSRDWAAKGPYLLGVKAVLAESYEKIHKDHLIGIGIAPLQFLPGENADSLGLSGRETFSLTFPEELSPGITLNIQTSTGKVFSVIASFEDDVEITLYKHGGLLNFVARKFS

SEQ ID NO: 5 - protein, 906 aa, Homo sapiens
  IRP2 isoform 3 sequence 2
MKKEDVMNILDWKTKQSNVEVPFFPARVLLQDFTGIPAMVDFAAMREAVKTLGGDPEKVHPACPTDLTVDHSLQIDFSKCAIQNAPNPGGGDLQKAGKLSPLKVQPKKLPCRGQTTCRGSCDSGELGRNSGTFSSQIENTPILCPFHLQPVPEPETVLKNQEVEFGRNRERLQFFKWSSRVFKNVAVIPPGTGMAHQINLEYLSRVVFEEKDLLFPDSVVGTDSHITMVNGLGILGWGVGGIETEAVMLGLPVSLTLPEVVGCELTGSSNPFVTSIDVVLGITKHLRQVGVAGKFVEFFGSGVSQLSIVDRTTIANMCPEYGAILSFFPVDNVTLKHLEHTGFSKAKLESMETYLKAVKLFRNDQNSSGEPEYSQVIQINLNSIVPSVSGPKRPQDRVAVTDMKSDFQACLNEKVGFKGFQIAAEKQKDIVSIHYEGSEYKLSHGSVVIAAVISCTNNCNPSVMLAAGLLAKKAVEAGLRVKPYIRTSLSPGSGMVTHYLSSSGVLPYLSKLGFEIVGYGCSTCVGNTAPLSDAVLNAVKQGDLVTCGILSGNKNFEGRLCDCVRANYLASPPLVVAYAIAGTVNIDFQTEPLGTDPTGKNIYLHDIWPSREEVHRVEEEHVILSMFKALKDKIEMGNKRWNSLEAPDSVLFPWDLKSTYIRCPSFFDKLTKEPIALQAIENAHVLLYLGDSVTTDHISPAGSIARNSAAAKYLTNRGLTPREFNSYGARRGNDAVMTRGTFANIKLFNKFIGKPAPKTIHFPSGQTLDVFEAAELYQKEGIPLIILAGKKYGSGNSRDWAAKGPYLLGVKAVLAESYEKIHKDHLIGIGIAPLQFLPGENADSLGLSGRETFSLTFPEELSPGITLNIQTSTGKVFSVIASFEDDVEITLYKHGGLLNFVARKFS

SEQ ID NO: 6 - protein, 343 aa, Homo sapiens
  IRP2 isoform 4
MDAPKAGYAFEYLIETLNDSSHKKFFDVSKLGTKYDVLPYSIRVLLEAAVRNCDGFLMKKEDVMNILDWKTKQSNVEVPFFPARVLLQDFTGIPAMVDFAAMREAVKTLGGDPEKVHPACPTDLTVDHSLQIDFSKCAIQNAPNPGGGDLQKAGKLSPLKVQPKKLPCRGQTTCRGSCDSGELGRNSGTFSSQIENTPILCPFHLQPVPEPETVLKNQEVEFGRNRERLQFFKWSSRVFKNVAVIPPGTGMAHQINLEYLSRVVFEEKDLLFPDSVVGTDSHITMVNGLGILGWGVGGIETEAVMLGLPVSLTLPEVVGCELTGSSNPFVTSIDVVLGITKVS

SEQ ID NO: 7 - protein, 18 aa, Artificial Sequence
  T2A peptide
EGRGSLLTCGDVEENPGP

(19) United States
(12) Patent Application Publication
(10) Pub. No.: US 2022/0306989 A1
(43) Pub. Date: Sep. 29, 2022

(54) CELL THERAPY METHODS

(71) Applicant: Universität Basel, Basel (CH)

(72) Inventors: Jasmin GRÄHLERT, Basel (CH); Christoph HESS, Basel (CH)

(21) Appl. No.: 17/635,534

(22) PCT Filed: Aug. 19, 2020
(86) PCT No.: PCT/EP2020/073195

§ 371 Date: Feb. 15, 2022

(30) Foreign Application Priority Data
Aug. 19, 2019 (EP) 19192299.6

(51) Int. Cl. C12N 5/0783; C07K 14/725; C07K 16/30; C12N 15/86; A61P 35/00

(52) U.S. Cl. CPC C12N 5/0636; C07K 14/7051; C12N 5/0646; C07K 16/30; C12N 15/86; A61P 35/00; C07K 2319/03; C12N 2510/00; C07K 2319/33; C12N 2500/24

(57) ABSTRACT

The present invention is in the field of cell therapy and provides compositions and methods for treating cancer and/or viral infections in patients. The invention provides lymphocytes comprising a synthetic polynucleotide encoding at least one iron regulatory protein and, optionally, a chimeric antigen receptor. The invention further provides methods for producing these lymphocytes and administering them to patients.

The present invention is in the field of cell therapy and provides compositions and methods for treating cancer and/or viral infections in patients. The invention provides lymphocytes comprising a synthetic polynucleotide encoding at least one iron regulatory protein and, optionally, a chimeric antigen receptor. The invention further provides methods for producing these lymphocytes and administering them to patients.

### INTRODUCTION

In the past few decades, the potency of the immune system in the development and treatment of cancer has been a major focal point of research. Although targeted therapy and immunotherapy with immune checkpoint blockade have greatly improved the survival of many cancer patients, a large proportion of patients still develop disease progression upon these therapies. Adoptive cell therapy (ACT) may provide an additional treatment option for these patients and comprises the intravenous transfer of either tumor-resident or peripheral blood modified immune cells into cancer patients to mediate an anti-tumor function. Currently, ACT can be classified into three different types with each their own mechanism of action, namely ACT with tumor-infiltrating lymphocytes (TIL), ACT using T cell receptor (TCR) gene therapy, and ACT with chimeric antigen receptor (CAR) modified T cells. The use of other immune cell types such as natural killer cells as a basis for cell therapy is also an area of current research.

The first studies with TIL were performed by Rosenberg and coworkers at the Surgery Branch in the National Institutes of Health (SB, NIH, Bethesda, Md., US), where TIL were grown from different murine tumors and showed anti-tumor activity in vivo. The current TIL therapy consists of ex vivo expansion of TIL from resected tumor material and adoptive transfer into the patient following a lymphodepleting preparative regimen and subsequent support of interleukin-2 (IL-2). With this regimen, remarkable objective tumor responses of around 50% have been achieved in patients with metastatic melanoma in several phase I/II clinical trials. After the successes seen with TIL in melanoma patients, the production of TIL from other solid tumor types has also been studied. Up until now, it has been possible to grow out TILs from non-melanoma tumor types such cervical cancer, renal cell cancer, breast cancer, and non-small cell lung cancer with varying rates of tumor reactivity.

Next to the naturally occurring TILs in tumors and the thereupon-based treatment options, peripheral blood T cells can be isolated and genetically modified in vitro to express TCRs that target specific tumor antigens for the use of ACT. With the use of this method, large pools of tumor specific T cells can be generated, with potent anti-tumor activity and objective clinical responses observed in up to 30% of treated patients. For the recognition by the modified TCR, antigen presentation via the major histocompatibility complex (MHC) is required. However, it is well known that many cancer types can escape T cell-mediated immune responses by downregulation or loss of their MHC expression. To circumvent the need for the presence of MHC on tumor cells for the recognition by tumor-specific T cells, artificial receptors such as CAR molecules have been developed. ACT with CAR-modified T cells holds the capacity of the same effector function as TCR-modified T cells, but independently of MHC expression. Besides the use of protein antigens, other antigens such as carbohydrates or glycolipid antigens have also been explored. Impressive clinical responses have already been seen in hematological malignancies with CD19-specific CAR T cells, which led to the exploration of using CAR therapy in solid tumors as well.

While hematopoietic stem cell transplant (HSCT) offers a chance of cure for patients with many high risk cancers or primary immunodeficiency syndromes, transplant recipients remain vulnerable to infectious complications due to prolonged and profound immunosuppression. These risks are modified by preparative regimen, transplant type, and duration of myelosuppression. With advances in conditioning regimens and improved post-transplant management, an increasing number of patients are eligible to receive mismatched, unrelated, or haploidentical donor HSCT. While there have been great improvements in outcome for patients with severe or otherwise untreatable disease, the immunosuppression required for engraftment and, when indicated, to treat graft versus host disease (GVHD), opens the door for infection. In particular, viral infections cause significant morbidity and mortality, and the risk increases when T cell immune reconstitution is delayed. The relationship between immunosuppression, immune reconstitution, and the effects of GVHD, and infection are complicated and intertwined. Pharmacologic treatment and prophylactic options for viral infections remain limited and often ineffective, with associated morbidities notably from acute kidney injury and myelosuppression. Treatment may also generate resistance, and does not confer extended protection leaving patients at risk for viral reactivation. Given the correlation between delay in T cell immune recovery and viral disease, adoptive cell therapy is a logical alternative to pharmacologic therapy. Unmanipulated lymphocyte infusions from seropositive donors have been infused in patients with life-threatening disease such as EBV-associated lymphoma, demonstrating clinical efficacy with risks primarily associated with GVHD. This strategy has evolved over the past two decades, and donor lymphocyte products have been successful in reconstituting viral immunity in the host as a treatment for viral disease (including reactivation, new exposure, and lymphoma) and as prophylaxis. Following these initial studies, virus-specific T cell (VST) selection and/or expansion has been refined to maximize viral cytotoxicity and minimize alloreactivity to reduce and largely eliminate the risk of GVHD. In current studies, VSTs offer targeted therapy and have demonstrated a very good safety profile to date.

Natural killer cells (NK cells) have been studies to a lesser extent for their potential in ACT compared to T cells. However, several attributes of NK cells make them ideal candidates for adoptive cell therapy. In addition to being highly cytotoxic effectors, NK cells are not restricted by antigen specificity, and they rapidly produce proinflammatory cytokines that potentiate adaptive immune responses.

NK cells from patients with cancer are often dysfunctional, displaying reduced rates of proliferation, decreased responses to cytokine stimulation and reduced effector function. Thus, early immunotherapeutic strategies aimed to enhance or restore functions of endogenous NK cells. These strategies involved IL-2-induced activation of autologous NK cells ex vivo, followed by reinfusion into the patient together with combined IL-2 treatment during the course of therapy. Unfortunately, IL-2-activated NK cells did not impact tumor growth and the treatment regimen had severe side effects. The use of allogeneic NK cells for the treatment of cancer patients is more promising, since allogeneic NK cells are fully functional, as compared to patients NK cells. In addition, allogeneic NK cells have graft-versus-leukemia/tumor (GvL/GvT) effects without causing graft-versus-host disease (GvHD), thus cause less immunopathology.

Clinically relevant responses have been achieved, particularly in the treatment of hematologic malignancies, such as acute myeloid lymphoma (AML) and non-Hodgkin lymphoma (NHL). However, the activity of NK cells alone is often insufficient to fully control tumor growth; and the treatment of solid tumors is particularly challenging due to the restrictive tumor microenvironment. Thus, strategies to enhance NK cell function have been investigated extensively. One approach to enhance NK cell anti-tumor activity is the utilization of cytokines. Various cytokines have been used (IL-2, IL-12, IL-15, IL-18, IL-21 and type I interferons) for in vitro expansion and activation of NK cells prior to adoptive transfer.

One promising cytokine combination to maximize NK cell function is the combinatorial use of IL-12, IL-18 and IL-15. Stimulation with this combination induces a population of NK cells with “memory-like” features, such as prolonged survival and enhanced effector functions. Preclinical studies have shown that cytokine-enhanced (CE) NK cells have substantial potential as anti-leukemia cellular therapy. In in vivo tumor models of lymphoma or melanoma, CE NK cells had enhanced effector function (IFN-γ production and cytotoxicity). Also, following adoptive transfer into imnunodeficient NOD-SCID-γc −/− mice (NSG), IL-2-enhanced CE NK cells persisted longer compared to control NK cells. Finally, in AML xenografted NSG mice, CE NK cells substantially reduced AML burden and improved overall survival.

Molecular mechanisms driving increased effector functions of CE NK cells are currently unknown. For T cells, it is well established that the function of a certain subset, e.g. naive CD8+ T cells vs. memory CD8+ T cell, is linked to different mechanism of metabolic regulation. Thus, altered metabolic patterns in CE NK cells could support the enhanced function. Elucidating the molecular mechanisms that underpin differentiation of CE NK cells and their superior effector functionality is an important prerequisite to improve clinical efficacy.

In general, highly proliferating cells strictly depend on iron to support basic processes, such as energy metabolism/respiration, DNA synthesis and repair, and cell cycle control. The high amount of iron needed by proliferating cells, including lymphocytes, is provided by transferrin, which is taken up via the cell surface receptor CD71. CD71 is commonly used as a lymphocyte activation marker and is expressed on activated NK cells. Only few studies to date have investigated the importance of iron metabolism on lymphocyte function. A mutation in the CD71 receptor (TFRCY20H/Y20H) has recently been shown to impair T and B cell function due to impaired proliferation.

Reduced iron levels have been proposed to compromise NK cell cytotoxicity and dysfunctional NK cells and, in this setting, may contribute to cancer development in rats. In addition, low serum ferritin levels have been associated with reduced NK cell activity in humans. However, the specific impact of iron on NK cell-mediated immunity remains elusive.

Cellular iron homeostasis is a tightly regulated process that involves the coordination of iron uptake, utilization and storage. It is mainly regulated at the post-transcriptional level by the iron regulatory protein/iron-responsive element (IRP/IRE) regulatory system. IRP1 and IRP2 are RNA binding proteins that recognize IREs in distinct mRNAs, thereby controlling their stability and translation to protein. The activities of IRP1 and IRP2 are regulated in response to cellular iron levels. Canonical IREs are present in the 5′UTR or the 3′UTR of mRNAs encoding for iron acquisition, iron storage, iron utilization, ATP production and iron export.

Under iron-deficient conditions, the IRP activity is high and IRPs bind to IREs in the 5′ or 3′UTR of the corresponding mRNAs. Depending on the localization of the IRE, IRPs can differently impact protein expression. Translation of mRNAs harboring an IRE in the 5′UTR (e.g. FTH1 mRNA, ferritin light chain 1) is inhibited by IRP binding. In contrast, binding of IRPs to the 3′UTR IREs (e.g. TFRC mRNA, CD71) stabilizes mRNA and results in enhanced translation. Thus, the IRP/IRE regulatory network coordinates cellular iron homeostasis by selectively regulating the translation of certain mRNAs relative to the cellular iron status.

Despite the recent success in adoptive cell therapy, there is still a need for improving these therapies to make them available for more patients. One general requirement of successful adoptive cell therapies is to ensure that the immune cells robustly expand within the patient after the cells have been infused. This would, on the one hand result, in a reduced number of infusions and, on the other hand, result in a higher efficacy of the therapy. Thus, there is a need in the art for improved means and methods related to cell therapy. More particularly, there is a need for immune cells that robustly expand in vivo after the cells have been administered to a subject.

The technical problem is solved by the embodiments provided in the claims. That is, the present invention relates to the following items:

1. A lymphocyte comprising a synthetic polynucleotide encoding at least one iron regulatory protein.
2. The lymphocyte according to item 1, wherein the lymphocyte is a T cell or a natural killer cell.
3. The lymphocyte according to any one of items 1 or 2, wherein the at least one iron regulatory protein is constitutively expressed.
4. The lymphocyte according to any one of items 1 to 3, wherein the at least one iron regulatory protein is IRP1 (SEQ ID NO: 1) and/or IRP2 (SEQ ID NO: 2-6).
5. The lymphocyte according to any one of items 1 to 4, wherein the lymphocyte further comprises a chimeric antigen receptor.
6. The lymphocyte according to item 5, wherein the chimeric antigen receptor comprises an antigen binding domain, a transmembrane domain, a co-stimulatory signaling region and a signaling domain.
7. The lymphocyte according to item 6, wherein the antigen binding domain is an antibody or an antigen-binding fragment thereof, in particular wherein the antigen-binding fragment is a Fab or an scFv.
8. The lymphocyte according to any one of items 6 or 7, wherein the antigen binding domain specifically binds a tumor antigen.
9. The lymphocyte according to item 8, wherein the tumor antigen is present on the surface of cells of a target cell population or tissue.
10. A pharmaceutical composition comprising the lymphocyte according to any one of items 1 to 9 and a pharmaceutically acceptable carrier.
11. The lymphocyte according to any one of items 1 to 9 or the pharmaceutical composition according to item 10 for use in therapy.
12. The lymphocyte according to any one of items 1 to 9 or the pharmaceutical composition according to item 10 for use in treating cancer.
13. The lymphocyte or pharmaceutical composition for use according to item 12, wherein the cancer is a hematologic cancer or a solid tumor, in particular wherein the hematologic cancer is acute lymphoblastic leukemia, diffuse large B-cell lymphoma, Hodgkin's lymphoma, acute myeloid leukemia or multiple myeloma and wherein the solid tumor is colon cancer, breast cancer, pancreatic cancer, ovarian cancer, hepatocellular carcinoma, lung cancer, neuroblastoma, glioblastoma or sarcoma.
14. The lymphocyte according to any one of items 1 to 9 or the pharmaceutical composition according to item 10 for use in preventing and/or treating viral infections.
15. The lymphocyte or pharmaceutical composition for use according to item 14, wherein the viral infection is caused by a human immunodeficiency virus (HIV), adenoviruses, polyomaviruses, influenza virus or human herpesvirus, in particular wherein the human herpesvirus is cytomegalovirus (CMV), Epstein-Barr virus (EBV), herpes simplex virus (HSV), Varizella-Zoster virus (VZV) or human herpesvirus 8 (HHV8).
16. A method for treating a subject having cancer or for preventing and/or treating a viral infection in a subject, the method comprising administering to the subject a therapeutically effective amount of the lymphocyte according to any one of items 1 to 7 or the pharmaceutical composition according to item 10.
17. The method according to item 16, wherein the cancer is a hematologic cancer or a solid tumor, in particular wherein the hematologic cancer is acute lymphoblastic leukemia, diffuse large B-cell lymphoma, Hodgkin's lymphoma, acute myeloid leukemia or multiple myeloma and wherein the solid tumor is colon cancer, breast cancer, pancreatic cancer, ovarian cancer, hepatocellular carcinoma, lung cancer, neuroblastoma, glioblastoma or sarcoma.
18. The method according to item 16, wherein the viral infection is caused by human immunodeficiency virus (HIV), adenoviruses, polyomaviruses, influenza virus or human herpesvirus, in particular wherein the human herpesvirus is cytomegalovirus (CMV), Epstein-Barr virus (EBV), herpes simplex virus (HSV), Varizella-Zoster virus (VZV) or human herpesvirus 8 (HHV8).
19. A method for producing the lymphocyte according to any one of items 1 to 9, the method comprising the steps of:
a) providing a lymphocyte obtained from a subject;
b) introducing a synthetic polynucleotide encoding at least one iron regulatory protein into the lymphocyte; and
c) expressing the gene(s) encoded in the synthetic polynucleotide.
   20. The method according to item 19, wherein a second synthetic polynucleotide encoding a chimeric antigen receptor is introduced in step (b).
   21. The method according to item 20, wherein the synthetic polynucleotide encoding the chimeric antigen receptor is combined with the synthetic polynucleotide encoding the at least one iron regulatory protein.
   22. The method according to any one of items 19 to 21, wherein the lymphocyte is activated before or after the at least one synthetic polynucleotide is introduced into the lymphocyte.
   23. The method according to any one of items 19 to 22, wherein the at least one synthetic polynucleotide is introduced into the lymphocyte by viral transduction, in particular by retroviral transduction.

Accordingly, in one embodiment, the invention relates to a lymphocyte comprising a synthetic polynucleotide encoding at least one iron regulatory protein.

That is, the invention is based on the surprising finding that CD71-mediated iron uptake is a key metabolic checkpoint for activated NK cells, acting as a go/no-go gatekeeper with regard to cell proliferation. In cytokine-enhanced (CE) NK cells, surplus levels of iron regulatory proteins unexpectedly create a pseudo iron deficient state, thereby selectively enhancing translation of CD71 and thus increasing proliferation of the cells.

The molecular mechanisms that are responsible for the enhanced effector functions of CE NK cells remained unknown so far. Example 2 specifically shows that the expression of CD71 is significantly higher in CE NK cells in response to stimulation with IL-12 and IL-18 and tumor target cells, when compared to naïve (NV) NK cells (FIGS. 2B, 2C and 2D). In Example 5, it is further shown that the transcription of the TFRC gene, which encodes CD71, is induced upon activation of both NV and CE NK cells with cytokines, however, to a higher extent in CE NK cells (FIG. 5B). The higher TFRC mRNA expression directly translates into increased protein expression in CE NK cells compared to NV NK cells (FIGS. 2B and C). Accordingly, it is shown in Example 6, that the expression levels of the iron regulatory proteins (IRPs) IRP1 and IRP2 are higher in CE NK cells compared to NV NK cells (FIG. 6A). Since it is known that IRPs are involved in regulating the translation of the TFRC mRNA via stabilization of the latter, it can be concluded that the higher amounts of CD71 protein in CE NK compared to NV NK cells is caused by the higher amounts of IRPs in these cells. These findings are surprising, since it is known that the expression of IRPs is regulated in response to cellular iron levels. However, in CE NK cells, the expression of IRPs is upregulated despite abundant iron in the surrounding media, thereby creating a pseudo iron deficient state. Upon stimulation, this pseudo iron deficient state allows increased stabilization of CD71 mRNA resulting in increased CD71 protein expression and hence proliferation.

Based on these findings, the inventors have concluded that inducing a pseudo iron deficient state in lymphocytes, such as NK cells or T cells, results in increased proliferation, with, accordingly, a larger ensuing effector population after administration to a subject. Instead of necessarily treating lymphocytes with cytokines and/or feeder cell lines, which is often difficult or even impossible to control in in vivo applications, the present invention provides an inventive solution for inducing a pseudo iron deficient state in lymphocytes by overexpressing at least one IRP in said lymphocytes. Accordingly, the IRP-overexpressing, pseudo iron deficient lymphocyte according to the invention has enhanced functions, in particular enhanced proliferation, compared to a lymphocyte not overexpressing an IRP. Thus, in an alternative embodiment, the invention relates to a lymphocyte that overexpresses at least one iron-regulatory protein.

The inventors have demonstrated that enforced expression of IRPs results in increased proliferation of different types of lymphocytes. For example, it was shown by the inventors that lentiviral overexpression of IRP2 (SEQ ID NO:2; NCBI RefSeq: NM_004136.4) resulted in increased expression of CD71 and, more importantly, increased proliferation of Jurkat T cells (FIG. 8F). Further, it was shown by the inventors that lentiviral overexpression induced the expression of CD71 in CD4⁺ and CD8⁺ T cells (FIGS. 8G and H). Moreover, the inventors showed that lentiviral overexpression of IRP2 in CAR T cells resulted in increased proliferation of the CAR T cells upon stimulation with an antigen, while overexpression of IRP2 had no impact on the proliferation of an unstimulated CAR T cell (FIG. 8K).

Thus, the inventors convincingly demonstrated that the regulatory effects of IRPs on CD71 expression and the correlation between CD71 expression and cell proliferation are well conserved among different types of lymphocytes, in particular among T cells and NK cells. In view of these findings, it was concluded that overexpression of IRPs in lymphocytes results in increased proliferation of said lymphocytes. Thus, it is plausible that enforced overexpression of IRPs represents an attractive strategy for achieving robust in vivo proliferation of lymphocytes that have been infused into patients in the course of lymphocyte-based therapies.

IRPs, also known as iron-responsive element-binding proteins, are proteins that bind to iron-responsive elements (IREs) and thereby regulate human iron metabolism. In humans, two different IRPs, named IRP1 and IRP2, have been described. The activity of IRP1 vs. IRP2 is regulated in distinct ways. IRP1 contains an iron-sulfur cluster and under iron-replete conditions functions as cytosolic aconitase. When iron is scarce, the iron-sulfur cluster becomes devoid of iron and IRP1 changes its configuration, thus becoming able to bind to IREs of mRNAs. In contrast, IRP2 is rapidly degraded in relative iron excess by the ubiquitin proteasome system. Under iron-deplete conditions, the adaptor protein FBXL5 is degraded leading to increased IRP2 levels. Thus, the ubiquitin ligase functions as an iron sensor and a regulator of iron homeostasis. Tissue specific variation in activities of IRP1 and IRP2 have been described, and IRP1 and IRP2 knockout mice have distinct phenotypes.

The term “polynucleotide” as used herein refers to a sequence of nucleotides connected by phosphodiester linkages. A polynucleotide of this invention can be a deoxyribonucleic acid (DNA) molecule or ribonucleic acid (RNA) molecule in either single- or double-stranded form. Nucleotide bases are indicated herein by a single letter code: adenine (A), guanine (G), thymine (T), cytosine (C), inosine (I) and uracil (U). A polynucleotide of this invention may be prepared using standard techniques well known to one of ordinary skill in the art.

A “synthetic polynucleotide”, as used herein, is a polynucleotide that is of non-natural origin and has been integrated into a lymphocyte. Synthetic polynucleotides may be produced by recombinant techniques, including polymerase chain reaction, or by chemical synthesis. The skilled person is aware of methods to produce synthetic polynucleotides with a specific polynucleotide sequence. Further, the skilled person is aware of methods to integrate synthetic polynucleotides into a lymphocyte. Within the present invention, it is preferred that the synthetic polynucleotide comprises at least one gene or coding sequence encoding an IRP, wherein the at least one gene or coding sequence is operably linked to at least one regulatory element, for example a promoter, that is not naturally associated with the endogenous gene encoding the at least one IRP. Such a synthetic polynucleotide may be obtained by multiple strategies. For example, a polynucleotide comprising a gene or coding sequence encoding an IRP that is under control of a promoter or another regulatory element that is not naturally associated with the endogenous gene encoding the IRP may be integrated into the lymphocyte. Alternatively, a polynucleotide encoding an IRP may be integrated into the genome of a lymphocyte, such that it is operably linked to a regulatory element that is not naturally associated with the endogenous gene encoding the IRP. Further, the synthetic polynucleotide of the invention may also be obtained by integrating a polynucleotide comprising a regulatory element that is not naturally associated with the endogenous gene encoding the IRP into a lymphocyte, such that this regulatory element will be operably linked to the endogenous gene of the lymphocyte encoding the IRP. Alternatively, the synthetic polynucleotide of the invention may also be obtained by modifying the endogenous regulatory elements of a gene encoding an IRP by methods of genetic engineering or genome editing, such that the expression of the endogenous IRP gene of a lymphocyte is altered. For example, the regulatory elements, for example the promoter, of the endogenous gene encoding the IRP may be modified such that the gene encoding the IRP is constitutively expressed in the lymphocyte. Methods for genetic engineering are well known in the art and include CRISPR/Cas9, or the use of engineered nucleases such as meganucleases, zinc finger nucleases or TALENs.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame. The term “promoter” as used herein is defined as a DNA sequence recognized by the synthetic machinery of the cell, or introduced synthetic machinery, required to initiate the specific transcription of a polynucleotide sequence.

A cell, for example a lymphocyte, is said to comprise a synthetic polynucleotide, if the synthetic polynucleotide is present inside the cell, i.e. enclosed by the cytoplasmic membrane of the cell. The synthetic polynucleotide may be delivered into the cell in any form and by any method known in the art. For example, the synthetic polynucleotide may be present inside the cell as part of a circular DNA vector, e.g. a plasmid, in form or as part of a linear DNA or in form or as part of an mRNA. However, it is preferred that the synthetic polynucleotide is integrated as DNA into the genome of the lymphocyte.

The term “encoding” refers to the inherent property of specific sequences of nucleotides in a polynucleotide, such as a coding sequence, a gene, a cDNA, or an RNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a coding sequence or a gene encodes a protein if transcription of the coding sequence or the gene into mRNA and translation of mRNA corresponding to that coding sequence or gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that, coding sequence, gene or cDNA.

The term “coding sequence”, as used herein, refers to a nucleic acid sequence that is transcribed and translated into a polypeptide when placed under the control of appropriate regulatory or expression control sequences. The term “gene”, as used herein, refers to a DNA sequence, including but not limited to a DNA sequence that can be transcribed into mRNA which can be translated into polypeptide chains, transcribed into rRNA or tRNA or serve as recognition sites for enzymes and other proteins involved in DNA replication, transcription and regulation. The term “gene” is commonly understood to include all introns and other DNA sequences spliced from the mRNA transcript, along with variants resulting from alternative splice sites, when used in its endogenous context. However, when the term “gene” is used in the context of a synthetic polynucleotide, the term is broadly understood to further include coding sequences that correspond in sequence to spliced mRNA variants of a gene or cDNAs derived from a spliced mRNA of a gene.

The term “lymphocyte”, as used herein, refers to any of the mononuclear non-phagocytic leukocytes found in the blood, lymph, and lymphoid tissues which are derived from lymphoid stem cells; lymphocytes include natural killer cells (NK cells; which function in cell-mediated, cytotoxic innate immunity), T cells (for cell-mediated, cytotoxic adaptive immunity), and B cells (for humoral, antibody-driven adaptive immunity). Preferably, the lymphocyte of the present invention is an NK cell or a T cell. Thus, in a preferred embodiment, the invention relates to the lymphocyte according to the invention, wherein the lymphocyte is a T cell or a natural killer cell.

A T cell is a type of lymphocyte which develops in the thymus gland and plays a central role in the immune response. T cells can be distinguished from other lymphocytes by the presence of a T cell receptor on the cell surface. These immune cells originate as precursor cells, derived from bone marrow, and develop into several distinct types of T cells once they have migrated in to the thymus gland. T cell differentiation continues even after they have left the thymus. The lymphocyte of the present invention may be any T cell, for example a helper CD4⁺ T cell, a cytotoxic CD8⁺ T cell, a memory T cell, a regulatory CD4⁺ T cell, a natural killer T cell or a gamma delta T cell. In certain embodiments, the T cell according to the invention is a CD4⁺ or CD8⁺ T cell, optionally wherein the CD4⁺ or CD8⁺ T cell comprises a chimeric antigen receptor.

Natural killer cells, or NK cells, are a type of cytotoxic lymphocyte critical to the innate immune system. The role NK cells play is analogous to that of cytotoxic T cells in the vertebrate adaptive immune response. NK cells provide rapid responses to virus-infected cells, acting at around 3 days after infection, and respond to tumor formation. They were named “natural killers” because of the initial notion of the ability to lyse tumor cells without prior sensitization. Inhibitory receptors of NK cells engage mostly major histocompatibility class I (MHC class 1) molecules that are ubiquitously expressed on the surface of nucleated cells. Healthy cells expressing high levels of MHC class 1 sustain self-tolerance and are protected from NK cell killing. By contrast viral infection or malignant transformation triggers NK cell activation by removing inhibitory signals. Activating NK cell receptors recognized stress-induced ligands on virus-infected or malignant cells. Expression of these stimulatory ligands on target cells can overcome constitutive inhibition delivered by inhibitory receptors and thus activate NK cells.

Within the present invention, the lymphocyte may be any lymphocyte. Preferably, the lymphocyte of the invention is a T cell or NK cell. Thus, in one embodiment, the invention relates to the lymphocyte according to the invention, wherein the lymphocyte is a T cell or an NK cell. In another embodiment, the invention relates to the lymphocyte according to the invention, wherein the lymphocyte is a cytotoxic CD8⁺ T cell, a helper CD4⁺ T cell or an NK cell. In a further embodiment, the invention relates to the lymphocyte according to the invention, wherein the lymphocyte is a cytotoxic CD8⁺ T cell or an NK cell. In a further embodiment, the invention relates to the lymphocyte according to the invention, wherein the lymphocyte is a cytotoxic CD8⁺ T cell. In another embodiment, the invention relates to the lymphocyte according to the invention, wherein the lymphocyte is a helper CD4⁺ T cell

In one embodiment, the invention relates to the lymphocyte according to the invention, wherein the lymphocyte is a tumor infiltrating lymphocyte, a T cell comprising a modified TCR or a virus-specific T cell.

That is, the lymphocyte is a lymphocyte that is suitable for cell therapy applications, such as a TIL, a T cell comprising a modified TCR or a virus-specific T cell. Preferably, the TIL, the T cell comprising the modified TCR or the virus-specific T cell comprises a synthetic polynucleotide encoding at least one iron regulatory protein, preferably wherein the iron regulatory protein is IRP1 and/or IRP2, more preferably wherein the iron regulatory protein is IRP2, even more preferably wherein the iron regulatory protein is IRP2 as set forth in SEQ ID NO:2.

In certain embodiments, the lymphocyte of the invention may be a tumor-infiltrating lymphocyte (TIL). TILs are white blood cells that have left the bloodstream and migrated towards a tumor. They include T cells and B cells and are part of the larger category of ‘tumor-infiltrating immune cells’ which consist of both mononuclear and polymorphonuclear immune cells, (e.g., T cells, B cells, natural killer cells, macrophages, neutrophils, dendritic cells, mast cells, eosinophils, basophils, etc.) in variable proportions. Their abundance varies with tumor type and stage and in some cases relates to disease prognosis. TILs may be used in cell therapy, wherein TILs are isolated from a patient's tumor and expanded ex vivo. The expanded TILs may then be assayed for specific tumor recognition and the tumor specific TILs may then be re-infused into the patient, optionally after an additional expansion step.

Within the present invention, the synthetic polynucleotide encoding the at least one iron regulatory protein may be introduced ex vivo into TILs that have been obtained from a patient, in particular from the tumor of a patient. IRP overexpressing TILs may then be infused into a patient in the course of cancer therapy, preferably after one or more additional expansion and/or selection steps.

In certain embodiments of the invention, the lymphocyte of the invention may be a T cell comprising a modified T cell receptor (TCR). The term “T cell comprising a modified T cell receptor”, or “TCR-modified T cell”, refers to a T cell that has been genetically modified such that it expresses a specific TCR. TCR-modified T cells may be generated by obtaining a population of T cells from a subject and introducing a genetic element encoding a T cell receptor in said population of T cells. The TCR may be a naturally occurring TCR or an engineered TCR.

TCR-modified T cells may be used in cell therapy to increase a patient's immune response to a specific antigen, for example an antigen that has been verified to be produced by a tumor in said patient. Overexpressing an iron regulatory protein, preferably IPR1 and/or IPR2, more preferably IPR2, in a population of TCR-modified T cells may result in more robust proliferation of these T cells in vivo when infused into a patient and thus may elicit a stronger immune response against the antigen that is recognized by the TCR in said patient. The TCR-modified T cell of the invention may be any type of T cell. Preferably, the TCR-modified T cell of the invention is a CD4⁺ or a CD8⁺ T cell.

In certain embodiments of the invention, the lymphocyte according to the invention is a virus-specific T cell. A “virus-specific T cell” is a T cell, for example a CD4⁺ or CD8⁺ T cell, that has been stimulated with a viral antigen. When administered to a patient, a virus-specific T cell may be used to treat viral infections in the patient. Overexpressing an iron regulatory protein, preferably IRP1 and/or IRP2, more preferably IRP2, in a population of virus-specific T cells may result in more robust in vivo proliferation of these T cells when infused into a patient and thus may elicit a stronger immune response against the antigen that is recognized by the TCR in said patient.

In another embodiment, the invention relates to the lymphocyte according to the invention, wherein the at least one iron regulatory protein is constitutively expressed.

The at least one IRP that is encoded in the synthetic polynucleotide and comprised in the cell according the invention may be operably linked to any promoter or to any regulatory element known in the art. Accordingly, the at least one IRP may be constitutively expressed, i.e. expressed in most cell types at most times, or may be inducibly expressed, i.e. expressed only under certain physiological conditions and/or in response to a specific signal and/or inducer molecule. However, within the present invention, it is preferred that the at least one IRP is constitutively expressed. Alternatively, the at least one IRP may be inducibly expressed under conditions that are frequently encountered in cell therapy applications, for example, by signals, molecules and/or processes that are associated with activation of the lymphocyte.

The term “expression”, as used herein, refers to the production of a desired end-product molecule in a target cell. The end-product molecule may include, for example, an RNA molecule, a peptide, a protein or combinations thereof. Within the present invention, the end-product is preferably an iron regulatory protein.

A “constitutive” promoter is a nucleotide sequence which, when operably linked with a polynucleotide which encodes or specifies a gene product, causes the gene product to be produced in a cell under most or all physiological conditions of the cell.

One example of a suitable promoter is the immediate early cytomegalovirus (CMV) promoter sequence. This promoter sequence is a strong constitutive promoter sequence capable of driving high levels of expression of any polynucleotide sequence operatively linked thereto.

Another example of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Accordingly, in certain embodiments, the invention relates to the lymphocyte according to the invention, wherein the synthetic polynucleotide encoding the at least one iron regulatory protein is under control of a constitutive promoter.

A polynucleotide encoding a protein or polypeptide is “under control of a constitutive promoter”, if the constitutive promoter is responsible for initiating transcription of the polynucleotide encoding said protein or polypeptide. The skilled person is aware of methods for placing a polynucleotide encoding a protein or polypeptide under control of a promoter, for example by methods of molecular cloning.

The constitutive promoter may be any constitute promoter known in the art, preferably a constitutive promoter that initiates transcription in mammalian cells and, more preferably, in human cells. For example, the constitutive promoter may be any one of the constitutive promoters listed above.

In certain embodiments of the invention, the invention relates to the lymphocyte according to the invention, wherein the constitutive promoter is an EF-1α promoter.

Human elongation factor-1 alpha (EF-1 alpha) is a constitutive promoter of human origin that can be used to drive ectopic gene expression in various in vitro and in vivo contexts. Without being bound to theory, EF-1 alpha is often useful in conditions where other promoters (such as CMV) have diminished activity or have been silenced (as in embryonic stem cells).

An “inducible” promoter is a nucleotide sequence which, when operably linked with a polynucleotide which encodes or specifies a gene product, causes the gene product to be produced in a cell substantially only when an inducer which corresponds to the promoter is present in the cell. Examples of inducible promoters include, but are not limited to a metallothionine promoter, a glucocorticoid promoter, a progesterone promoter, and a tetracycline promoter.

In yet another embodiment, the invention relates to the lymphocyte according to the invention, wherein the at least one iron regulatory protein is IRP1 (SEQ ID NO: 1) and/or IRP2 (SEQ ID NO: 2-6).

That is, the at least one IRP that is encoded by the synthetic polynucleotide comprised in the lymphocyte according the invention may be any IRP known in the art. However, it is preferred that the IRP is human IRP1 and/or human IRP2. In humans, four different isoforms of IRP2 have been described, wherein two different sequences have been described for isoform 3 (SEQ ID NO: 2-6). Accordingly, in certain embodiments of the invention, the lymphocyte according to the invention may comprise a synthetic polynucleotide encoding a protein with the amino acid sequence of SEQ ID NO:1. In other embodiments of the invention, the lymphocyte according to the invention may comprise one or more synthetic polynucleotide(s) encoding a protein with the amino acid sequence of SEQ ID NO:2-6. In further embodiments of the invention, the lymphocyte according to the invention may comprise a synthetic polynucleotide encoding proteins with the amino acid sequences of SEQ ID NO:1 and one or more of SEQ ID NO:2-6. The lymphocyte according to the invention may also comprise two or more synthetic polynucleotides, wherein a first synthetic polynucleotide encodes a protein with an amino acid sequence of SEQ ID NO:1 and wherein a second or any further synthetic polynucleotide encodes a protein with an amino acid sequence of SEQ ID NO:2-6.

In one embodiment of the invention, the invention relates to the lymphocyte according to the invention, wherein the at least one iron regulatory protein is IRP1 (SEQ ID NO: 1). In another embodiment of the invention, the invention relates to the lymphocyte according to the invention, wherein the at least one iron regulatory protein is IRP2 (SEQ ID NO: 2). In yet another embodiment of the invention, the invention relates to the lymphocyte according to the invention, wherein the at least one iron regulatory protein is IRP2 (SEQ ID NO:3). In yet another embodiment of the invention, the invention relates to the lymphocyte according to the invention, wherein the at least one iron regulatory protein is IRP2 (SEQ ID NO:4). In yet another embodiment of the invention, the invention relates to the lymphocyte according to the invention, wherein the at least one iron regulatory protein is IRP2 (SEQ ID NO:5). In yet another embodiment of the invention, the invention relates to the lymphocyte according to the invention, wherein the at least one iron regulatory protein is IRP2 (SEQ ID NO:6). In another embodiment of the invention, the invention relates to the lymphocyte according to the invention, wherein the at least one iron regulatory protein is IRP1 (SEQ ID NO:1) and IRP2 (SEQ ID NO: 2). In a preferred embodiment of the invention, the invention relates to the lymphocyte according to the invention, wherein the at least one iron regulatory protein is IRP2 (SEQ ID NO: 2).

It has been demonstrated by the inventors that overexpressing IRP2 in lymphocytes results in more robust proliferation of these lymphocytes. It has further been shown by the inventors that silencing IRP1 in lymphocytes has similar effects as silencing IRP2, even though the effects are less significant than in the case of IRP2 (FIGS. 8C and D) However, it has to be noted that silencing of IRP1 was less efficient than silencing of IRP2 (FIG. 8A). Thus, it is plausible that overexpression of IRP1 may also result in increased proliferation of lymphocytes. Further, it is plausible that simultaneous overexpression of IRP1 and IRP2 may result in increased proliferation of lymphocytes.

In one embodiment, the invention relates to the lymphocyte according to the invention, wherein the lymphocyte further comprises a chimeric antigen receptor.

The term “chimeric antigen receptor” or “CAR” or “CARs”, as used herein, refers to engineered receptors, which graft an antigen specificity onto lymphocytes, for example T cells and NK cells. The CAR of the invention may be any CAR known in the art. Preferably, the CAR of the invention comprises at least one extracellular antigen binding domain, a transmembrane domain, one or more co-stimulatory signaling regions, and an intracellular signaling domain. In certain embodiments of the invention, the CAR may be a bispecific CAR, which is specific to two different antigens or epitopes. After the antigen binding domain binds specifically to a target antigen, the signaling domain activates intracellular signaling. For example, the signaling domain may redirect T cell specificity and reactivity towards a selected target in a non-MHC-restricted manner, exploiting the antigen-binding properties of antibodies. The non-MHC-restricted antigen recognition gives T cells expressing the CAR the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains. In case of NK cells, expression of a CAR may facilitate directing the NK cell to a target antigen. However, in contrast to CAR T cells, CAR NK cells may retain the expression of their activating and inhibitory receptors. Thus, different from CAR-T cells, CAR-NK cells may still exert their “natural” functions in case the antigen targeted by the CAR is downregulated.

Within the present invention, a lymphocyte is said to comprise a chimeric antigen receptor, if the lymphocyte comprises the coding sequences encoding the CAR and expresses these coding sequences such that the CAR is anchored to the membrane of the lymphocyte. The coding sequences encoding the components of the CAR may be located on one or more synthetic polynucleotides. In certain embodiments of the invention, the coding sequences encoding the components of the CAR and the one or more coding sequences encoding the IRPs may be located on a single synthetic polynucleotide. Alternatively, the coding sequences encoding the components of the CAR and the coding sequence(s) encoding the one or more IRPs may be located on two or more separate polynucleotides. For example, the polynucleotide encoding the CAR and the polynucleotide encoding the one or more IRPs may be introduced into the cell by two independent viral transduction events and may thus be integrated in different parts of the genome. The one or more synthetic polynucleotides comprising the CAR coding sequences, and optionally the IRP coding sequence(s), may be comprised in the lymphocyte in any form and may have been introduced into the lymphocyte by any method known in the art. For example, the synthetic polynucleotide encoding the CAR and/or the one of more iron regulatory proteins may be present inside the cell as part of a circular DNA vector, e.g. a plasmid, in form or as part of a linear DNA or in form or as part of an mRNA. However, it is preferred that the one or more synthetic polynucleotides comprising the CAR coding sequences, and optionally the IRP coding sequence(s), are integrated as DNA into the genome of the lymphocyte. The skilled person is aware of methods to introduce DNA into the genome of a lymphocyte. The synthetic polynucleotide encoding IRP1 and/or IRP2, and optionally the CAR, may be introduced into the genome by any method known in the art. In certain embodiments, the synthetic polynucleotide encoding IRP1 and/or IRP2, and optionally the CAR, may be introduced into the genome of the lymphocyte by viral transduction. However, other methods to introduce synthetic DNA into the genome of a lymphocyte, such as CRISPR/Cas9 are also encompassed herein.

In another embodiment, the invention relates to the lymphocyte according to the invention, wherein the chimeric antigen receptor comprises an antigen binding domain, a transmembrane domain, a co-stimulatory signaling region and a signaling domain.

The lymphocyte according to the invention may comprise a chimeric antigen receptor (CAR) comprising an extracellular and intracellular domain. The extracellular domain may comprise one or more target-specific binding elements otherwise referred to as an antigen binding moiety. The intracellular domain or otherwise the cytoplasmic domain may comprise one or more co-stimulatory signaling regions and a signaling domain. The co-stimulatory signaling region refers to a portion of the CAR comprising the intracellular domain of a co-stimulatory molecule. Co-stimulatory molecules are cell surface molecules other than antigen receptors or their ligands that are required for an efficient response of lymphocytes to an antigen.

Between the extracellular domain and the transmembrane domain of the CAR, or between the cytoplasmic domain and the transmembrane domain of the CAR, there may be incorporated a spacer domain. As used herein, the term “spacer domain” generally means any oligo- or polypeptide that functions to link the transmembrane domain to either the extracellular domain or the cytoplasmic domain in the polypeptide chain. A spacer domain may comprise up to 300 amino acids, preferably 10 to 100 amino acids and most preferably 25 to 50 amino acids.

The CAR of the invention may comprise one or more target-specific binding element(s) otherwise referred to as an antigen binding moiety. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen moiety domain in the CAR of the invention include those associated with viral, bacterial and parasitic infections, autoimmune disease and cancer cells.

Depending on the desired antigen to be targeted, the CAR of the invention may be engineered to include the appropriate antigen binding moiety that is specific to the desired antigen target. For example, if CD19 is the desired antigen that is to be targeted, an antibody for CD19 may be used as the antigen binding moiety for incorporation into the CAR of the invention.

With respect to the transmembrane domain, the CAR may be designed to comprise a transmembrane domain that is fused to the extracellular domain of the CAR. In certain embodiments, a transmembrane domain that is naturally associated with the extracellular or cytoplasmic domain of the CAR may be used. In some instances, the transmembrane domain may be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD8, CD3 epsilon, CD45, CD4, CD5, CD8, CD9, CD16, CD22, CD33, CD37, CD64, CD80, CD86, CD134, CD137, CD154. Alternatively, the transmembrane domain may be synthetic, in which case it will comprise predominantly hydrophobic residues such as leucine and valine. Preferably, a triplet of phenylalanine, tryptophan and valine will be found at each end of a synthetic transmembrane domain.

Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

The cytoplasmic domain or otherwise the intracellular signaling domain of the CAR of the invention is responsible for activation of at least one of the normal effector functions of the lymphocyte in which the CAR has been placed in. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal. Preferred examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequences: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs (ITAMs).

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the invention comprises a cytoplasmic signaling sequence derived from CD3 zeta.

The cytoplasmic domain of the CAR may be designed to comprise the CD3-zeta signaling domain by itself or combined with any other desired cytoplasmic domain(s) useful in the context of the CAR of the invention. For example, the cytoplasmic domain of the CAR may comprise a CD3 zeta chain portion and a co-stimulatory signaling region. The co-stimulatory signaling region refers to a portion of the CAR comprising the intracellular domain of a co-stimulatory molecule. A co-stimulatory molecule is a cell surface molecule other than an antigen receptor or their ligands that is required for an efficient response of lymphocytes to an antigen. Examples of such molecules include CD27, CD28, 4-1 BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

The cytoplasmic signaling sequences within the cytoplasmic signaling portion of the CAR of the invention may be linked to each other in a random or specified order. Optionally, a short oligo- or polypeptide linker, preferably between 2 and 10 amino acids in length may form the linkage. A glycine-serine doublet provides a particularly suitable linker.

In certain embodiments, the cytoplasmic domain may be designed to comprise the signaling domain of CD3-zeta and the signaling domain of CD28 and/or 4-1BB.

In yet another embodiment, the invention relates to the lymphocyte according to the invention, wherein the antigen binding domain is an antibody or an antigen-binding fragment thereof, in particular wherein the antigen-binding fragment is a Fab or an scFv.

That is, the antigen binding domain of the CAR may be any domain known in the art that is capable of specifically binding a specific antigen. However, it is preferred that the antigen binding domain of the CAR is an antibody or an antigen-binding fragment of an antibody.

The term “antibody,” as used herein, refers to an immunoglobulin molecule which specifically binds to an antigen. Antibodies may be intact immunoglobulins derived from natural sources or from recombinant sources or may be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston, et al., 1988, Proc. Natl. Acad. Sci. USA; 85:5879-5883; Bird, et al., 1988, Science; 242:423-426).

The term “immunoglobulin” or “Ig”, as used herein, is defined as a class of proteins, which function as antibodies. Antibodies expressed by B cells are sometimes referred to as the BCR (B cell receptor) or antigen receptor. The Five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects, it is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

The term “Fab” as used herein is intended to refer to a region of an antibody composed of one constant and one variable domain of each of the heavy and the light chains (monovalent antigen-binding fragment), but wherein the heavy chain is truncated such that it lacks the CH2 and CH3 domain and may also lack some or all of the hinge region. A Fab fragment may be produced by digestion of a whole antibody with the enzyme papain. Fab may refer to this region in isolation, or this region in the context of a full length antibody, immunoglobulin construct or Fab fusion protein.

By “scFv” it is meant an antibody fragment comprising the VH and VL domains of an antibody, wherein these domains are present in a single polypeptide chain. See, for example, U.S. Pat. Nos. 4,946,778, 5,260,203, 5,455,030, and 5,856,456. Generally, the Fv polypeptide further comprises a polypeptide linker between the VH and VL domains that enables the scFv to form the desired structure for antigen-binding. For a review of scFv see Pluckthun (1994) The Pharmacology of Monoclonal Antibodies vol 113 ed. Rosenburg and Moore (Springer-Verlag, New York) pp 269-315. The VH and VL domain complex of Fv fragments may also be stabilized by a disulfide bond (U.S. Pat. No. 5,747,654).

An “antibody heavy chain,” as used herein, refers to the larger of the two types of polypeptide chains present in all antibody molecules in their naturally occurring conformations, An “antibody light chain,” as used herein, refers to the smaller of the two types of polypeptide chains present in all antibody molecules in their naturally occurring conformations, and X light chains refer to the two major antibody light chain isotypes.

By the term “synthetic antibody”, as used herein, is meant an antibody which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The skilled person is aware of methods for generating CARs with varying antigen-binding domains, transmembrane domains, co-stimulatory signaling regions and/or signaling domains. Further, the skilled person is aware of methods to introduce such CARs into a lymphocyte, such as a T cell or an NK cell.

In one embodiment, the invention relates to the lymphocyte according to the invention, wherein the antigen binding domain specifically binds a tumor antigen.

That is, the antigen binding domain of the CAR may bind to any antigen known in the art. However, it is preferred that the antigen binding domain of the CAR specifically binds to a tumor antigen. The term “antigen” or “Ag”, as used herein, is defined as a molecule that provokes an immune response. This immune response may involve either antibody production, or the activation of specific immunologically-competent cells, or both. The skilled artisan will understand that any macromolecule, including virtually all proteins or peptides, may serve as an antigen. Furthermore, antigens may be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen may be generated, synthesized or may be derived from a biological sample. Such a biological sample may include, but is not limited to a tissue sample, a tumor sample, a cell or a biological fluid.

Tumor antigens are proteins that are produced by tumor cells that elicit an immune response, particularly T cell-mediated immune responses. The selection of the antigen binding moiety of the CAR will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulm, RAGE-1, MN-CA IX, human telomerase reverse transcriptase, RU1, RU2 (AS), intestinal carboxylesterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

The tumor antigen may comprise one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD 19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-1), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erb-B3, c-met, nm23_H1, PSA, TAG-72, CA 19-9, CA 72-4, CAM17.1, NuMa, K-ras, beta-Catenin, CDK4, Mum-1, p15, p16, 43-9F, 5T4 (791Tgp72), alpha-fetoprotein, beta-HCG, BCA225, BTAA, CA 125, CA 15-3\CA 27.29\BCAA, CA 195, CA 242, CA-50, CAM43, CD68\I, CO-029, FGF-5, G250, Ga733\EpCAM, HTgp-175, M344, MA-50, MG7-Ag, MOV 18, NB/70K, NY-CO-1, RCAS-1, SDCCAG16, TA-90\Mac-2 binding protein, cyclophilin C-associated protein, TAAL6, TAG72, TLP, and TPS.

The antigen binding moiety portion of the CAR may further target an antigen that includes but is not limited to CD19, CD20, CD22, CD30, CD123, CD171, CS-1, ROR1, Mesothelin, CD33, IL3Ra, c-Met, PSMA, Glycolipid F77, EGFRvIII, GD-2, CD7, NY-ESO-1 TCR, MAGE-A3 TCR, CLL-1, GD3, BCMA, Tn Ag, PSMA, FLT3, FAP, TAG72, CD38, CD44v6, CEA, EPCAM, B7H3, KIT, IL-13Ra2, IL-IRa, PSCA, PRSS21, VEGFR2, LewisY, CD24, PDGFR-beta, SSEA-4, Folate receptor alpha, ErbB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GMI, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLACI, Globo H, NY-BR-1, UPK2, HAVCRI, ADRB3, PANX3, GPR20, LY6K, ORS1E2, TARP, WT1, LAGE-1a, legumain, HPV E6, E7, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1 and the like.

In certain embodiments, the lymphocyte of the invention comprising a CAR may be used in the treatment of blood cancers, in particular in the treatment of acute lymphoblastic leukemia and/or diffuse large B-cell lymphoma. In these embodiments, the antigen binding moiety portion of the CAR may specifically target CD19.

In certain embodiments, the lymphocyte of the invention comprising a CAR may be used in the treatment of blood cancers, in particular in the treatment of refractory Hodgkin's lymphoma. In these embodiments, the antigen binding moiety portion of the CAR may specifically target CD30.

In certain embodiments, the lymphocyte of the invention comprising a CAR may be used in the treatment of blood cancers, in particular in the treatment of acute myeloid leukemia. In these embodiments, the antigen binding moiety portion of the CAR may specifically target CD33, CD123 or FLT3.

In certain embodiments, the lymphocyte of the invention comprising a CAR may be used in the treatment of blood cancers, in particular in the treatment of multiple myeloma. In these embodiments, the antigen binding moiety portion of the CAR may specifically target BCMA.

In certain embodiments, the CAR comprised in the lymphocyte of the invention may bind to two antigens. In certain embodiments, the bi-specific CAR may bind to CD19 and CD22 or to CD19 and CD20.

Generally, CARs have the advantage to not depend on presentation of a tumor antigen by an MHC molecule on the surface of the target cell. Instead, CARs can bind, in theory, to any molecule that is accessible for the CAR on the surface of a tumor cell, provided that the antigen-binding domain of the CAR specifically binds the antigen. Thus, the tumor antigen is preferably an antigen that is present on the surface of a tumor or malignant cell. More preferably, the tumor antigen is an antigen that is more abundant on the surface of tumor or malignant cells than it is on the surface of a healthy or non-tumor cell. Even more preferably, the tumor antigen is an antigen that is present on the surface of tumor cells or malignant cells but is absent on the surface of healthy or non-tumor cells.

By the term “specifically binds,” as used herein with respect to an antigen binding domain or an antibody, is meant an antigen binding domain or an antibody which recognizes a specific antigen but does not substantially recognize or bind other molecules in a sample. For example, an antigen binding domain or antibody that specifically binds to an antigen from one species may also bind to that antigen from one or more other species. But, such cross-species reactivity does not itself alter the classification of an antigen binding domain or antibody as specific. In another example, an antigen binding domain or antibody that specifically binds to an antigen may also bind to different allelic forms of the antigen. However, such cross reactivity does not itself alter the classification of an antibody as specific. In some instances, the terms “specific binding” or “specifically binding,” may be used in reference to the interaction of an antigen binding domain, an antibody, a protein, or a peptide with a second chemical species, to mean that the interaction is dependent upon the presence of a particular structure (e.g., an antigenic determinant or epitope) on the chemical species; for example, an antigen binding domain or an antibody recognizes and binds to a specific protein structure rather than to proteins generally. If an antigen binding domain or an antibody is specific for epitope “A”, the presence of a molecule containing epitope A (or free, unlabeled A), in a reaction containing labeled “A” and the antigen binding domain or antibody, will reduce the amount of labeled A bound to the antigen binding domain or antibody.

In another embodiment, the invention relates to the lymphocyte according to the invention, wherein the tumor antigen is present on the surface of cells of the target cell population or tissue.

The lymphocyte according to the invention comprising a CAR may bind to a tumor antigen that is present on the cell surface of a target cell. The target cell may be part of a cell population or a tissue. In general, a tumor antigen is said to be “present on the cell surface” if the tumor antigen is exposed by the target cell such that the tumor antigen is accessible to the antigen binding domain of the CAR.

The tumor antigen may be any protein that is produced by a tumor cell or, more preferably, any part of a protein that is produced by a tumor cell and expressed on the cell surface of said tumor cell. It is preferred that the tumor antigen is part of the extracellular domain of a membrane-anchored protein that is accessible to the antigen binding domain of the CAR.

However, the present invention also encompasses tumor antigens that are presented on the surface of the target cell by another molecule, in particular an MHC molecule. In this case, the tumor antigen is preferably a peptide that is derived from a protein. The tumor antigen may, for example, be derived from a protein that is produced by a tumor cell. Alternatively, the tumor antigen may be derived from an extracellular protein that has previously been taken up by a tumor cell, for example by endocytosis. In both cases, the proteins may be processed by the target cell to peptides, which then may be presented on the surface of the target cell, for example by an MHC molecule.

In one embodiment, the invention relates to the lymphocyte according to the invention, wherein the antigen binding domain specifically binds a viral antigen.

That is, the lymphocyte according to the invention may be used in the treatment of viral infections in a subject. The CAR that is comprised in the lymphocyte of the invention may comprise an antigen binding domain that specifically binds a viral antigen. The viral antigen may be any component of a virus particle that is accessible to the CAR, for example an antigen that forms part of the surface and/or the protein coat of the virus. Preferably, the viral antigen that is recognized by the CAR is an antigen derived from human immunodeficiency virus (HIV), adenoviruses, polyomaviruses, influenza virus or human herpesvirus, in particular wherein the human herpesvirus is cytomegalovirus (CMV), Epstein-Barr virus (EBV), herpes simplex virus (HSV), Varizella-Zoster virus (VZV) or human herpesvirus 8 (HHV8).

In one embodiment, the invention relates to the lymphocyte according to the invention, wherein the CAR is encoded by a polynucleotide and wherein the polynucleotide encoding the CAR is transcriptionally linked to the synthetic polynucleotide encoding IRP1 and/or IRP2.

Within the present invention, it is preferred that the CAR is encoded by a polynucleotide that is integrated into the genome of the lymphocyte. More preferably, the polynucleotide encoding the CAR and the polynucleotide encoding IRP1 and/or IRP2 are integrated into the same locus of the genome of the lymphocyte. More preferably, the polynucleotide encoding the CAR and the polynucleotide encoding IRP1 and/or IRP2 are integrated into the same locus of the genome of the lymphocyte such that the two or more polynucleotides are transcriptionally linked. Two or more polynucleotides are said to be transcriptionally linked, if transcription of the coding sequences comprised in the two or more polynucleotides is driven from a single promoter, such that a single transcript encoding two or more polypeptides is obtained. Preferably, the promoter is located upstream (5′) of the coding sequences or polynucleotides that are transcriptionally linked. That is, the coding sequence encoding the CAR and the coding sequence(s) encoding IRP1 and/or IRP2 may be transcribed from a single promoter.

To enable synthesis of functional proteins, the coding sequence encoding the CAR and the coding sequence(s) encoding IRP1 and/or IRP2 may be separated by an internal ribosome entry site (IRES) or may be connected by a polynucleotide encoding a self-cleaving peptide.

When the coding sequence encoding the CAR and the coding sequence(s) encoding IRP1 and/or IRP2 are separated by an IRES, each coding sequence comprised in the transcript is independently translated. If, however, the coding sequence encoding the CAR and the coding sequence(s) encoding IRP1 and/or IRP2 are connected by a self-cleaving peptide, the entire transcript is translated into a polyprotein, which is then cleaved into single proteins during or subsequent to translation by means of auto cleavage.

In one embodiment, the invention relates to the lymphocyte according to the invention, wherein the polynucleotide encoding the CAR and the synthetic polynucleotide encoding IRP1 and/or IRP2 are linked by a polynucleotide encoding a self-cleaving peptide.

The term “self-cleaving peptide” as used herein refers to a peptide sequence that is associated with a cleavage activity that occurs between two amino acid residues within the peptide sequence itself. For example, in the 2A/2B peptide or in the 2A/2B-like peptides, cleavage occurs between the glycine residue on the 2A peptide and a proline residue on the 2B peptide. This occurs through a ‘ribosomal skip mechanism’ during translation wherein normal peptide bond formation between the 2A glycine residue and the 2B proline residue of the 2A/2B peptide is impaired, without affecting the translation of the rest of the 2B peptide. Such ribosomal skip mechanisms are well known in the art and are known to be used by several viruses for the expression of several proteins encoded by a single messenger RNA.

Thus, in one embodiment, the invention relates to the lymphocyte according to the invention, wherein the self-cleaving peptide is a 2A self-cleaving peptide.

In a preferred embodiment, the invention relates to the lymphocyte according to the invention, wherein the self-cleaving peptide is T2A. T2A is a self-cleaving peptide comprising the peptide sequence EGRGSLLTCGDVEENPGP (SEQ ID NO:7).

When two coding sequences are linked by a polynucleotide encoding a self-cleaving peptide, it is to be understood that the coding sequence encoding a first polypeptide, the coding sequence encoding the self-cleaving peptide and the coding sequence encoding a second polypeptide are encoded in the same reading frame.

Within the present invention, it is preferred that the polynucleotide encoding the CAR and the polynucleotide(s) encoding IRP1 and/or IRP2 are encoded on the same synthetic polynucleotide. In certain embodiments, the synthetic polynucleotide encoding the CAR, IRP1 and/or IRP2 have been integrated into the genome of the lymphocyte by viral transduction. In certain embodiments, the polynucleotide encoding the CAR and the polynucleotide encoding IRP1 comprised in the synthetic polynucleotide are separated by an IRES. In another embodiments, the polynucleotide encoding the CAR and the polynucleotide encoding IRP2 comprised in the synthetic polynucleotide are separated by an IRES. In other embodiments, the polynucleotide encoding the CAR and the polynucleotide encoding IRP1 comprised in the synthetic polynucleotide are connected by a polynucleotide encoding a self-cleaving peptide, in particular a 2A self-cleaving peptide, in particular T2A. In other embodiments, the polynucleotide encoding the CAR and the polynucleotide encoding IRP2 comprised in the synthetic polynucleotide are connected by a polynucleotide encoding a self-cleaving peptide, in particular a 2A self-cleaving peptide, in particular T2A.

In certain embodiments, the synthetic polynucleotide encoding the CAR, IRP1 and/or IRP2 is under control of a constitutive promoter. In certain embodiments, the promoter is part of the synthetic polynucleotide. In certain embodiments, the constitutive promoter is an EF-1α promoter. However, it is to be understood that the skilled person is aware of a wide range of promoters that may be used instead of the promoter EF-1α. Further, it is to be understood that Example 10 merely represents a proof of concept and that more efficient in vivo proliferation of lymphocytes may be achieved by optimizing the expression of the CAR and/or IRP1/2 in the lymphocytes.

In certain embodiments, the synthetic polynucleotide has the structure: 5′-CAR-self-cleaving peptide-IRP1-3′. In other embodiments, the synthetic polynucleotide has the structure: 5′-CAR-self-cleaving peptide-IRP2-3′. In other embodiments, the synthetic polynucleotide has the structure: 5′-constitutive promoter-CAR-self-cleaving peptide-IRP1-3′. In other embodiments, the synthetic polynucleotide has the structure: 5′-constitutive promoter-CAR-self-cleaving peptide-IRP2-3′. In other embodiments, the synthetic polynucleotide has the structure: 5′-constitutive promoter-CAR-T2A-IRP1-3′. In other embodiments, the synthetic polynucleotide has the structure: 5′-constitutive promoter-CAR-T2A-IRP2-3′.

In certain embodiments, the synthetic polynucleotide has the structure: 5′-IRP1-self-cleaving peptide-CAR-3′. In other embodiments, the synthetic polynucleotide has the structure: 5′-IRP2-self-cleaving peptide-CAR-3′. In other embodiments, the synthetic polynucleotide has the structure: 5′-constitutive promoter-IRP1-self-cleaving peptide-CAR-3′. In other embodiments, the synthetic polynucleotide has the structure: 5′-constitutive promoter-IRP2-self-cleaving peptide-CAR-3′. In other embodiments, the synthetic polynucleotide has the structure: 5′-constitutive promoter-IRP1-T2A-CAR-3′. In other embodiments, the synthetic polynucleotide has the structure: 5′-constitutive promoter-IRP2-T2A-CAR-3′. In other embodiments, the synthetic polynucleotide has the structure: 5′-constitutive promoter-IRP1-P2A-CAR-3′. In other embodiments, the synthetic polynucleotide has the structure: 5′-constitutive promoter-IRP2-P2A-CAR-3′. In other embodiments, the synthetic polynucleotide has the structure: 5′-constitutive promoter-IRP1-E2A-CAR-3′. In other embodiments, the synthetic polynucleotide has the structure: 5′-constitutive promoter-IRP2-E2A-CAR-3′. In other embodiments, the synthetic polynucleotide has the structure: 5′-constitutive promoter-IRP1-F2A-CAR-3′. In other embodiments, the synthetic polynucleotide has the structure: 5′-constitutive promoter-IRP2-F2A-CAR-3′.

However, it is to be understood that the invention also encompasses lymphocytes wherein a first polynucleotide encoding IRP1 and/or IRP2 and a second polynucleotide encoding the CAR are integrated into different locations of the genome of the lymphocyte and are expressed independently. Preferably, the polynucleotide encoding IRP1 and/or IRP2 and the polynucleotide encoding the CAR are integrated into the genome of the lymphocyte by two independent viral transduction events. The two independent viral transduction events may have occurred simultaneously or may have occurred in a step-wise manner.

In another embodiment, the invention relates to a viral vector comprising at least one polynucleotide encoding IRP1 (SEQ ID NO: 1) and/or IRP2 (SEQ ID NO: 2-6).

That is, the invention further relates to viral vectors that may be used for integrating an iron regulatory protein into a cell, preferably a lymphocyte. The viral vector may be any viral vector that is suitable for the integration of polynucleotides into a cell, preferably a lymphocyte. Thus, in certain embodiments, the invention relates to the viral vector according to the invention, wherein the viral vector is derived from a lentivirus, an adeno-associated virus (AAV), an adenovirus, a herpes simplex virus, a retrovirus, an alphavirus, a flavivirus, a rhabdovirus, a measles virus, a Newcastle disease virus or a poxvirus. In a preferred embodiment, the invention relates to the viral vector according to the invention, wherein the viral vector is derived from a lentivirus or an adeno-associated virus (AAV). In a more preferred embodiment, the invention relates to the viral vector according to the invention, wherein the viral vector is derived from a lentivirus.

The viral vector may comprise one or more transgenes. The term “transgene” as used herein refers to particular nucleic acid sequences encoding a polypeptide or a portion of a polypeptide to be expressed in a cell into which the nucleic acid sequence is inserted. The term transgene is meant to include (1) a nucleic acid sequence that is not naturally found in the cell (i.e., a heterologous nucleic acid sequence, such as a nucleic acid encoding a CAR); (2) a nucleic acid sequence that is a mutant form of a nucleic acid sequence naturally found in the cell into which it has been introduced; (3) a nucleic acid sequence that serves to add additional copies of the same (i.e., homologous) or a similar nucleic acid sequence naturally occurring in the cell into which it has been introduced (such as IRP1 and/or IRP2; or (4) a silent naturally occurring or homologous nucleic acid sequence whose expression is induced in the cell into which it has been introduced. By mutant form is meant a nucleic acid sequence that contains one or more nucleotides that are different from the wild-type or naturally occurring sequence, i.e., the mutant nucleic acid sequence contains one or more nucleotide substitutions, deletions, and/or insertions. In some cases, the transgene may also include a sequence encoding a leader peptide or signal sequence such that the transgene product will be secreted from the cell.

The synthetic polynucleotide encoding IRP1 and/or IRP2 and, optionally, a promoter and/or a CAR that is comprised in the lymphocyte according to the invention may preferably be integrated into the lymphocyte by means of viral transduction. Thus, it is to be understood that the synthetic polynucleotide that has been disclosed for the lymphocyte according to the invention may also be comprised in the viral vector according to the invention.

In certain embodiments, the viral vector comprises a single transgene. For example, in certain embodiments, the viral vector comprises a polynucleotide encoding IRP1 (SEQ ID NO:1). In other embodiments, the viral vector comprises a polynucleotide encoding IRP2 (SEQ ID NO:2). In other embodiments, the viral vector comprises a polynucleotide encoding IRP2 (SEQ ID NO:3). In other embodiments, the viral vector comprises a polynucleotide encoding IRP2 (SEQ ID NO:4). In other embodiments, the viral vector comprises a polynucleotide encoding IRP2 (SEQ ID NO:5). In other embodiments, the viral vector comprises a polynucleotide encoding IRP2 (SEQ ID NO:6). Preferably, the viral vector comprises a polynucleotide encoding IRP2 (SEQ ID NO:2).

In certain embodiments, the viral vector may comprise more than one transgene. For example, the viral vector may comprise two or more polynucleotides encoding IRP1 (SEQ ID NO:1) and one or more isotypes of IRP2 (SEQ ID NOs:2-6). In another embodiment, the viral vector may comprise two or more polynucleotides encoding two tor more isotypes of IRP2 (SEQ ID NOs:2-6).

Further, the viral vector may comprise one or more polynucleotides encoding IRP1 and/or IRP2 and an additional polynucleotide encoding a CAR. Thus, in one embodiment, the invention relates to the viral vector according to the invention, wherein the viral vector comprises a further polynucleotide encoding a CAR. Accordingly, the viral vector may be used to integrate a polynucleotide encoding the CAR and at least one polynucleotide encoding IRP1 and/or IRP2 into a cell, preferably a lymphocyte, simultaneously.

In certain embodiments, the invention relates to the viral vector according to the invention, wherein the polynucleotide encoding the CAR is transcriptionally linked to the polynucleotide(s) encoding IRP1 and/or IRP2. The polynucleotide encoding the CAR and the polynucleotide(s) encoding IRP1 and/or IRP2 may be transcriptionally linked as described above. That is, the polynucleotide encoding the CAR and the polynucleotide(s) encoding IRP1 and/or IRP2 may be under control of a common promoter.

The polynucleotide encoding the CAR and the one or more polynucleotides encoding IRP1 and/or IRP2 may be separated by one or more IRES or may be connected by one or more polynucleotides encoding self-cleaving peptides as described herein.

In certain embodiments, the invention relates to the viral vector according to the invention, wherein the polynucleotide encoding the CAR and the polynucleotide(s) encoding IRP1 and/or IRP2 are linked by a polynucleotide encoding a self-cleaving peptide.

In certain embodiments, the invention relates to the viral vector according to the invention, wherein the self-cleaving peptide is a 2A self-cleaving peptide.

In certain embodiments, the invention relates to the viral vector according to the invention, wherein the self-cleaving peptide is T2A.

In another embodiment, the viral vector may further comprise a promoter that controls the expression of the one or more polynucleotides encoding the CAR and IRP1 and/or IRP2. Thus, in another embodiment, the invention relates to the viral vector according to the invention, wherein the at least one polynucleotide encoding IRP1 and/or IRP2 and, optionally the CAR, is under control of a promoter. The promoter may be a constitutive promoter or an inducible promoter, for example one of the constitutive or inducible promoters specified elsewhere herein. Preferably, the promoter is a constitutive promoter, such as the promoter EF-1α. Thus, in a certain embodiment, the invention relates to the viral vector according to the invention, wherein the constitutive promoter is an EF-1α promoter.

In certain embodiments, the viral vector may comprise a polynucleotide having the structure: 5′-CAR-self-cleaving peptide-IRP1-3′. In other embodiments, the viral vector may comprise a polynucleotide having the structure: 5′-CAR-self-cleaving peptide-IRP2-3′. In other embodiments, the viral vector may comprise a polynucleotide having the structure: 5′-constitutive promoter-CAR-self-cleaving peptide-IRP1-3′. In other embodiments, the viral vector may comprise a polynucleotide having the structure: 5′-constitutive promoter-CAR-self-cleaving peptide-IRP2-3′. In other embodiments, the viral vector may comprise a polynucleotide having the structure: 5′-constitutive promoter-CAR-T2A-IRP1-3′. In other embodiments, the viral vector may comprise a polynucleotide having the structure: 5′-constitutive promoter-CAR-T2A-IRP2-3′. In other embodiments, the viral vector may comprise a polynucleotide having the structure: 5′-constitutive promoter-CAR-P2A-IRP1-3′.

In other embodiments, the viral vector may comprise a polynucleotide having the structure: 5′-constitutive promoter-CAR-P2A-IRP2-3′. In other embodiments, the viral vector may comprise a polynucleotide having the structure: 5′-constitutive promoter-CAR-E2A-IRP1-3′. In other embodiments, the viral vector may comprise a polynucleotide having the structure: 5′-constitutive promoter-CAR-E2A-IRP2-3′. In other embodiments, the viral vector may comprise a polynucleotide having the structure: 5′-constitutive promoter-CAR-F2A-IRP1-3′. In other embodiments, the viral vector may comprise a polynucleotide having the structure: 5′-constitutive promoter-CAR-F2A-IRP2-3′.

In certain embodiments, the viral vector may comprise a polynucleotide having the structure: 5′-IRP1-self-cleaving peptide-CAR-3′. In other embodiments, the viral vector may comprise a polynucleotide having the structure: 5′-IRP2-self-cleaving peptide-CAR-3′. In other embodiments, the viral vector may comprise a polynucleotide having the structure: 5′-constitutive promoter-IRP1-self-cleaving peptide-CAR-3′. In other embodiments, the viral vector may comprise a polynucleotide having the structure: 5′-constitutive promoter-IRP2-self-cleaving peptide-CAR-3′. In other embodiments, the viral vector may comprise a polynucleotide having the structure: 5′-constitutive promoter-IRP1-T2A-CAR-3′. In other embodiments, the viral vector may comprise a polynucleotide having the structure: 5′-constitutive promoter-IRP2-T2A-CAR-3′. In other embodiments, the viral vector may comprise a polynucleotide having the structure: 5′-constitutive promoter-IRP1-P2A-CAR-3′. In other embodiments, the viral vector may comprise a polynucleotide having the structure: 5′-constitutive promoter-IRP2-P2A-CAR-3′. In other embodiments, the viral vector may comprise a polynucleotide having the structure: 5′-constitutive promoter-IRP1-E2A-CAR-3′. In other embodiments, the viral vector may comprise a polynucleotide having the structure: 5′-constitutive promoter-IRP2-E2A-CAR-3′. In other embodiments, the viral vector may comprise a polynucleotide having the structure: 5′-constitutive promoter-IRP1-F2A-CAR-3′. In other embodiments, the viral vector may comprise a polynucleotide having the structure: 5′-constitutive promoter-IRP2-F2A-CAR-3′.

The skilled person is aware of molecular biology methods to introduce transgenes and/or regulatory elements such as promoters into a viral vector.

In one embodiment, the invention relates to a pharmaceutical composition comprising the lymphocyte according to the invention and a pharmaceutically acceptable carrier.

The lymphocytes of the present invention may be administered either alone, or as a pharmaceutical composition comprising the lymphocyte of the present invention. Briefly, pharmaceutical compositions of the present invention may comprise a lymphocyte or a population of lymphocytes as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. The pharmaceutical composition according to the invention may be administered in combination with diluents and/or with other components such as IL-2 or other cytokines or cell populations. Compositions of the present invention are preferably formulated for intravenous administration.

In another embodiment, the invention relates to a pharmaceutical composition comprising the viral vector according to the invention and a pharmaceutically acceptable carrier.

In certain embodiments direct treatment of a subject by direct introduction of the vector is contemplated. The viral vector compositions may be formulated for delivery by any available route including, but not limited to parenteral (e.g., intravenous), intradermal, subcutaneous, oral (e.g., inhalation), transdermal (topical), transmucosal, rectal, and vaginal. Commonly used routes of delivery include inhalation, parenteral, and transmucosal.

In certain embodiments, the pharmaceutical composition according to the invention may comprise the viral vector according to the invention comprising a polynucleotide encoding at least one iron regulatory protein and a second viral vector comprising a polynucleotide encoding a CAR. That is, the polynucleotide(s) encoding the one or more iron regulatory proteins and the polynucleotide encoding the CAR may be located on two separate viral vectors but may be comprised in the same pharmaceutical composition.

In various embodiments pharmaceutical compositions can include a viral vector in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

In some embodiments, active agents, i.e., a viral vector described herein and/or other agents to be administered together the vector, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such compositions will be apparent to those skilled in the art. Suitable materials can also be obtained commercially from Alza Corporation and Nova Pharmaceuticals, Inc. Liposomes can also be used as pharmaceutically acceptable carriers. These can be prepared according to methods known to those skilled in the art, for example, as described in U.S. Pat. No. 4,522,811. In some embodiments the composition is targeted to particular cell types or to cells that are infected by a virus. For example, compositions can be targeted using monoclonal antibodies to cell surface markers, e.g., endogenous markers or viral antigens expressed on the surface of infected cells.

It is advantageous to formulate compositions in dosage unit form for ease of administration and uniformity of dosage. Dosage unit form as used herein refers to physically discrete units suited as unitary dosages for the subject to be treated; each unit comprising a predetermined quantity of a viral vector calculated to produce the desired therapeutic effect in association with a pharmaceutical carrier.

A unit dose need not be administered as a single injection but may comprise continuous infusion over a set period of time. Unit dose of the viral vector described herein may conveniently be described in terms of transducing units (T.U.) of viral vector, as defined by titering the vector on a cell line such as HeLa or 293. In certain embodiments unit doses can range from 10³, 10⁴, 10⁵, 10⁶, 10⁷, 10⁸, 10⁹, 10¹⁰, 10¹¹, 10¹², 10¹³ T.U. and higher.

Pharmaceutical compositions can be administered at various intervals and over different periods of time as required, e.g., one time per week for between about 1 to about 10 weeks; between about 2 to about 8 weeks; between about 3 to about 7 weeks; about 4 weeks; about 5 weeks; about 6 weeks, etc. It may be necessary to administer the therapeutic composition on an indefinite basis. The skilled artisan will appreciate that certain factors can influence the dosage and timing required to effectively treat a subject, including but not limited to the severity of the disease or disorder, previous treatments, the general health and/or age of the subject, and other diseases present. Treatment of a subject with a viral vector can include a single treatment or, in many cases, can include a series of treatments.

Exemplary doses for administration of viral vectors and methods for determining suitable doses are known in the art. It is furthermore understood that appropriate doses of a viral vector may depend upon the particular recipient and the mode of administration. The appropriate dose level for any particular subject may depend upon a variety of factors including the age, body weight, general health, gender, and diet of the subject, the time of administration, the route of administration, the rate: of excretion, other administered therapeutic agents, and the like.

In certain embodiments viral vectors can be delivered to a subject by, for example, intravenous injection, local administration, or by stereotactic injection (see, e.g., Chen et al. (1994) Proc. Natl. Acad. Sci. USA, 91: 3054). In certain embodiments vectors may be delivered orally or by inhalation and may be encapsulated or otherwise manipulated to protect them from degradation, enhance uptake into tissues or cells, etc. Pharmaceutical preparations can include a viral vector in an acceptable diluent, or can comprise a slow release matrix in which a viral vector is imbedded. Alternatively or additionally, where a vector can be produced intact from recombinant cells, as is the case for retroviral or lentiviral vectors, a pharmaceutical preparation can include one or more cells which produce vectors. Pharmaceutical compositions comprising a viral vector described herein can be included in a container, pack, or dispenser, optionally together with instructions for administration.

The foregoing compositions, methods and uses are intended to be illustrative and not limiting. By using the teachings provided herein other variations on the compositions, methods and uses will be readily available to one of skill in the art.

In another embodiment, the invention relates to the lymphocyte according to the invention, the viral vector according to the invention or the pharmaceutical composition according to the invention for use in therapy.

That is, the lymphocyte according to the invention, the viral vector according to the invention or a pharmaceutical composition comprising the lymphocyte and/or the viral vector according to the invention may be used in therapy.

In certain embodiments, the invention relates to the lymphocyte according to the invention, the viral vector according to the invention or the pharmaceutical composition according to the invention for use in treating cancer.

The invention provides the use of a CAR as defined in the invention to redirect the specificity of a lymphocyte, for example a T cell or an NK cell, to a tumor antigen. Disclosed herein is a type of cellular therapy, wherein lymphocytes are genetically modified to express at least one IRP and a CAR, wherein the resulting cells are infused to a recipient in need thereof. The infused cell is able to kill tumor cells in the recipient. Unlike antibody therapies, the lymphocytes according to the invention are able to replicate in vivo resulting in long-term persistence that can lead to sustained tumor control.

Due to the overexpression of the at least one IRP, the lymphocytes described herein can undergo robust in vivo expansion and can persist for an extended amount of time. Without wishing to be bound by any particular theory, the anti-tumor immunity response elicited by the lymphocytes of the invention may be an active or a passive immune response. In addition, the CAR mediated immune response may be part of an adoptive immunotherapy approach in which CAR-modified lymphocytes induce an immune response specific to the antigen binding moiety in the CAR.

While lymphocytes that express at least one IRP and a CAR are preferred for the treatment of cancer, this may also be envisioned by lymphocytes expressing only the at least one IRP, but no CAR. In this case, the synthetic polynucleotide encoding the at least one IRP may be introduced into a lymphocyte ex vivo and the genetically engineered lymphocyte may then be administered to a subject having cancer. Optionally, the lymphocyte may be stimulated with a tumor antigen ex vivo to increase the specificity for a certain type of cancer or tumor. In certain embodiments, the genetically engineered lymphocyte expressing at least one IRP may be injected directly into a tumor.

However, it is to be understood that the invention also encompasses the use of lymphocytes overexpressing IRP1 and/or IRP2 but no CAR in the treatment of cancer.

For example, IRP1 and/or IRP2 may be overexpressed in TILs or TCR-modified T cells which are subsequently used in cell therapy. It has been demonstrated by the inventors that overexpression of IRPs in lymphocytes results in more robust proliferation of lymphocytes. Thus, it is plausible that overexpressing IRPs in TILs or TCR-modified T cells results in more effective cell therapies.

Further, IRP1 and/or IRP2 may be overexpressed in NK cells which are subsequently used in cell therapy. In certain embodiments, the NK cell is an allogenic NK cell.

To “treat” a disease, as the term is used herein, means to reduce the frequency or severity of at least one sign or symptom of a disease or disorder experienced by a subject.

A “disease” is a state of health of an animal (including humans) wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “cancer”, as used herein is, defines as disease characterized by the rapid and uncontrolled growth of aberrant cells. Cancer cells can spread locally or through the bloodstream and lymphatic system to other parts of the body.

Cancers that may be treated with the lymphocytes according to the invention include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the lymphocytes of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

In one embodiment, the invention relates to the lymphocyte, the viral vector or the pharmaceutical composition for use according to the invention, wherein the cancer is a hematologic cancer or a solid tumor. In a particular embodiment the hematologic cancer is acute lymphoblastic leukemia, diffuse large B-cell lymphoma, Hodgkin's lymphoma, acute myeloid leukemia or multiple myeloma and the solid tumor is colon cancer, breast cancer, pancreatic cancer, ovarian cancer, hepatocellular carcinoma, lung cancer, neuroblastoma, glioblastoma or sarcoma.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

The lymphocyte of the invention may be designed to target CD19 and may be used to treat cancers and disorders including but are not limited to pre-B ALL (pediatric indication), adult ALL, mantle cell lymphoma, diffuse large B-cell lymphoma, salvage post allogenic bone marrow transplantation, and the like.

The CAR-modified lymphocytes described herein may also serve as a type of vaccine for ex vivo immunization and/or in vivo therapy in a subject. Preferably, the subject is a human.

With respect to ex vivo immunization, at least one of the following occurs in vitro prior to administering the lymphocyte into a subject: i) expansion of the cells, ii) introducing at least one synthetic nucleotide encoding at least one IRP and/or a CAR into the cells, and/or iii) cryopreservation of the cells.

Ex vivo procedures are well known in the art. Briefly, lymphocytes are isolated from a subject (preferably a human) and genetically modified (i.e., transduced or transfected in vitro) with at least one vector expressing at least one IRP and/or a CAR disclosed herein. The CAR-modified cell expressing the at least one IRP may be administered to a recipient to provide a therapeutic benefit. The recipient may be a human and the modified lymphocyte may be autologous with respect to the recipient. Alternatively, the lymphocytes may be allogeneic, syngeneic or xenogeneic with respect to the recipient.

The procedure for ex vivo expansion of hematopoietic stem and progenitor cells described in U.S. Pat. No. 5,199,942 may be applied to the cells of the present invention. Other suitable methods are known in the art, therefore the present invention is not limited to any particular method of ex vivo expansion of the cells. Briefly, ex vivo culture and expansion of lymphocytes comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand may be used for culturing and expansion of the cells.

In addition to using a cell-based vaccine in terms of ex vivo immunization, the present disclosure also provides compositions and methods for in vivo immunization to elicit an immune response directed against an antigen in a patient.

Generally, the lymphocytes activated and expanded as described herein may be utilized in the treatment and prevention of diseases that arise in individuals who are immunocompromised. In particular, the CAR-modified lymphocytes described herein may be used in the treatment of chronic lymphocytic leukemia (CCL). In certain embodiments, the lymphocytes described herein may be used in the treatment of patients at risk for developing CCL. Thus, the present disclosure provides for the treatment or prevention of CCL comprising administering to a subject in need thereof, a therapeutically effective amount of a lymphocyte of the invention.

Alternatively, the lymphocyte for use in the treatment of cancer may be an NK cell, a TIL or a TCR-modified lymphocyte. NK cells, TILs or TCR-modified T cell may modified with the methods of the invention such that they overexpress IRP1 and/or IRP2, which may result in more efficient proliferation of the NK cell, the TIL or the TCR-modified T-cell in vivo.

NK cells used in cancer therapy may be allogeneic NK cells, since allogenic NK cells have graft-versus-leukemia/tumor (GvL/GvT) effects without causing graft-versus-host disease (GvHD), thus cause less immunopathology.

TILs for use in cancer therapy may be obtained as described in WO 2018/182817 and may be further modified by introducing at least one polynucleotide encoding at least one IRP.

In one embodiment, the invention relates to the lymphocyte according to the invention, the viral vector according to the invention or the pharmaceutical composition according to the invention for use in preventing and/or treating viral infections.

That is, the lymphocyte according to the invention may also be used in the prevention and treatment of viral infections. It is known that virus-specific T cells can be used to prevent or treat viral infections, for example, but not exclusively, in subjects that received hematopoietic stem cell transplantations. Virus-specific T cells may be generated by stimulating and expanding T cells with a viral antigen, for example an antigen-presenting cell displaying a viral antigenic peptide, a whole virus particle, viral lysates, whole viral proteins or viral vectors. Alternatively, a virus-specific T cell may be generated by expressing a natural or engineered T cell receptor that is known to bind a specific viral antigen. The resulting virus-specific T cells may then be administered to a subject that is suffering from a viral infection or is at risk of acquiring a viral infection.

Expressing at least one IRP in a virus-specific T cell, thereby creating a pseudo iron-deficient state, may result in virus-specific T cells that proliferate more robustly after the virus-specific T cells have been administered to the subject. In consequence, virus-specific T cells that have been genetically engineered to express at least one IRP may be more efficient at preventing or treating viral infections compared to non-genetically engineered virus specific T cells. The synthetic polynucleotide encoding the at least one IRP may be introduced into the T cell before, during or after the T cell has been stimulated with the viral antigen.

Prevention and treatment of viral infections as described above does not necessarily require the presence of a CAR. However, using a lymphocyte according to the invention that further comprises a CAR may, at least in some instances, improve the recognition of a virus by a lymphocyte. In this case, the CAR may preferably comprise an antigen binding domain that specifically binds a viral antigen.

In one embodiment, the invention relates to the lymphocyte, the viral vector or pharmaceutical composition for use according to the invention, wherein the viral infection is caused by a human immunodeficiency virus (HIV), adenoviruses, polyomaviruses, influenza virus or human herpesvirus, in particular wherein the human herpesvirus is cytomegalovirus (CMV), Epstein-Barr virus (EBV), herpes simplex virus (HSV), Varizella-Zoster virus (VZV) or human herpesvirus 8 (HHV8).

Human cytomegalovirus is a pervasive s-herpes virus with prevalence rates of 50-100% in the general population. While it may manifest as mild self-limiting disease in the immunocompetent host, CMV can cause severe life-threatening disease in the immunocompromised host. Because CMV persists in the latent form after acute infection, CMV-specific CD4+ and CD8+ T cells are necessary to maintain viral quiescence. In post-HSCT patients, in the absence of donor immunity and in other immunodeficient states, CMV may reactivate in the form of retinitis, pneumonitis, hepatitis, or enterocolitis. The adoptive transfer of CMV-specific T cells is a logical strategy for treating and preventing CMV reactivation in such individuals, and numerous clinical trials confirm the overall excellent efficacy of virus specific T cells. CMV-specific VSTs generated from naive T cells in umbilical cord blood (UCB) have also proved effective. These VSTs show specificity for atypical epitopes while maintaining functionality.

EBV is a ubiquitous, highly immunogenic γ-herpesvirus that can cause unique complications following transplant. Over 90% of the general population have been infected and retain lifelong seropositivity. Manifestations of primary EBV infection vary widely from asymptomatic infection to a debilitating viral illness. Thereafter in most cases, EBV remains latent lifelong in a B cell and mucosal epithelial reservoir under continuous T cell immune surveillance. In these healthy individuals, up to 2% of circulating T cells are EBV specific. In the period of immune deficiency after HSCT, EBV reactivation may cause viremia and life-threatening posttransplant lymphoproliferative disease (PTLD). While the monoclonal antibody rituximab successfully treats severe EBV disease in many patients by eliminating B cells in which the EBV virus resides, it results in long-term reduction in antibody production and is not always successful at controlling PTLD.

Adenovirus infection can range from mild upper respiratory tract infections to a spectrum of life-threatening pneumonia, gastrointestinal, hepatic, renal, and neurologic complications. Following infection, latency is maintained in the lymphoid tissues, but the virus can reactivate during periods of prolonged absence of T cell immunity. Adenovirus causes potentially lethal viral complication in post-HSCT recipients. Antiviral drugs such as ribavirin are largely ineffective. However, adenovirus-specific T cells generated from healthy donors have proven effective at treating even advanced disease. For this reason, adenovirus antigens are often incorporated in the generation of multivirus-specific T cell products.

The BK and JC polyomaviruses, normally latent in healthy tissues of most adult individuals, reactivate after HSCT and in immunodeficient individuals. BK virus may manifest as nephropathy and life-threatening hemorrhagic cystitis (HC). Rarely, the closely associated JC virus causes fatal brain damage from progressive multifocal leukoencephalopathy. Polyoma-specific VST are being developed to combat these viruses. A single case report describes the successful use of BK VSTs, after which the patient had complete resolution of HC without bystander organ toxicity, GVHD, or graft rejection. It is clear that the platforms developed for ex vivo selected and expanded VSTs are readily adaptable to many other viruses that complicate immune deficient states, and future developments include developing VST to target an array of viruses including VZV, HHV, and even HIV or influenza.

In one embodiment, the invention relates to a method for treating a subject having cancer or for preventing and/or treating a viral infection in a subject, the method comprising administering to the subject a therapeutically effective amount of the lymphocyte according to the invention, the viral vector according to the invention or the pharmaceutical composition according to the invention.

Lymphocytes or pharmaceutical compositions described herein may be administered in a manner appropriate to the disease to be treated (or prevented). The quantity and frequency of administration will be determined by such factors as the condition of the patient, and the type and severity of the patient's disease, although appropriate dosages may be determined by clinical trials.

An “effective amount” as used herein, means an amount which provides a therapeutic or prophylactic benefit. The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc, of the subject to be treated.

When “an immunologically effective amount”, “an anti-tumor effective amount”, “a tumor-inhibiting effective amount”, or “therapeutic amount” is indicated, the precise amount of the lymphocytes or the compositions of the present invention to be administered may be determined by a physician with consideration of individual differences in age, weight, tumor size, extent of infection or metastasis, type of viral infection, severity of viral infection and/or condition of the patient (subject). It may generally be stated that a pharmaceutical composition comprising the lymphocytes described herein may be administered at a dosage of 10⁴ to 10⁹ cells/kg body weight, preferably 10⁵ to 10⁶ cells/kg body weight, including all integer values within those ranges. Lymphocyte compositions may also be administered multiple times at these dosages. The lymphocytes may be administered by using infusion techniques that are commonly known in immunotherapy (Rosenberg, et al., 1988, New Eng. J. of Med; 319:1676.). The optimal dosage and treatment regime for a particular patient may readily be determined by one skilled in the art of medicine by monitoring the patient for signs of disease and adjusting the treatment accordingly.

It may be desired to administer activated lymphocytes to a subject and then subsequently redraw blood (or have an apheresis performed), activate lymphocytes therefrom according to the present invention, and reinfuse the patient with these activated and expanded lymphocytes. This process can be carried out multiple times every few weeks. Lymphocytes may be activated from blood draws of from 10 mL to 400 mL, for example, lymphocytes may be activated from blood draws of 20 mL, 30 mL, 40 mL, 50 mL, 60 mL, 70 mL, 80 mL, 90 mL, or 100 mL. Not to be bound by theory, using this multiple blood draw/multiple reinfusion protocol may serve to select out certain populations of lymphocytes.

The administration of the lymphocytes or compositions may be carried out in any convenient manner, including by aerosol inhalation, injection, ingestion, transfusion, implantation or transplantation. The lymphocytes or compositions described herein may be administered to a subject subcutaneously, intradermally, intratumorally, intranodally, intramedullary, intramuscularly, by intravenous (i.v.) injection, or intraperitoneally. For example, the lymphocytes or compositions described herein may be administered to a patient by intradermal or subcutaneous injection. In another example, the lymphocytes or compositions described herein may preferably be administered by i.v. injection. The lymphocytes or compositions may be injected directly into a tumor, lymph node, or site of infection.

In certain instances, lymphocytes activated and expanded using the methods described herein, or other methods known in the art where lymphocytes are expanded to therapeutic levels, are administered to a patient in conjunction with (e.g., before, simultaneously or following) any number of relevant treatment modalities, including but not limited to treatment with agents such as antiviral therapy, cidofovir and interleukin-2, ribavirin, rituximab, Cytarabine (also known as ARA-C) or natalizumab treatment for MS patients or efalizumab treatment for psoriasis patients or other treatments for PML patients. Also disclosed herein, the lymphocytes of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu, et al., 1991, Cell; 66:807-815; Henderson et al., 1991, Immun; 73:316-321; Bierer et al., 1993, Curr. Opin. Immun; 5:763-773). Also disclosed herein, the lymphocytes or compositions of the present invention may be administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. Also described herein, the lymphocytes or compositions of the present invention may be administered following B-cell ablative therapy such as agents that react with CD20, e.g., Rituxan. For example, subjects may undergo standard treatment with high dose chemotherapy followed by peripheral blood stem cell transplantation. In certain instances, following the transplant, subjects may receive an infusion of the expanded immune cells of the present invention, or expanded cells are administered before or following surgery.

The dosage of the above treatments to be administered to a subject will vary with the precise nature of the condition being treated and the recipient of the treatment. The scaling of dosages for human administration may be performed according to art-accepted practices.

In one embodiment, the invention relates to the method according to the invention, wherein the cancer is a hematologic cancer or a solid tumor, in particular wherein the hematologic cancer is acute lymphoblastic leukemia, diffuse large B-cell lymphoma, Hodgkin's lymphoma, acute myeloid leukemia and multiple myeloma or wherein the solid tumor is colon cancer, breast cancer, pancreatic cancer, ovarian cancer, hepatocellular carcinoma, lung cancer, neuroblastoma, gliosblastoma and sarcoma.

In one embodiment, the invention relates to the method according to the invention, wherein the viral infection is caused by a human immunodeficiency virus (HIV), adenoviruses, polyomaviruses, influenza virus or human herpesvirus, in particular wherein the human herpesvirus is cytomegalovirus (CMV), Epstein-Barr virus (EBV), herpes simplex virus (HSV), Varizella-Zoster virus (VZV) or human herpesvirus 8 (HHV8).

In one embodiment, the invention relates to a method for producing the lymphocyte according to the invention, the method comprising the steps of: a) providing a lymphocyte obtained from a subject; b) introducing a synthetic polynucleotide encoding at least one iron regulatory protein into the lymphocyte of step (a), wherein the iron regulatory protein is IRP1 (SEQ ID NO:1) and/or IRP2 (SEQ ID NOs: 2-6) and c) expressing the at least one iron regulatory protein encoded by the synthetic polynucleotide that has been introduced into the lymphocyte in step (b). It is to be understood that the lymphocyte may be any lymphocyte disclosed herein.

Optionally, the invention further relates to the method according to the invention, wherein a second synthetic polynucleotide encoding a chimeric antigen receptor (CAR) is introduced into the lymphocyte in step (b).

That is, the method according to the invention may be used for producing a lymphocyte that overexpresses IRP1 and/or IRP2. Further, the method according to the invention may be used to produce lymphocytes comprising two synthetic polynucleotides, a first synthetic polynucleotide encoding IRP1 and/or IRP2 and a second synthetic polynucleotide encoding a CAR. In the latter case, it is to be understood that the first synthetic polynucleotide encoding IRP1 and/or IRP2 and the second synthetic polynucleotide encoding the CAR may be fused to each other as described herein. Alternatively, the first synthetic polynucleotide encoding IRP1 and/or IRP2 and the second synthetic polynucleotide encoding the CAR may be unrelated. That is, the first synthetic polynucleotide encoding IRP1 and/or IRP2 and the second synthetic polypeptide encoding the CAR may be introduced into the lymphocyte independently. Preferably, the synthetic polynucleotides are introduced into the lymphocyte by viral transduction and incorporated into the genome of the lymphocyte. Accordingly, the first synthetic polynucleotide encoding IRP1 and/or IRP2 and the second synthetic polypeptide encoding the CAR may be comprised in different viral vectors. The first viral vector comprising the synthetic polynucleotide encoding IRP1 and/or IRP2 and the second viral vector comprising the synthetic polynucleotide encoding the CAR may be introduced into the lymphocyte in a single transduction experiment. Alternatively, the lymphocyte may be transduced with the first viral vector comprising the synthetic polynucleotide encoding IRP1 and/or IRP2 and the second viral vector comprising the synthetic polynucleotide encoding the CAR in a step-wise manner. For example, the lymphocyte according to the invention may first be transduced with a viral vector comprising a synthetic polynucleotide encoding a CAR to produce, without limitation, a CAR T cell or a CAR NK cell, and may in a second step be transduced with a viral vector comprising a synthetic polynucleotide encoding IRP1 and/or IRP2. Alternatively, the lymphocyte may first be transduced with a viral vector comprising a synthetic polynucleotide encoding IRP1 and/or IRP2 and may in a second step be transduced with a viral vector comprising a synthetic polynucleotide encoding a CAR.

Prior to genetic modification of the lymphocytes of the invention, a source of lymphocytes may be obtained from a subject. Lymphocytes may be obtained from a number of sources, including peripheral blood, mononuclear cells, bone marrow, lymph node tissue, blood, thymus tissue, tissue from a site of infection, ascites, pleural effusion, spleen tissue, and tumors. Within the present invention, any type of lymphocyte available in the art may be used. In general, the skilled person is aware of methods to isolate a certain type of lymphocyte from a suitable source.

Certain types of lymphocytes, specifically peripheral blood mononuclear cells (PBMCs) may be obtained from a unit of blood collected from a subject using any number of techniques known to the skilled artisan, such as Ficoll™ separation. Further, cells from the circulating blood of an individual may be obtained by apheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps. For example, the cells may be washed with phosphate buffered saline (PBS).

Alternatively, the wash solution may lack calcium and may lack magnesium or may lack many if not all divalent cations. As those of ordinary skill in the art would readily appreciate, a washing step may be accomplished by methods known to those in the art, such as by using a semi-automated “flowthrough” centrifuge (for example, the Cobe 29 cell processor, the Baxter CytoMate, or the Haemonetics Cell Saver 5) according to the manufacturer's instructions. After washing, the cells may be resuspended in a variety of biocompatible buffers, such as, for example, Ca+-free, Mg+-free PBS, PlasmaLyte A, or other saline solution with or without buffer. Alternatively, the undesirable components of the apheresis sample may be removed and the cells may be directly resuspended in culture media. Alternatively, certain types of lymphocytes may be isolated by lysing red blood cells and depleting the monocytes, for example, by centrifugation through a PERCOLL™ gradient or by counterflow centrifugal elutriation.

A specific subpopulation of lymphocytes, such as CD3+, CD28+, CD4+, CD8+, CD45RA+, and CD45RO+ T cells, CD16+ and CD56+NK cells or CD3+, CD56+ and CD161+ NKT cells, may be further isolated by positive or negative selection techniques. It is known in the art, which surface antigens are present on the respective types of lymphocytes. Thus, the skilled person is capable of selecting conditions for positive or negative selection that allow for the enrichment or isolation of specific types of lymphocytes. In addition, the skilled person is aware of commercial kits for the enrichment and/or isolation of certain types of lymphocytes.

In certain embodiments, T cells may be isolated by incubation with anti-CD3/anti-CD28-conjugated beads, such as DYNABEADS® M-450 CD3/CD28 T, for a time period sufficient for positive selection of the desired T cell. The time period may range from 30 minutes to 36 hours or longer and may include all integer values in-between. In certain embodiments, the time period is at least 0.5, 1, 2, 3, 4, 5, or 6 hours. Alternatively, the time period is 10 to 24 hours. For isolation of T cells from patients with leukemia, use of longer incubation times, such as 24 hours, can increase cell yield. Longer incubation times may be used to isolate T cells in any situation where there are fewer T cells as compared to other cell types, such in isolating tumor infiltrating lymphocytes (TIL) from tumor tissue or from immune-compromised individuals. Further, use of longer incubation times may increase the efficiency of capture of CD8+ T cells. Thus, by simply shortening or lengthening the time T cells are allowed to bind to the CD3/CD28 beads and/or by increasing or decreasing the ratio of beads to T cells, subpopulations of T cells may be preferentially selected for or against at culture initiation or at other time points during the process, Additionally, by increasing or decreasing the ratio of anti-CD3 and/or anti-CD28 antibodies on the beads or other surface, subpopulations of T cells may be preferentially selected for or against at culture initiation or at other desired time points. The skilled artisan would recognize that multiple rounds of selection may also be used in the context of this invention. In certain embodiments, it may be desirable to perform the selection procedure and use the “unselected” cells in the activation and expansion process. “Unselected” cells may also be subjected to further rounds of selection.

Enrichment of a lymphocyte population by negative selection may be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+CD25+, CD62L, GITR+, and FoxP3+. Alternatively, in certain embodiments, T regulatory cells may be depleted by anti-CD25 conjugated beads or other similar method of selection.

In certain embodiments, NK cells from healthy donors or patients may be enriched from PBMCs or directly from blood by incubation with magnetic beads using the human NK cell negative selection isolation kit (Miltenyi Biotec or STEMCELL Technologies) according to the manufacturer's instructions. For example, when using isolation kits from Miltenyi Biotec, unwanted cells (i.e. T cells, B cells, macrophages and monocytes) may be removed with a cocktail of biotin-conjugated monoclonal anti-human antibodies against antigens not expressed by NK cells in a PBMC concentration of 2.5 billion cells/mL. Unwanted cells labeled with biotin-conjugated antibodies may then be magnetically labeled using NK cell MicroBead Cocktail and removed using MACS columns. For example, when using isolation kits from STEMCELL Technologies, unwanted cells (i.e. T cells, B cells, macrophages and monocytes) may be removed with tetrameric anti-human antibody complexes against antigens not expressed by NK cells in a PBMC concentration of 50 million cells/mL. Unwanted cells labeled with tetrameric antibody complexes may then be magnetically labeled with dextran-coated magnetic particles and removed using a magnet.

For isolation of a desired population of lymphocytes by positive or negative selection, the concentration of cells and surface (e.g., particles such as beads) may be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/mL may be used. In one embodiment, a concentration of 1 billion cells/mL may be used. In a further embodiment, greater than 100 million cells/mL may be used. In a further embodiment, a concentration of cells of 0, 15, 20, 25, 30, 35, 40, 45, or 50 million cells/mL may be used. In yet another embodiment, a concentration of cells from 75, 80, 85, 90, 95, or 100 million cells/mL may be used. In further embodiments, concentrations of 125 or 150 million cells/mL may be used. Using high concentrations may result in increased cell yield, cell activation, and cell expansion. Further, use of high cell concentrations may allow more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells may allow more efficient selection of CD8+ T cells that normally have weaker CD28 expression.

In a related embodiment, it may be desirable to use lower concentrations of cells. By significantly diluting the mixture of cells and surface (e.g., particles such as beads), interactions between the particles and cells may be minimized. This may select for cells that express high amounts of desired antigens to be bound to the particles. For example, CD4+ T cells may express higher levels of CD28 and are more efficiently captured than CD8+ T cells in dilute concentrations.

The lymphocytes may be incubated on a rotator for varying lengths of time at varying speeds at either 2-10° C. or at room temperature. Cells for stimulation may also be frozen after a washing step. Wishing not to be bound by theory, the freeze and subsequent thaw step may provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 1.25% Plasmalyte-A, 3.25% Dextrose 5%, 0.45% NaCl, 10% Dextran 40 and 5% Dextrose, 20% Human Serum Albumin, and 7.5% DMSO or other suitable cell freezing media containing, for example, Hespan and PlasmaLyte-A. The cells then may be frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

Cryopreserved cells may be thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded may be collected at any time point necessary, and desired cells, such as lymphocytes, isolated and frozen for later use in cell therapy for any number of diseases or conditions that would benefit from cell therapy, such as those described herein. A blood sample or an apheresis may be taken from a generally healthy subject. In certain embodiments, a blood sample or an apheresis may be taken from a generally healthy subject who is at risk of developing a disease, but who has not yet developed a disease, and the cells of interest may be isolated and frozen for later use. In certain embodiments, the cells may be expanded, frozen, and used at a later time. In certain embodiments, samples may be collected from a patient shortly after diagnosis of a particular disease as described herein but prior to any treatments. Further, the cells may be isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (99-101). In certain embodiments, the cells may be isolated from a patient and frozen for later use in conjunction with (e.g., before, simultaneously or following) bone marrow or stem cell transplantation, T cell ablative therapy using either chemotherapy agents such as fludarabine, external-beam radiation therapy (XRT), cyclophosphamide, or antibodies against OKT3 or CAMPATH. In certain embodiments, the cells may be isolated prior to and can be frozen for later use for treatment following B-cell ablative therapy such as agents that react with CD20, e.g., Rituxan.

In certain embodiments of the present invention, lymphocytes may be obtained from a patient directly following treatment. In this regard, it has been observed that following certain cancer treatments, in particular treatments with drugs that damage the immune system, shortly after treatment during the period when patients would normally be recovering from the treatment, the quality of lymphocytes obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including lymphocytes, dendritic cells, or other cells of the hematopoietic lineage, during this recovery phase. Further, in certain embodiments, mobilization (for example, mobilization with GM-CSF) and conditioning regimens may be used to create a condition in a subject wherein repopulation, recirculation, regeneration, and/or expansion of particular cell types is favored, especially during a defined window of time following therapy. Illustrative cell types include T cells, NK cells, B cells, dendritic cells, and other cells of the immune system.

The present invention encompasses one or more synthetic polynucleotides comprising polynucleotide sequences encoding one or more IRPs and, optionally, a CAR. The synthetic polynucleotides encoding the desired molecules may be obtained using recombinant methods known in the art, such as, for example by screening libraries from cells expressing the genes, by deriving the genes from a vector known to include the same, or by isolating directly from cells and tissues containing the same, using standard techniques. Alternatively, the genes of interest may be produced synthetically, rather than cloned.

The present invention also provides vectors in which a synthetic polynucleotide of the present invention may be inserted. Vectors derived from retroviruses such as lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

In brief summary, the expression of natural or synthetic polynucleotides encoding IRPs and, optionally, CARs, is typically achieved by operably linking a polynucleotide encoding the IRP or, optionally, the CAR polypeptide or portions thereof to a promoter, and incorporating the construct into an expression vector. The vectors may be suitable for replication and/or integration in eukaryotes. Typical cloning vectors contain transcription and translation terminators, initiation sequences, and promoters useful for regulation of the expression of the desired polynucleotides.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g. lentiviruses, retroviruses, adenoviruses, and adeno-associated viruses) that incorporate the recombinant polynucleotide.

The expression constructs of the present invention may also be used for nucleic acid immunization and gene therapy, using standard gene delivery protocols. Methods for gene delivery are known in the art.

The synthetic polynucleotide encoding the IRP and, optionally, the CAR may be cloned into a number of types of vectors. For example, the polynucleotide may be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al, (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene may be inserted into a vector and packaged in retroviral particles using techniques known in the art. The recombinant virus may then be isolated and delivered to cells of the subject either in vivo or ex vivo. A number of retroviral systems are known in the art. Within the present invention, it is preferred that adenovirus vectors or lentivirus vectors are used.

Additional promoter elements, e.g., enhancers, may be used to regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function may be preserved when elements are inverted or moved relative to one another. In the thymidine kinase (tk) promoter, the spacing between promoter elements may be increased to 50 bp apart before activity begins to decline.

Depending on the promoter, it appears that individual elements may function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters, inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of an IRP or, optionally, a CAR polypeptide or portions thereof, the expression vector to be introduced into a cell may also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene may be assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (Ui-Tei et al., 2000, FEBS Letters; 479: 79-82). Suitable expression systems are well known and may be prepared using known techniques or obtained commercially.

Methods of introducing and expressing genes into a cell are known in the art. In the context of an expression vector, the vector may be readily introduced into a host cell, e.g., mammalian, bacterial, yeast, or insect cell by any method in the art. For example, the expression vector may be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous polynucleotides are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle may be a liposome. The use of lipid formulations is contemplated for the introduction of the polynucleotides into a host cell (in vitro, ex vivo or in vivo). In another embodiment, the polynucleotide may be associated with a lipid. The polynucleotide associated with a lipid may be encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use may be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol may be stored at about −20° C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes may have multiple lipid layers separated by aqueous medium. They may form spontaneously when phospholipids are suspended in an excess of aqueous solution. The lipid components may undergo self-rearrangement before the formation of closed structures and entrap water and dissolved solutes between the lipid bilayers (Ghosh et al., 1991, Glycobiology; 5; 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Regardless of the method used to introduce exogenous synthetic polynucleotides into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR: “biochemical” assays, such as detecting the presence or absence of a particular peptide, e.g., by immunological means (ELISAs, Western blots, flow cytometry) or by assays described herein to identify agents falling within the scope of the invention.

In another embodiment, the invention relates to the method according to the invention, wherein the synthetic polynucleotide encoding the chimeric antigen receptor (CAR) is combined with the synthetic polynucleotide encoding the at least one iron regulatory protein, in particular wherein the at least one iron regulatory protein is IRP1 and/or TRP2.

The chimeric antigen receptor and the at least one IRP may be encoded on separate synthetic polynucleotides that are not contiguous or directly connected to each other. In this case, the synthetic polynucleotide encoding the at least one IRP and the synthetic polynucleotide encoding the CAR may be introduced into the cell separately using the same or different methods. Alternatively, the gene(s) encoding the at least one IRP and the genes encoding the CAR may be combined in a single synthetic polynucleotide. Two synthetic polynucleotides are said to be combined, if the combined synthetic polynucleotide comprises all genes that are encoded in the two separate synthetic polypeptides.

For example, if it is planned to integrate the genes encoding the at least one IRP and the CAR into the lymphocyte by viral transduction, the gene(s) encoding the at least one IRP and the genes encoding the CAR may be comprised in separate viral vectors or may be combined in a single viral vector.

In yet another embodiment, the invention relates to the method according to the invention, wherein the lymphocyte is activated before or after the one or more synthetic polynucleotide is introduced into the lymphocyte.

Whether prior to or after genetic modification of the lymphocytes to express at least one IRP or, optionally, a desirable CAR, lymphocytes may be activated and expanded before they are administered to a subject. The skilled person is aware that specific conditions are required to activate different types of lymphocytes.

The skilled person is aware of methods to activate NK cells. For example, the NK cells described herein may be activated by culturing the cells in appropriate medium (e.g. Minimal Essential Media or RPMI Media 1640 or, X-vivo 15, (Lonza), CellGro media (Cellgenix), IMDM (Gibco)) that may contain factors necessary for proliferation and viability, including serum (e.g., fetal bovine, human or horse serum) supplemented with IL-15 and/or IL-12 and/or IL-18. Activation of NK cells can also be accomplished by the supplementation of the media with IL-2. Activation of NK cells may be improved by adding a feeder cell line to the culture. Appropriate feeder cell lines for the activation of NK cells are cancer cell lines, genetically modified K562 cells, or EBV-transformed lymphoblastoid cell lines or autologous peripheral blood mononuclear cells (irradiated).

Generally, the T cells described herein may be activated by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule may be used. For example, a population of T cells may be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody may be used. Anti-CD28 antibodies 9.3, B-T3, XR-CD28 (Diaclone, Besangon, France) may be used, as can other methods commonly known in the art (Berg et al., 1998, Transplant Proc; 30(8):3975-3977; Haanen et al., 1999, J. Exp. Med; 190:13191328; Garland et al., 1999, J. Immunol Meth. 227:53-63).

The primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. For example, the agent providing the co-stimulatory signal may be bound to a surface and the agent providing the primary activation signal may be in solution or coupled to a surface, or both agents may be in solution. Alternatively, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells.

The two agents are immobilized on beads, either on the same bead, i.e., “cis,” or to separate beads, i.e., “trans.” By way of example, the agent providing the primary activation signal may be an anti-CD3 antibody or an antigen-binding fragment thereof and the agent providing the co-stimulatory signal may be an anti-CD28 antibody or antigen-binding fragment thereof; and both agents may be co-immobilized to the same bead in equivalent molecular amounts. For example, a 1:1 ratio of each antibody bound to the beads for CD4+ T cell expansion and T cell growth may be used. In certain instances, a ratio of anti CD3:CD28 antibodies bound to the beads may be used such that an increase in T cell expansion is observed as compared to the expansion observed using a ratio of 1:1. An increase of from about 1 to about 3 fold may be observed as compared to the expansion observed using a ratio of 1:1. The ratio of CD3:CD28 antibody bound to the beads may range from 100:1 to 1:100 and all integer values there between. In one instance, more anti-CD28 antibody may be bound to the particles than anti-CD3 antibody, i.e., the ratio of CD3:CD28 may be less than one. In certain instances, the ratio of anti CD28 antibody to anti CD3 antibody bound to the beads may be greater than 2:1. For example, a 1:100 CD3:CD28 ratio of antibody bound to beads may be used, a 1:75 CD3:CD28 ratio of antibody bound to beads may be used, a 1:50 CD3:CD28 ratio of antibody bound to beads may be used, a 1:30 CD3:CD28 ratio of antibody bound to beads may be used, a 1:10 CD3:CD28 ratio of antibody bound to beads may be used, or a 1:3 CD3:CD28 ratio of antibody bound to the beads may be used. Alternatively, a 3:1 CD3:CD28 ratio of antibody bound to the beads may be used.

Ratios of particles to cells may range from 1:500 to 500:1 and any integer values in between may be used to stimulate T cells or other target cells. As those of ordinary skill in the art can readily appreciate, the ratio of particles to cells may depend on particle size relative to the target cell. For example, small sized beads may only bind a few cells, while larger beads may bind many cells. The ratio of anti-CD3- and anti-CD28-coupled particles to T cells that result in T cell stimulation may vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. Alternatively, a ratio of particles to cells of 1:1 or less may be used. A preferred particle:cell ratio may be 1:5. The ratio of particles to cells may be varied depending on the day of stimulation. For example, the ratio of particles to cells may be from 1:1 to 10:1 on the first day and additional particles may be added to the cells every day or every other day thereafter for up to 10 days, at final ratios of from 1:1 to 1:10 (based on cell counts on the day of addition). Alternatively, the ratio of particles to cells may be 1:1 on the first day of stimulation and adjusted to 1:5 on the third and fifth days of stimulation. In another instance, particles may be added on a daily or every other day basis to a final ratio of 1:1 on the first day, and 1:5 on the third and fifth days of stimulation. In another instance, the ratio of particles to cells may be 2:1 on the first day of stimulation and adjusted to 1:10 on the third and fifth days of stimulation. In another instance, particles may be added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

The T cells may be combined with agent-coated beads, the beads and the cells may be subsequently separated, and then the cells may be cultured. Alternatively, prior to culture, the agent-coated beads and cells may not be separated but may be cultured together. In a further instance, the beads and cells may first be concentrated by application of a force, such as a magnetic force, resulting in increased ligation of cell surface markers, thereby inducing cell stimulation.

Cells (for example, 10⁴ to 10⁹ T cells) and beads (for example, DYNABEADS® M-450 CD3/CD28 T paramagnetic beads at a ratio of 1:1) may be combined in a buffer, preferably PBS (without divalent cations such as, calcium and magnesium). Again, those of ordinary skill in the art can readily appreciate any cell concentration that may be used. It may be desirable to significantly decrease the volume in which particles and cells are mixed together (i.e., increase the concentration of cells), to ensure maximum contact of cells and particles. For example, a concentration of about 2 billion cells/mL may be used. In another instance, a concentration of more than 100 million cells/mL may be used. In a further instance, a concentration of cells of 10, 15, 20, 25, 30, 35, 40, 45, or 50 million cells/mL may be used. In yet another instance, a concentration of cells from 75, 80, 85, 90, 95, or 100 million cells/mL may be used. In further instances, concentrations of 125 or 150 million cells/mL may be used. Using high concentrations may result in increased cell yield, cell activation, and cell expansion. Further, use of high cell concentrations may allow more efficient capture of cells that may weakly express target antigens of interest, such as CD28-negative T cells. Such populations of cells may have therapeutic value and would be desirable to obtain in certain embodiments. For example, using high concentration of cells may allow more efficient selection of CD8+ T cells that normally have weaker CD28 expression.

The mixture may be cultured for several hours (about 3 hours) to about 14 days or any hourly integer value in between. The mixture may be cultured for 21 days. In one instance the beads and the T cells may be cultured together for about eight days. In another instance, the beads and T cells may be cultured together for 2-3 days. Several cycles of stimulation may also be desired such that culture time of T cells may be 60 days or more.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Conditions appropriate for lymphocyte culture include an appropriate media (e.g., Minimal Essential Media or RPMI Media 1640 or, X-vivo 15, (Lonza), CellGro media (Cellgenix), IMDM (Gibco)) that may contain factors necessary for proliferation and viability, including serum (e.g., fetal bovine, human or horse serum), interleukin-2 (IL-2), insulin, IFN-γ, IL-4, IL-7, GM-CSF, IL-10, IL-12, IL-15, IL-18, TGFβ, and TNF-α or any other additives for the growth of cells known to the skilled artisan. Other additives for the growth of cells may include, but are not limited to, surfactant, plasmanate, and reducing agents such as N-acetyl-cysteine and 2-mercaptoethanol. Media may include RPMI 1640, AIM-V, DMEM, MEM, α-MEM, F-12, X-Vivo 15, X-Vivo 20, IMDM and CellGro, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of NK cells and T cells. Antibiotics, e.g., penicillin and streptomycin, may be included only in experimental cultures, not in cultures of cells that are to be infused into a subject. The lymphocytes may be maintained under conditions necessary to support growth, for example, an appropriate temperature (e.g., 37° C.) and atmosphere (e.g., air plus 5% C02).

In a further embodiment, the invention relates to the method according to the invention, wherein the at least one synthetic polynucleotide is introduced into the lymphocyte by viral transduction, in particular by lentiviral transduction.

As described above, the synthetic polynucleotide(s) encoding the at least one IRP and, optionally, the CAR may be introduced into the lymphocyte by any method known in the art. However. It is preferred that the synthetic polynucleotide(s) is/are introduced into the lymphocyte by viral transduction, as described above. More preferably, the viral vector that is used for introducing synthetic polynucleotides into the lymphocyte is a lentiviral vector. Since viral vectors usually integrate into the host cell genome at a random position, it is preferred that the synthetic polynucleotide comprises at least one gene encoding an IRP and the regulatory elements that are required to express the at least one gene encoding the IRP in the host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

In one embodiment, the invention relates to the method according to the invention, wherein the synthetic polynucleotide encoding the CAR is transcriptionally linked to the synthetic polynucleotide encoding IRP1 and/or IRP2.

In one embodiment, the invention relates to the method according to the invention, wherein the synthetic polynucleotide encoding the CAR and the polynucleotide encoding IRP1 and/or IRP2 are linked by a polynucleotide encoding a self-cleaving peptide.

In one embodiment, the invention relates to the method according to the invention, wherein the self-cleaving peptide is a 2A self-cleaving peptide.

In one embodiment, the invention relates to the method according to the invention, wherein the self-cleaving peptide is T2A.

In one embodiment, the invention relates to the method according to the invention, wherein the one or more synthetic polynucleotide is introduced into the lymphocyte by viral transduction.

In one embodiment, the invention relates to the method according to the invention, wherein viral transduction is performed with a viral vector according to any of the embodiments provided herein.

Examples of the synthetic polynucleotides encoding IRP1 and/or IRP2 and, optionally, the CAR, a promoter and/or further regulatory elements (such as IRES or polynucleotides encoding self-cleaving peptides) are disclosed elsewhere herein and apply mutatis mutandis to the claimed method. Preferably, the viral vector according to the invention is used in the method according to the invention.

### BRIEF DESCRIPTION OF THE FIGURES

FIG. 1: Naive and cytokine-enhanced NK cells similarly rely on glycolysis for IFN-γ production

(A) Schematic of the experiment used to generate CE NK cells. (B) IFN-γ production by NV and CE NK cells unstimulated (no stim) or stimulated with IL-12/IL-18 (mean±SEM, n=18 donors). (C) GMFI of CD69 expression on NV and CE NK cells unstimulated (no stim) or stimulated with IL-12/IL-18 (mean±SEM, n=13 donors). (D) PCA of the transcriptome data, depicting the group relationships in NV and CE NK cells unstimulated (no stim) or stimulated with IL-12/IL-18. The proportion of component variance is indicated as percentage (n=5 donors). (E) Heatmap of relative expression of mRNA encoding for glycolysis genes from NV and CE NK cells unstimulated (no stim) or stimulated with IL-12/IL-18 of the transcriptome data (n=5 donors). (F) Upper panel: Representative mitochondrial perturbation assay of NV and CE NK cells unstimulated (no stim) or stimulated with IL-12/IL-18. Glycolysis (extracellular acidification rate—ECAR) was measured “in Seahorse” after injection of oligomycin, FCCP, and rotenone. Lower panel: Basal and maximal rate of ECAR in NV and CE NK cells unstimulated (no stim) or stimulated with IL-12/IL-18 analyzed by mitochondrial perturbation assay (mean±SEM, n=12 donors). (G) Upper panel: Representative histogram of NBDG uptake in NV and CE NK cells unstimulated (no stim) or stimulated with IL-12/IL-18. Lower panel: GMFI of NBDG uptake in NV and CE NK cells unstimulated (no stim) or stimulated with IL-12/IL-18 (mean±SEM, n=15 donors). (H) Expression of IFNG mRNA in NV and CE NK cells unstimulated (no stim) or stimulated with IL-12/IL-18, IL-12/IL-18+2-DG. Transcript levels were determined relative to 18S mRNA levels and normalized to unstimulated (no stim) NV NK cells (mean±SEM, n=6 donors). (I) Upper panel: IFN-γ production by NV and CE NK cells unstimulated (no stim) or stimulated with IL-12/IL-18, IL-12/IL-18+2-DG (mean±SEM, n=6 donors). Lower panel. IFN-γ production by NV and CE NK cells unstimulated (no stim) or stimulated with IL-12/IL-18 in 10 mM glucose and in 2 mM glucose (mean±SEM, n=5 donors). Statistical significance was assessed by paired two-tailed Student's t-test (C, F, H, I) or linear-regression analysis (B, G, H, 1). *p<0.05, **p<0.01, *** p<0.001, ns, not significant.

FIG. 2: Activated CE NK cells are characterized by high levels of cell-surface CD71 and rapid cell proliferation

(A) Upper panel: Representative histogram of CD98 expression on NV and CE NK cells unstimulated (no stim) or stimulated with IL-12/IL-18. Lower panel: MFI of CD98 expression on NV and CE NK cells unstimulated (no stim) or stimulated with IL-12/IL-18 (mean±SEM, n=8 donors). (B) Upper panel: Representative histogram of CD71 expression on NV and CE NK cells unstimulated (no stim) or stimulated with IL-12/IL-18. Lower panel. GMFI and percentage of CD71 expression on NV and CE NK cells unstimulated (no stim) or stimulated with IL-12/IL-18 (mean±SEM, n=14 donors). (C) Left panel: Representative Western blot of total CD71 expression in NV and CE NK cells unstimulated (no stim) or stimulated with IL-12/IL-18. Right panel: Total CD71 expression normalized to actin in NV and CE NK cells unstimulated (no stim) or stimulated with IL-12/IL-18 (mean & SEM, n=13 donors). (D) Left panel. GMFI of CD71 expression on NV and CE NK cells unstimulated (no stim) or stimulated with K562 (mean±SEM, n=6). Right panel: Percentage of CD71+NK cells on NV and CE NK cells unstimulated (no stim) or stimulated with K562 (mean±SEM, n=6 donors). (E) Upper panel: Representative histogram of Tf-488 uptake in NV and CE NK cells unstimulated (no stim) or stimulated with IL-12/IL-18. Lower panel: GMFI of Tf-488 uptake in NV and CE NK cells unstimulated (no stim) or stimulated with IL-12/IL-18 (mean±SEM, n=10 donors). (F) Upper panel: Schematic of the experiment used to analyze CFSE dilution in NV and CE NK cells. Middle panel: Representative histogram of CFSE dilution in NV and CE NK cells unstimulated (no stim) or stimulated with IL-12/IL-18. Lower panel: Percentage of proliferated NV and CE NK cells unstimulated (no stim) or stimulated with IL-12/IL-18 analyzed by CFSE dilution (mean±SEM, n=13 donors). (G) Heatmap of relative expression of mRNA encoding for cell cycle genes (GO:0006098) in NV and CE NK cells unstimulated (no stim) or stimulated with IL-12/IL-18 (n=5 donors). (H) Percentage of proliferated NV and CE NK cells unstimulated (no stim) or stimulated with IL-12/IL-18, IL-12/IL-18+BIP (1, 10 and 50 μM) analyzed by CFSE dilution (mean±SEM, n=11 donors for unstimulated, IL-12/IL-18 and IL-12/IL-18+BIP 10 μM stimulation, n=8 donors for IL-12/IL-18+BIP 1 μM stimulation, n=3 donors IL-12/IL-18+BIP 50 μM stimulation). (I) GMFI of CD69 expression on NV and CE NK cells unstimulated (no stim) or stimulated with IL-12/IL-18, IL-12/IL-18+BIP 100 μM (mean±SEM, n=5 donors). (J) Upper panel: Heatmap of relative expression of mRNA encoding for PPP genes (GO:0006098) in NV and CE NK cells unstimulated (no stim) or stimulated with IL-12/IL-18 (n=5 donors). Lower panel: Percentage of proliferated cells in NV and CE NK cells unstimulated (no stim) or stimulated with IL-12/IL-18, IL-12/IL-18+6AN 50 μM analyzed by CFSE dilution (mean f SEM, n=6 donors). Statistical significance was assessed by paired two-tailed Student's t-test (F, H, I, J) or linear-regression analysis (A, B, C, E). *p<0.05, **p<0.01, *** p<0.001, ns, not significant.

FIG. 3: CD71-mediated iron uptake and dietary iron availability impact NK cell function (A) Upper panel: Schematic of the experiment used to analyze CFSE dilution in WT and TfrcY20H/Y20H NK cells from spleen. Lower left panel: Representative histogram of CFSE dilution in WT and TfrcY20H/Y20H NK1.1+NK cells from spleen with IL-12/IL-18 stimulation. Lower right panel: Percentage of proliferated WT and TfrcY20H/Y20H NK1.1+NK cells from spleen in IL-15 LD or stimulated with IL-12/IL-18 analyzed by CFSE dilution (mean±SEM, n=5 for WT NK cells, n=6 for TfrcY20H/Y20H NK cells). (B) Upper panel: Schematic of MCMV infection experiment of mice fed +/− iron feed for 6 weeks. Lower panel: Serum levels of iron, ferritin, UIBC, TIBC; and hematocrit from mice fed +/− iron feed for 6 weeks (mean±SEM, n=8-18 for iron, ferritin, UIBC and TIBC and n=3 for hematocrit). (C) Left panel. Percentage of NK1.1+NK cells in spleen of mice fed +/− iron feed for 6 weeks (mean±SEM, n=5). Right panel: Percentage of CD8+, CD4+, CD19+ cells in spleen of mice fed +/− iron feed for 6 weeks (mean±SEM, n=5). (D) Left panel: Percentage of CD27+CD 11b−, CD27+CD 11b+, CD27-CD11b+ on NK1.1+NK cells in spleen of mice fed +/− iron feed for 6 weeks (mean±SEM, n=5). Right panel: Percentage of KLRG1+ and CD62L+ on NK1.1+NK cells in spleen of mice fed +/− iron feed for 6 weeks (mean±SEM, n=5). (E) Left panel: Viral titer in liver and spleen of WT MCMV-infected mice fed +/− iron feed for 6 weeks 3 dpi (each dot represents data from cells isolated from one mouse, data displayed as fold change difference normalized to mice fed + iron feed, horizontal line indicates median, n=10). Right panel: Viral titer in liver and spleen of Δm157 MCMV infected-mice fed an +/− iron feed for 6 weeks 3 dpi. (each dot represents data from cells isolated from one mouse, data displayed as fold change difference normalized to mice fed + iron feed, horizontal line indicates median, n=9-10). (F) Percentage of IFN-Y+ in NK1.1+NK cells in liver and spleen of WT MCMV-infected mice fed +/−feed for 6 weeks 1.5 dpi (mean f SEM, n=4-5). Statistical significance was assessed by unpaired two-tailed Student's t-test (A, B, C, D, E, F). *p<0.05, **p<0.01, *** p<0.001, **** p<0.0001, ns, not significant.

FIG. 4: CD71 supports NK cell proliferation and optimal effector function during viral infection

(A) Left panel: Percentage and absolute numbers of NK1.1+NK cells in liver of Tfrcfl/fl and Tfrcfl/flNcr1Cre mice (mean±SEM, n=10). Right panel: Percentage and absolute numbers of NK1.1+NK cells in spleen of Tfrcfl/fl and Tfrcfl/flNcr1Cre mice (mean±SEM, n=17-22). (B) Upper left panel: Percentage and absolute numbers of CD8+ cells in liver of Tfrcfl/fl and Tfrcfl/flNcr1Cre mice (mean±SEM, n=10). Upper right panel: Percentage and absolute numbers of CD4+ cells in liver of Tfrcfl/fl and Tfrcfl/flNcr1Cre mice (mean±SEM, n=4). Lower panel: Percentage and absolute numbers of CD19+ cells in liver of Tfrcfl/fl and Tfrcfl/flNcr1Cre mice (mean±SEM, n=10). (C) Upper left panel: Percentage and absolute numbers of CD8+ cells in spleen of Tfrcfl/fl and Tfrcfl/flNcr1Cre mice (mean±SEM, n=9-19). Upper right panel: Percentage and absolute numbers of CD4+ cells in spleen of Tfrcfl/fl and Tfrcfl/flNcr1Cre mice (mean±SEM, n=11-22). Lower panel: Percentage and absolute numbers of CD19+ cells in spleen of Tfrcf/f and Tfrcfl/flNcr1Cre mice (mean±SEM, n=12-22). (D) Upper panel: Percentage of CD27+CD11b−, CD27+CD11b+, CD27-CD11b+ on NK1.1+ NK cells in liver of Tfrcfl/fl and Tfrcfl/flNcr1Cre mice (mean±SEM, n=10). Lower panel. Percentage of CD62L+ and Ly6C+ on NK1.1+NK cells in liver of Tfrcfl/fl and Tfrcfl/flNcr1Cre mice (mean±SEM, n=6). (E) Upper panel: Percentage of CD27+CD11b−, CD27+CD11b+, CD27-CD11b+ on NK1.1+NK cells in spleen of Tfrcfl/fl and Tfrcfl/flNcr1Cre mice (mean±SEM, n=5). Lower panel: Percentage of KLRG1+, CD62L+ and Ly6C+ on NK1.1+NK cells in spleen of Tfrcfl/fl and Tfrcfl/flNcr1Cre mice (mean±SEM, n=5). (F) Percentage of Ly49H+ on NK1.1+NK cells in liver and spleen of Tfrcfl/fl and Tfrcfl/flNcr1Cre mice (mean±SEM, n=5-6). (G) Upper left panel: Schematic of adoptive transfer experiment into Klra8−/− recipients to track expansion of WT and Tfrcfl/fl NK cells upon MCMV infection. Upper right panel: Representative flow plot gated on adoptively transferred CD45.1+ and CD45.2+(Ly49H+NK1.1+) NK cells in liver of WT MCMV-infected recipients 7 dpi. Lower panel: Percentage of adoptively transferred WT (Ly49H+NK1.1+CD45.1+) and Tfrcfl/flNcr1Cre (Ly49H+NK1.1+CD45.2+) NK cells in liver, spleen, lung and blood of WT MCMV-infected recipients 7 and 30 dpi (each dot represents data from cells isolated from one mouse, bars indicate t SEM, two independent experiments, 1st n=5 for 7 dpi and n=3-5 for 30 dpi, 2nd n=4 for 7 dpi and n=2 for 30 dpi). (H) Left panel: Schematic of adoptive transfer experiment into Rag2−/−IL2rg−/− recipients to track expansion of WT and Tfrcfl/fl NK cells. Right panel: Percentage of adoptively transferred WT (Ly49H+NK1.1+CD45.1+) and Tfrcfl/flNcr1Cre (Ly49H+NK1.1+CD45.2+) NK cells in liver, spleen, lung and blood 6 dpt (each dot represents data from cells isolated from one mouse, bars indicate±SEM, n=3-4). (1) Upper left panel: Schematic of adoptive transfer experiment into Klra8−/− recipients to analyze CFSE dilution in WT and Tfrcfl/fl NK cells upon WT MCMV infection. Upper right panel: Representative histogram of CFSE dilution in adoptively transferred WT and Tfrcfl/flNcr1Cre NK1.1+NK cells in liver of WT MCMV-infected recipients 3.5 dpi. Lower panel: GMFI of CFSE of adoptively transferred WT (Ly49H+NK1.1+CD45.1+) and Tfrcfl/flNcr1Cre (Ly49H+NK1.1+CD45.2+) NK cells in liver and spleen of WT MCMV-infected recipients 3.5 dpi (mean±SEM, two independent experiments, 1st n=5, 2nd n=4). (J) Percentage of proliferated Tfrcfl/fl and Tfrcfl/flNcr1Cre NK1.1+NK cells from spleen in IL-15 LD or stimulated with IL-12/IL-18 analyzed by CFSE dilution (mean±SEM, n=3-4). (K) Upper panel: Schematic of MCMV infection experiment of Tfrc/fl and Tfrcfl/flNcr1cre mice. Middle panel: Percentage and absolute number of NK1.1+NK cells in liver of WT MCMV-infected Tfrcfl/fl and Tfrcfl/flNcr1cre mice 3.5 and 5.5 dpi (mean±SEM, n=4-6). Lower panel: Percentage and absolute number of NK1.1+NK cells in spleen of WT MCMV-infected Tfrcfl/fl and Tfrc/flNcr1cre mice 3.5 and 5.5 dpi (mean±SEM, n=3-6). (L) Viral titer in liver and spleen of WT MCMV-infected Tfrcfl/fl and Tfrcfl/flNcr1cre mice 3.5 dpi (each dot represents data from cells isolated from one mouse, horizontal line indicates median, n=5). (M) Percentage of IFN-γ+ in NK 1.1+NK cells in liver and spleen of WT MCMV-infected Tfrcfl/fl and Tfrcfl/flNcr1cre mice 1.5 dpi (mean±SEM, n=4). (N) Left panel: Percentage of CD27+CD11b−, CD27+CD11b+, CD27-CD11b+ on NK1.1+NK cells in spleen of WT MCMV-infected Tfrcfl/fl and Tfrcfl/flNcr1Cre mice 5.5 dpi (mean±SEM, n=3-5). Right panel: Percentage of KLRG1+ on NK1.1+NK cells in spleen of WT MCMV-infected Tfrcfl/fl and Tfrcfl/flNcr1Cre mice 5.5 dpi (mean±SEM, n=3-5). Statistical significance was assessed by unpaired two-tailed Student's t-test (A, B, C, D, E, F, I, J, K, L, M, N). *p<0.05, **p<0.01, *** p<0.001, **** p<0.0001, ns, not significant.

FIG. 5: Glycolysis is required for induction of CD71 in activated NK cells (A) Upper panel: Representative Western blot of total CD71 expression in NV and CE NK cells unstimulated (no stim) or stimulated with IL-12/IL-18, IL-12/IL-18+ActD (1 and 10 μM) and IL-12/IL-18+CHX (10 and 100 μg/ml). Lower left panel: Total CD71 expression normalized to actin in NV and CE NK cells unstimulated (no stim) or stimulated with IL-12/IL-18, IL-12/IL-18+ActD 10 μM (mean±SEM, n=3 donors). Lower right panel: Total CD71 expression normalized to actin in NV and CE NK cells unstimulated (no stim) or stimulated with IL-12/IL-18, IL-12/IL-18+CHX 100 μg/ml (mean±SEM, n=2 donors). (B) Expression of TFRC mRNA in NV and CE NK cells unstimulated (no stim) or stimulated with IL-12/IL-18, IL-12/IL-18+2-DG. Transcript levels were determined relative to 18S mRNA levels and normalized to unstimulated (no stim) NV NK cells (mean±SEM, n=6 donors). (C) Upper left panel: GMFI of CD71 expression on NV and CE NK cells unstimulated (no stim) or stimulated with IL-12/IL-18, IL-12/IL-18+2-DG (mean±SEM, n=6 donors). Upper right panel: Representative Western blot of total CD71 expression in NV and CE NK cells unstimulated (no stim) or stimulated with TL-12/IL-18, TL-12/IL-18+2-DG. Lower panel: Total CD71 expression normalized to actin in NV and CE NK cells unstimulated (no stim) or stimulated with IL-12/IL-18, IL-12/IL-18+2DG (mean±SEM, n=5 donors). (D) GMFI of CD71 expression on NV and CE NK cells unstimulated (no stim) or stimulated with IL-12/IL-18 in 10 mM glucose and in 2 mM glucose (mean±SEM, n=5 donors). (E) GMFI of Tf-488 uptake in NV and CE NK cells unstimulated (no stim) or stimulated with IL-12/IL-18, IL-12/IL-18+2-DG (mean±SEM, n=5 donors). (F) Upper panel: Representative Western blot of total c-Myc expression in NV and CE NK cells unstimulated (no stim) or stimulated with IL-12/IL-18. Lower panel: Total c-Myc expression normalized to actin in NV and CE NK cells unstimulated (no stim) or stimulated with IL-12/IL-18 (mean±SEM, n=6 donors). Statistical significance was assessed by paired two-tailed Student's t-test (A, B, C, D, E, F) or linear-regression analysis (B, C, E). *p<0.05, **p<0.01, *** p<0.001, ns, not significant.

FIG. 6: Cytokine priming induces the IRP/IRE regulatory system

(A) Upper panel: Expression of ACO1 and IREB2 mRNA in NV and CE NK cells unstimulated (no stim) or stimulated with IL-12/IL-18 of transcriptome data (n=5 donors). Middle panel: Representative Western blot of total IRP1 and IRP2 expression in NV and CE NK cells unstimulated (no stim) or stimulated with IL-12/IL-18. Lower panel. Total IRP1 and IRP2 expression normalized to actin in NV and CE NK cells unstimulated (no stim) or stimulated with IL-12/IL-18 (mean±SEM, n=7 donors for IRP1, n=6 donors for IRP2). (B) Heatmap of relative expression of mRNAs encoding for genes harboring IREs in NV and CE NK cells unstimulated (no stim) or stimulated with IL-12/IL-18 (n=5 donors). (C) Upper left panel. Expression of EIF4E mRNA in NV and CE NK cells unstimulated (no stim) or stimulated with IL-12/IL-18 of transcriptome data (n=5 donors). Upper right panel: Representative Western blot of total eIF4E expression in NV and CE NK cells unstimulated (no stim) or stimulated with IL-12/IL-18. Lower panel: Total eIF4E expression normalized to actin in NV and CE NK cells unstimulated (no stim) or stimulated with IL-12/IL-18 (mean t SEM, n=6 donors). (D) Upper panel: Representative histogram of HPG incorporation in NV and CE NK cells unstimulated (no stim) or stimulated with IL-12/IL-18. Lower panel: GMFI of HPG incorporation in NV and CE NK cells unstimulated (no stim) or stimulated with IL-12/IL-18 (mean±SEM, n=5 donors). (E) Left panel: Representative Western blot of total ferritin heavy chain 1 expression in NV and CE NK cells unstimulated (no stim) or stimulated with IL-12/IL-18. Right panel: Total ferritin heavy chain 1 expression normalized to actin in NV and CE NK cells unstimulated (no stim) or stimulated with IL-12/IL-18 (mean±SEM, n=4 donors). Statistical significance was assessed by paired two-tailed Student's t-test (A, C, E) or linear-regression analysis (D). *p<0.05, **p<0.01, ns, not significant.

FIG. 7: The IRP/ARE regulatory system orchestrates CD71 expression in NK cells (A) Expression of TFRC mRNA in NV and CE NK cells no stim vs. IL-12/IL-18, data derived from transcriptome data (n=5). (B) Expression of FTH1 mRNA in NV and CE NK cells no stim vs. IL-12/IL-18 of transcriptome data (n=5). (C) Representative Western blot of total IRP1 expression in NK92 cells transfected with control or Aco1 siRNA. Total IRP1 expression in NK92 cells transfected with control or Aco1 siRNA (n=7). (D) Representative Western blot of total IRP2 expression in NK92 cells transfected with control or IREB2 siRNA. Total IRP2 expression in NK92 cells transfected with control or IREB2 siRNA (n=6). (E) Left panel: Representative histogram of CD71 expression on NK92 cells transfected with control, ACO1 or IREB2 siRNA. Right panel: GMFI of CD71 expression on NK92 cells transfected with control, ACO1 or IREB2 siRNA (n=4-5). (F) Left panel: Representative Western blot of total FTH1 expression in NK92 cells transfected with control, Aco1 and IREB2 siRNA. Right panel: Total FTH1 expression in NK92 cells transfected with control, Aco1 and IREB2 siRNA (n=5). (G) Left panel: Representative Western blot of total IRP1 expression in NKL cells transfected with control or Aco1 siRNA. Right panel: Total IRP1 expression in NKL cells transfected with control or Aco1 siRNA (n=6). (H) Left panel: Representative Western blot of total IRP2 expression in NKL cells transfected with control or IREB2 siRNA. Right panel: Total IRP2 expression in NKL cells transfected with control or IREB2 siRNA (n=7). (1) Left panel: Representative histogram of CD71 expression on NKL cells transfected with control, ACO1 or IREB2 siRNA. Right panel: GMFI of CD71 expression on NKL cells transfected with control, ACO1 or IREB2 siRNA (n=5). (J) Left panel: Representative Western blot of total FTH1 expression in NKL cells transfected with control and Aco1 siRNA. Right panel: Total FTH1 expression in NKL cells transfected with control and Aco1 siRNA (n=6). (K) Left panel: Representative Western blot of total FTH1 expression in NKL cells transfected with control and IREB2 siRNA. Right panel: Total FTH1 expression in NKL cells transfected with control and IREB2 siRNA (n=5). All averaged data are presented as mean±s.e.m. and were analyzed using an unpaired two-tailed Student's t-test (a,b,d,e) or ANOWA (c). Asterisks indicate significance between groups. *p<0.05, **p <0.01, ns, not significant. (L) Left panel: Representative Western blot of total IRP2 expression in NK92 cells transfected with control or IREB2 sgRNA. Right panel: Total IRP2 expression in NK92 cells transfected with control or IREB2 sgRNA (n=3). (M) Left panel: GMFI of CD71 expression on NK92 cells transfected with control or IREB2 sgRNA (n=5). Right panel: Number of NK92 cells transfected with control or IREB2 sgRNA (n=4). All averaged data are presented as mean t s.e.m. and were analyzed using a two-tailed Student's t-test (A-B), unpaired two-tailed Student's t-test (C, D, G, H, J, K, L, M) or ANOWA (E, F, I). Asterisks indicate significance between groups. *p<0.05, **p<0.01, ***p<0.001, ns, not significant.

FIG. 8: Enforced IRP expression is a molecular module also supporting T cell proliferation. (A) Left panel: Representative Western blot of total IRP1 expression in Jurkat cells transfected with control or Aco1 siRNA. Right panel: Total IRP1 expression in Jurkat cells transfected with control or Aco1 siRNA (n=5). (B) Left panel: Representative Western blot of total IRP2 expression in Jurkat cells transfected with control or IREB2 siRNA. Right panel: Total IRP2 expression in Jurkat cells transfected with control or IREB2 siRNA (n=4). (C) Left panel: Representative histogram of CD71 expression on Jurkat cells transfected with control, ACO1 or IREB2 siRNA. Right panel: GMFI of CD71 expression on Jurkat cells transfected with control, ACO1 or IREB2 siRNA (n=7). (D) Left panel: Representative Western blot of total FTH1 expression in Jurkat cells transfected with control, Aco1 or IREB2 siRNA. Right panel: Total FTH1 expression in Jurkat cells transfected with control, Aco1 and IREB2 siRNA (n=5). (E) Representative Western blot of total IRP2 expression in IRP2 knockout (ko) Jurkat cells transduced with control vector coding for mCherry (LV-mCherry) or for IREB2 (LV-IREB2). (F) Upper panel: Representative histogram of CD71 expression on IRP2 ko Jurkat cells transduced with LV-mCherry or LV-IREB2. Lower left panel: GMFI of CD71 expression on IRP2 ko Jurkat cells transduced with LV-mCherry vs. LV-IREB2 (n=4). Lower right panel: Number of IRP2 ko Jurkat cells transduced with LV-mCherry vs. LV-IREB2 (n=3). (G) Left panel: Representative histogram of CD71 expression on primary CD4⁺ T cells transduced with LV-mCherry vs. LV-IREB2. Right panel: GMFI of CD71 expression on primary CD4⁺ T cells transduced with LV-mCherry vs. LV-IREB2 (n=2). (H) Left panel: Representative histogram of CD71 expression on primary CD8⁺ T cells transduced with LV-mCherry vs. LV-IREB2. Right panel: GMFI of CD71 expression on primary CD8⁺ T cells transduced with LV-mCherry vs. IREB2 (LV-IREB2) (n=2). (I) Representative Western blot of total IRP2 expression in untransduced CD4⁺ T cells (UTD), PSMA-specific CAR CD4 T cells (CAR) and PSMA-specific CAR CD4⁺ T cells co-expressing IRP2 (CAR-IREB2). (J) Upper panel: Representative histogram of CD71 expression on unstimulated UTD, CAR and CAR-IREB2 transduced CD4⁺ T cells. MFI of CD71 expression on unstimulated UTD, CAR and CAR-IREB2 transduced CD4⁺ T cells (n=3). Lower panel: Representative histogram of CD71 expression on Fab-stimulated UTD, CAR and CAR-IREB2 transduced CD4⁺ T cells. MFI of CD71 expression on Fab-stimulated UTD, CAR and CAR-IREB2 transduced CD4′ T cells (n=3). (K) Upper panel: Percentage of unstimulated UTD, CAR and CAR-IREB2 transduced CD4⁺ T cells having entered 0, 1 and 2 cycles of cell proliferation (n=3). Lower panel: Percentage of Fab-stimulated UTD, CAR and CAR-IREB2 transduced CD4⁺ T cells having entered 0, 1 and 2 cycles of cell proliferation (n=3). All averaged data are presented as mean±s.e.m. and were analyzed using an unpaired two-tailed Student's t-test (a,b,f) or ANOWA (c,d). Asterisks indicate significance between groups. *p<0.05, **p<0.01, ns, not significant.

### EXAMPLES

### Example 1: Naive and Cytokine-Enhanced NK Cells Similarly Rely on Glycolysis for IFN-γ Production

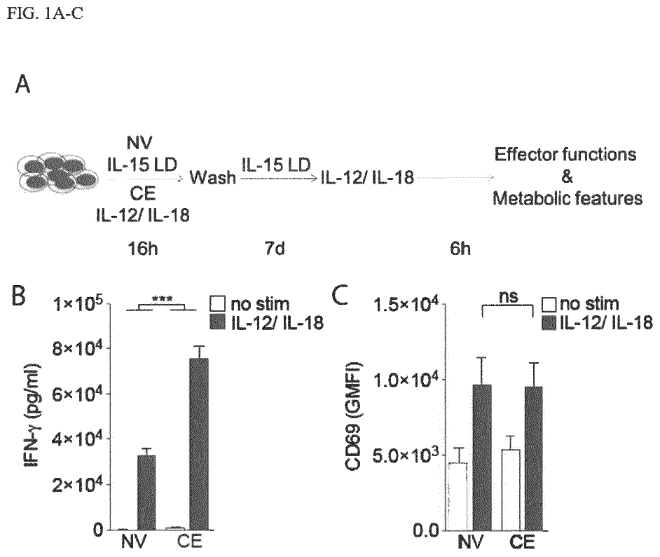

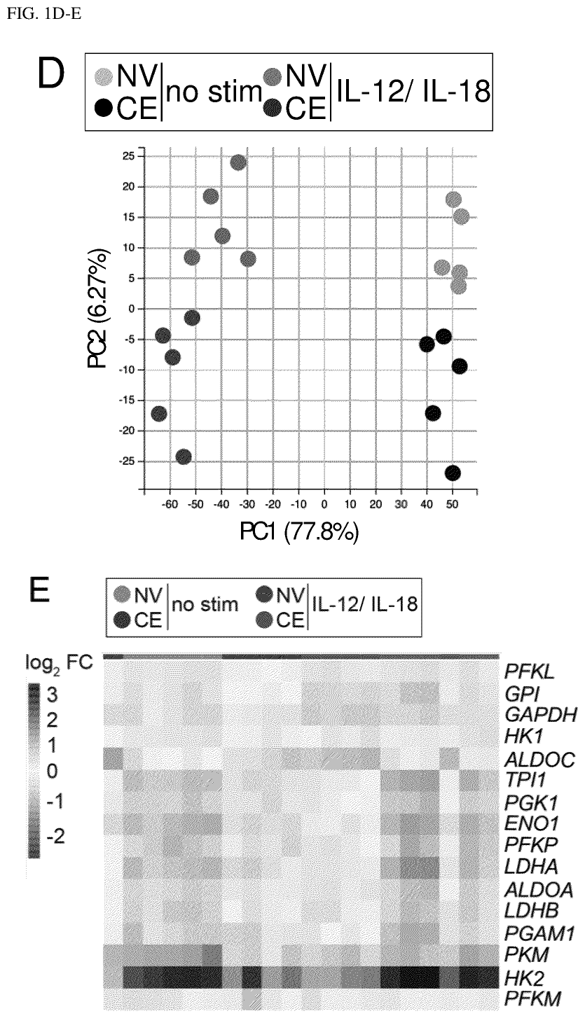

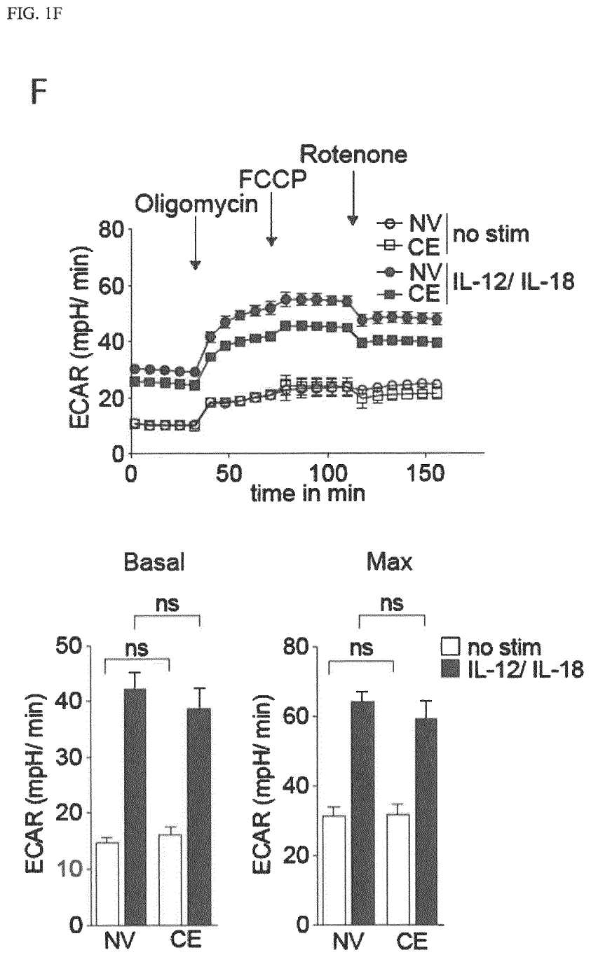

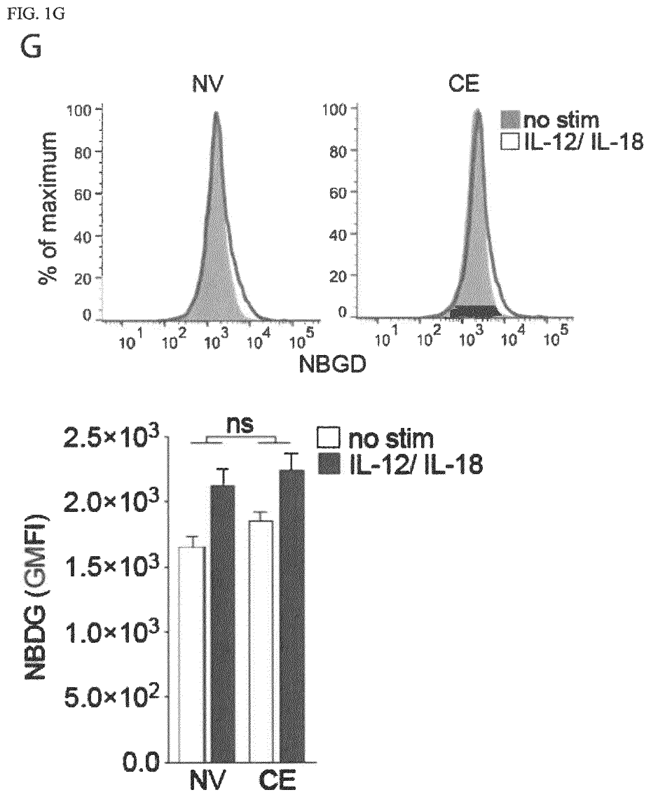

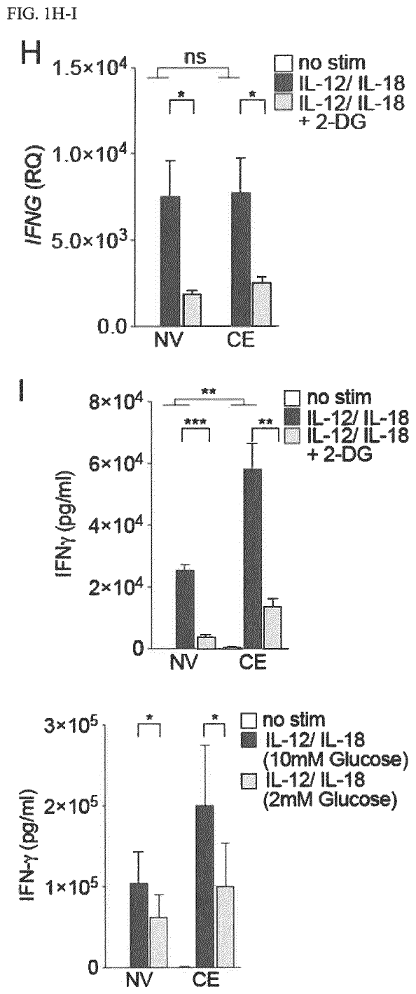

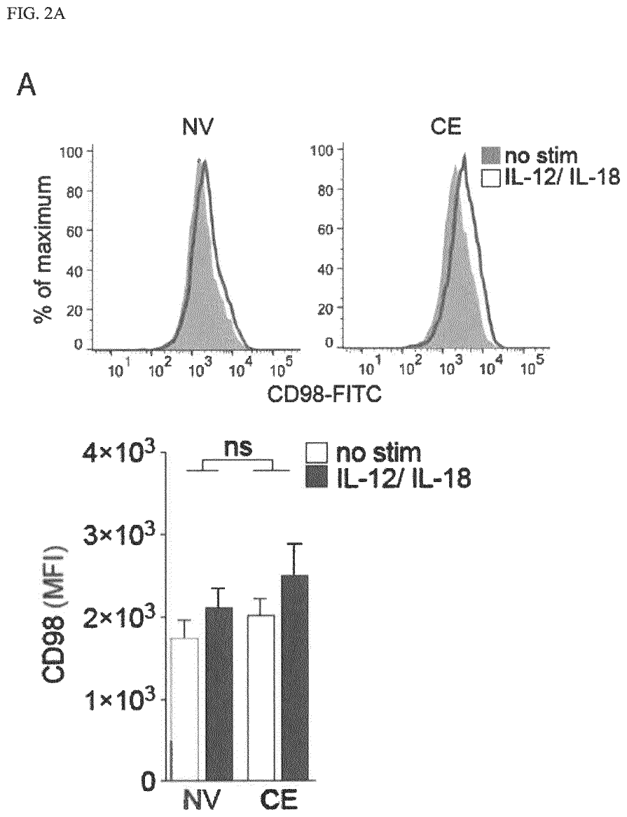

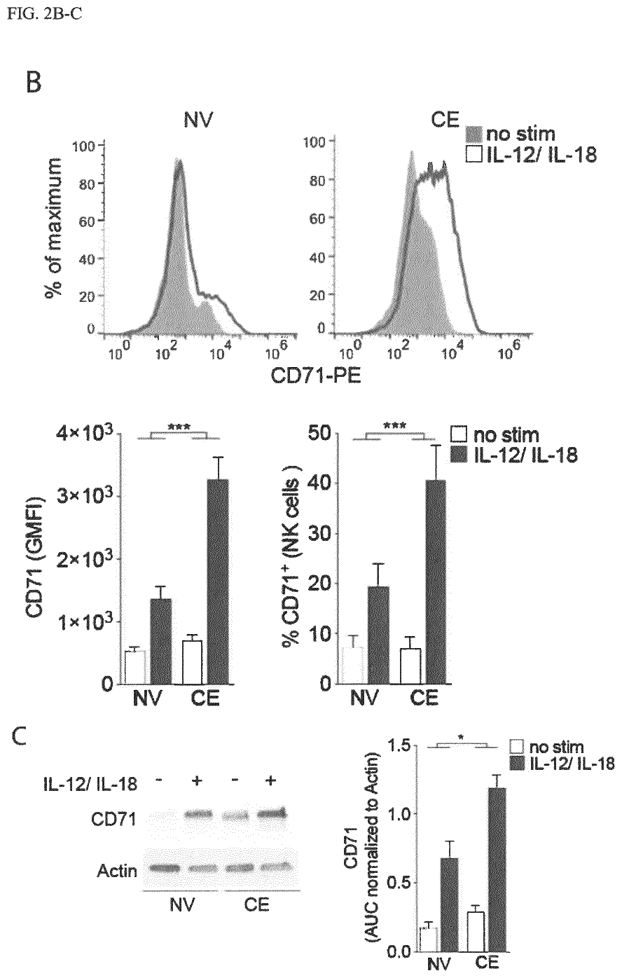

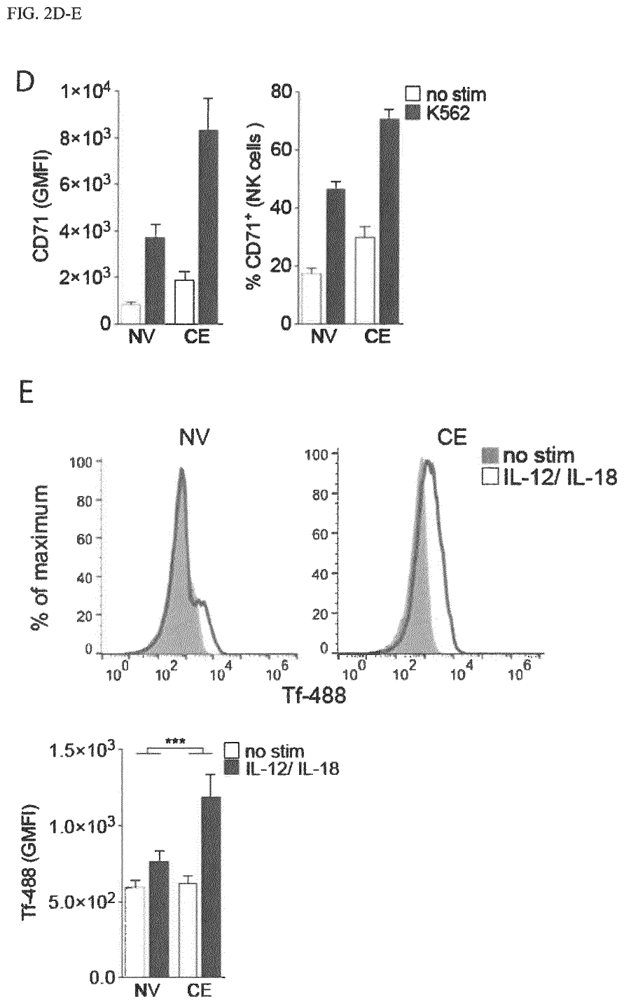

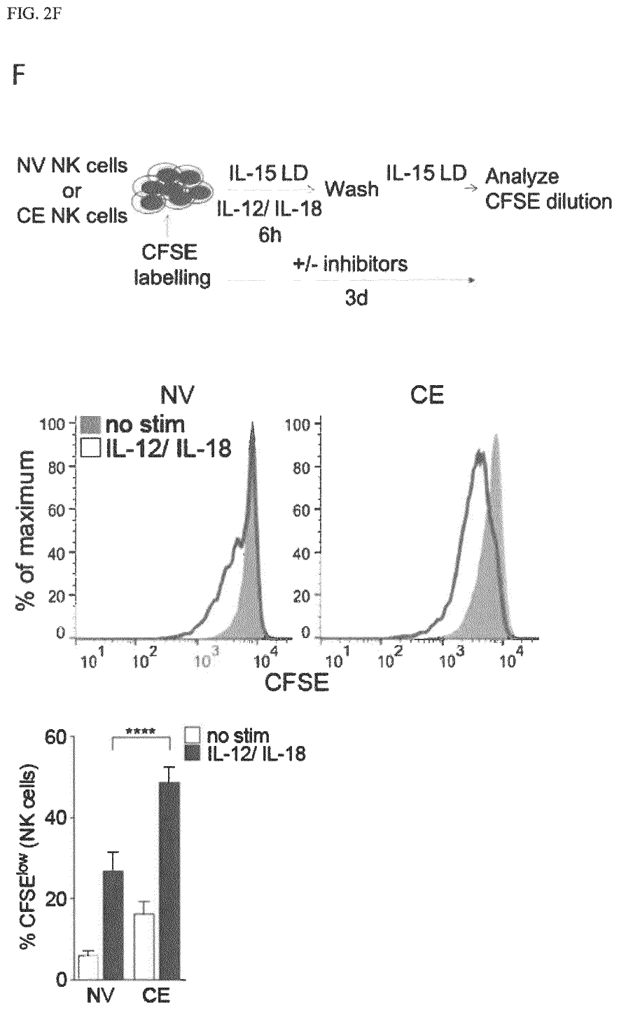

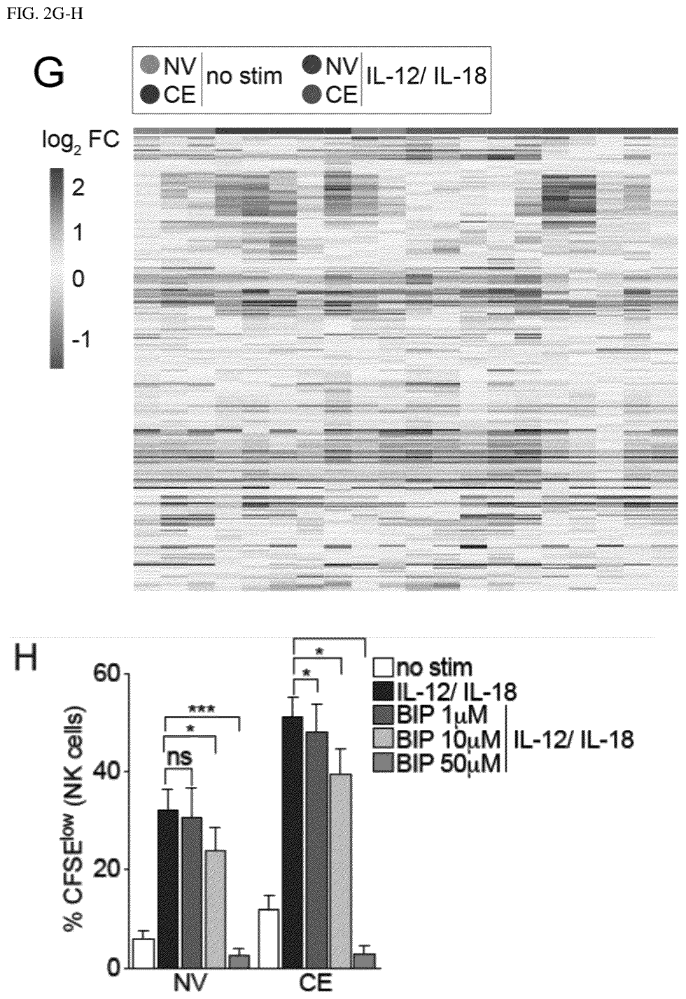

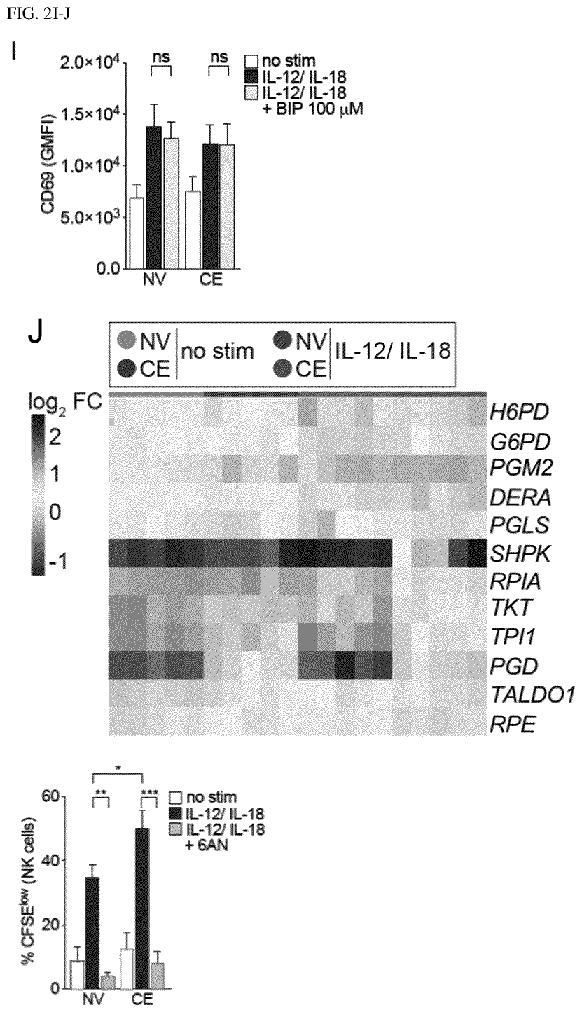

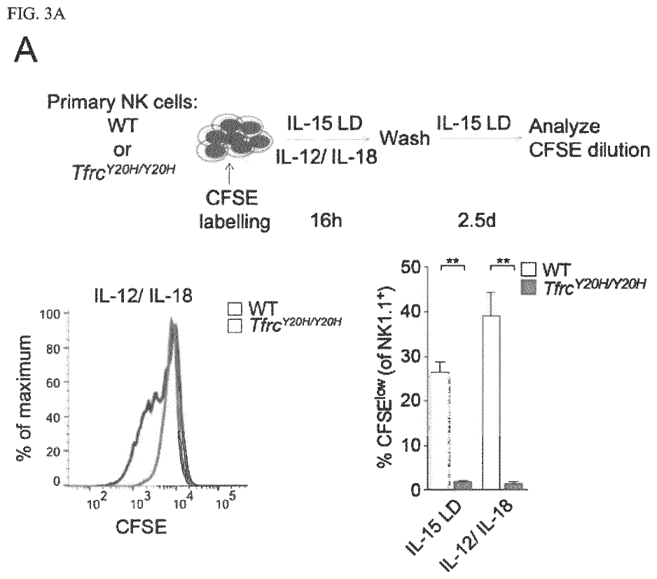

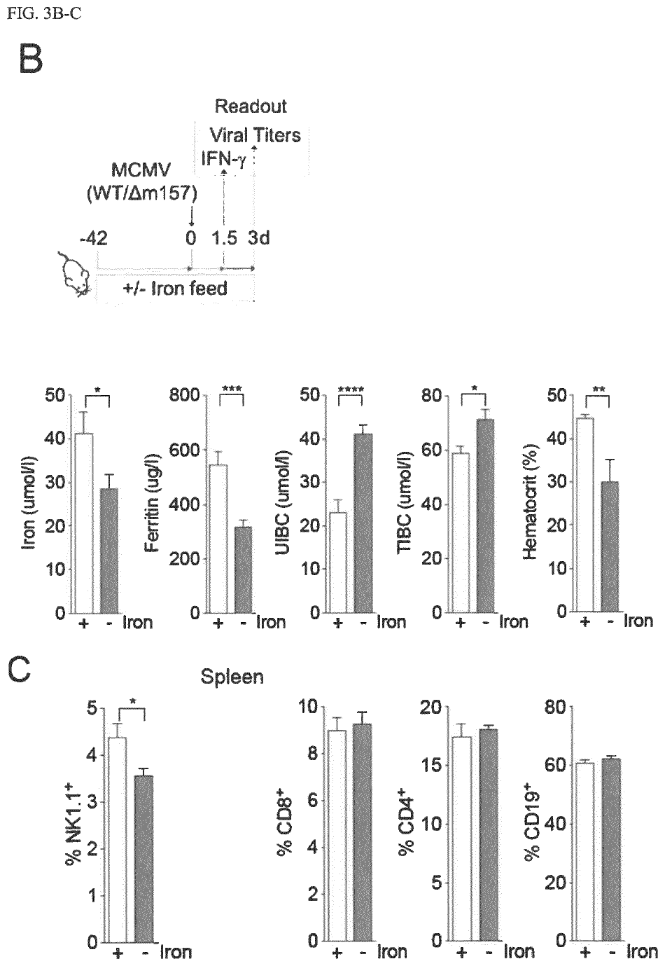

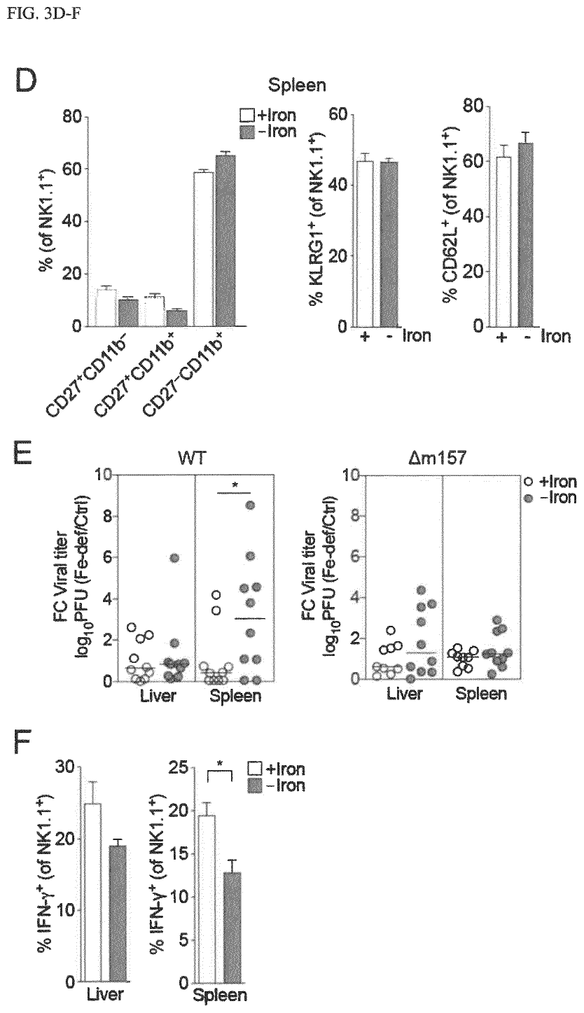

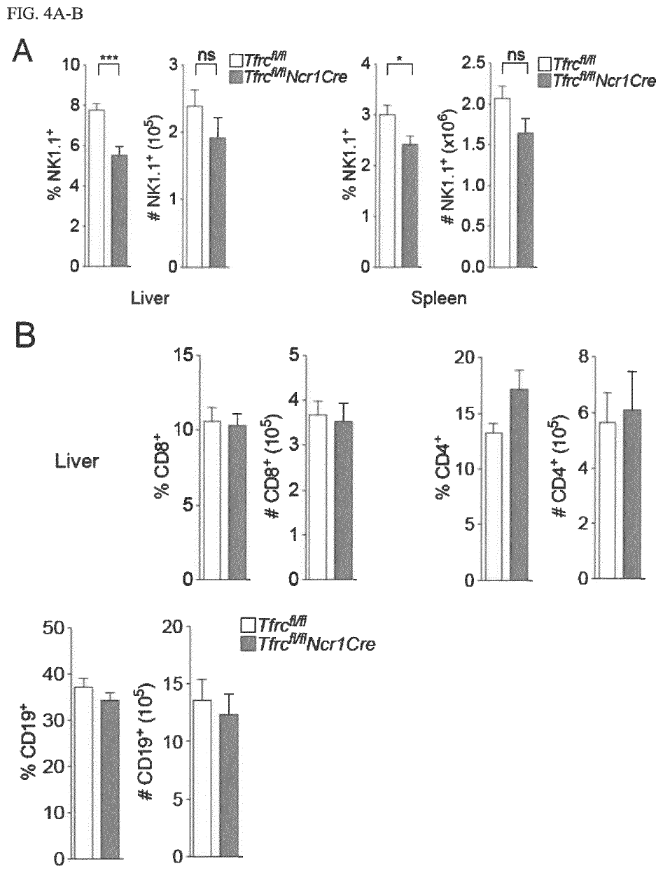

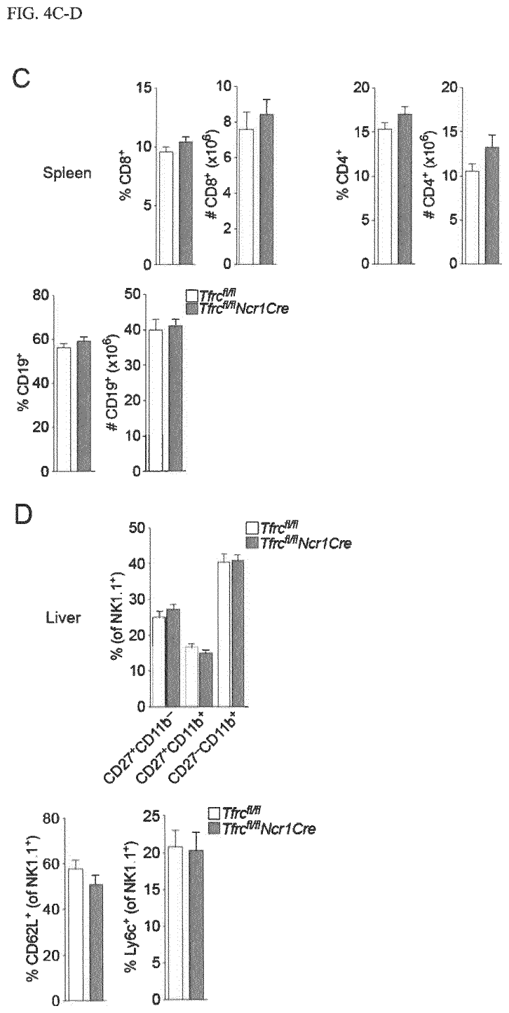

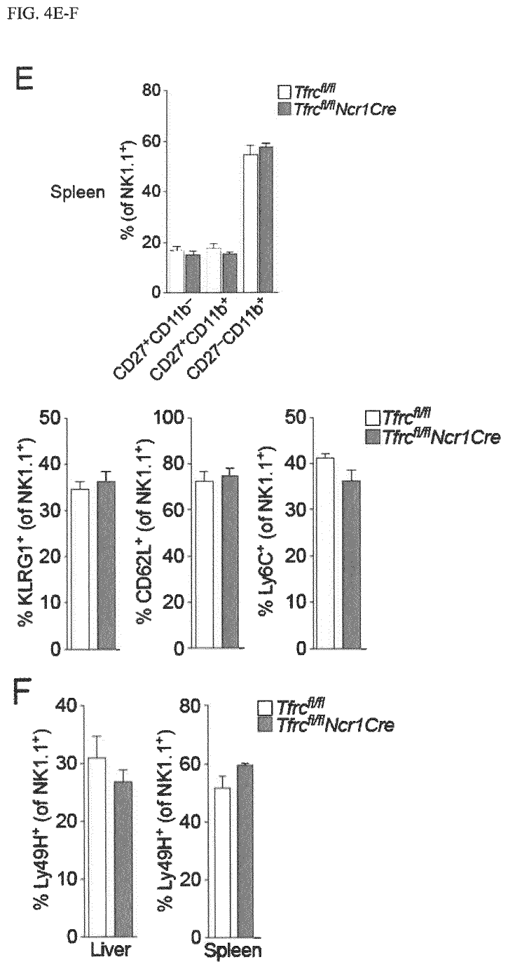

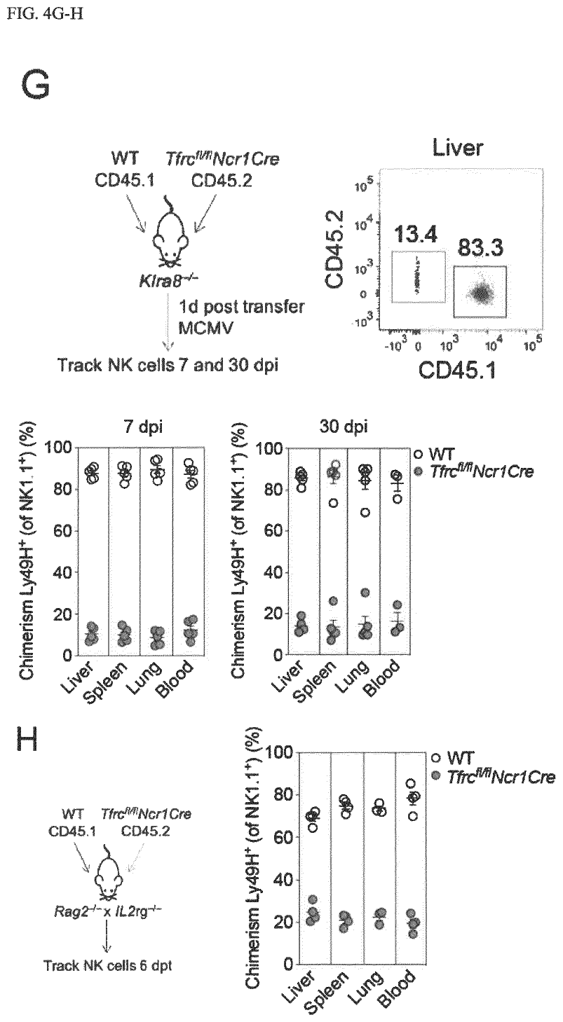

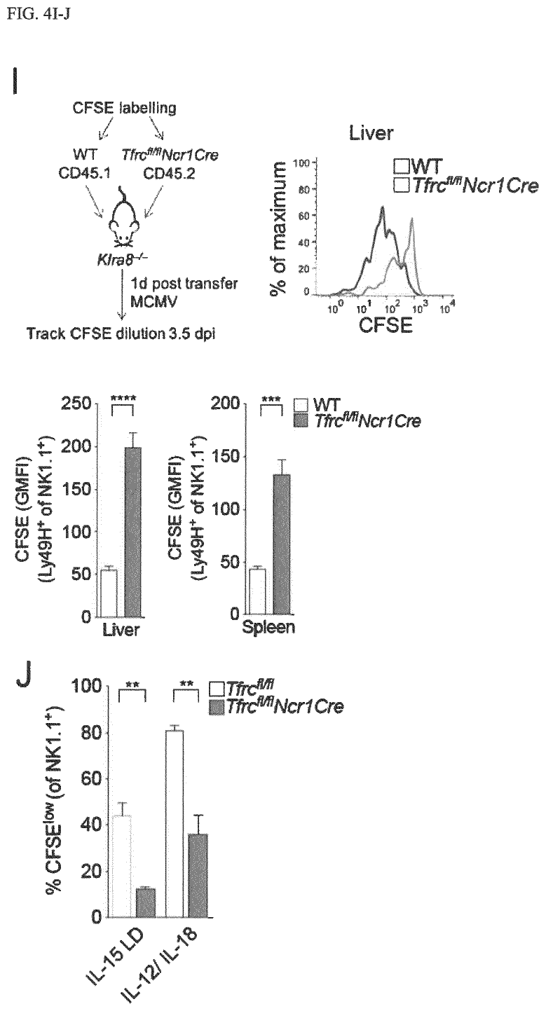

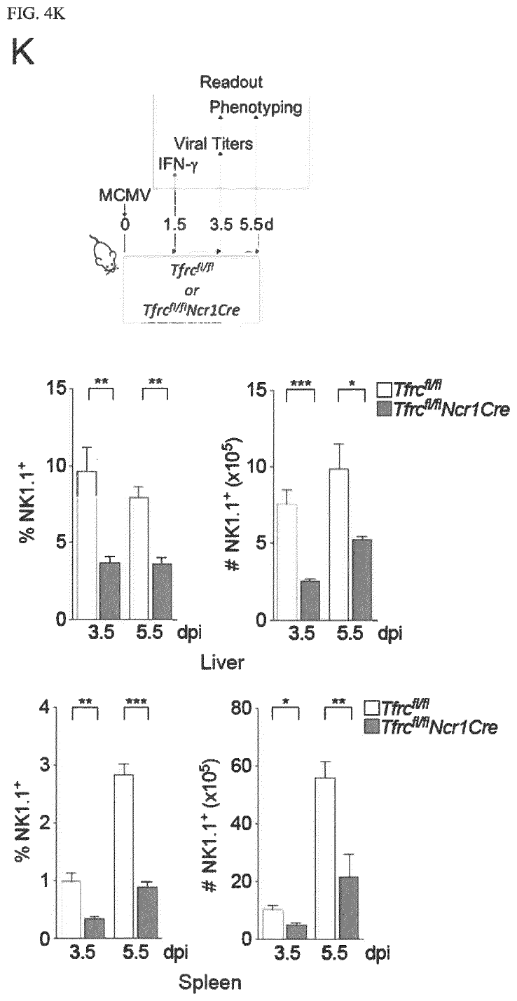

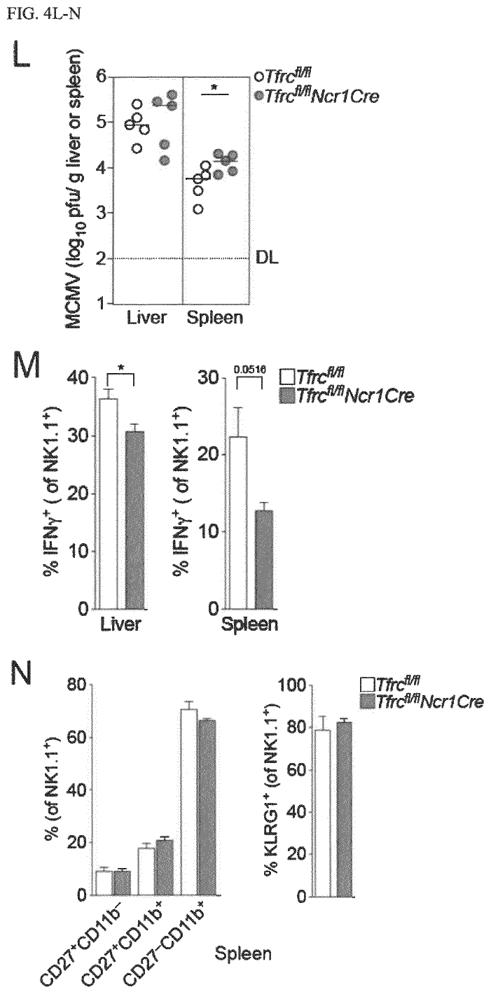

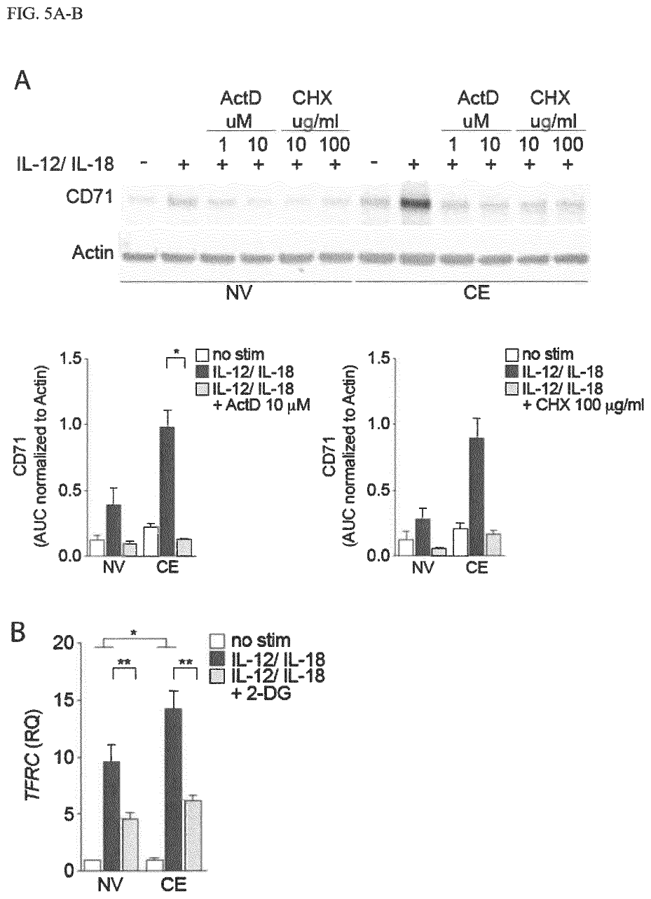

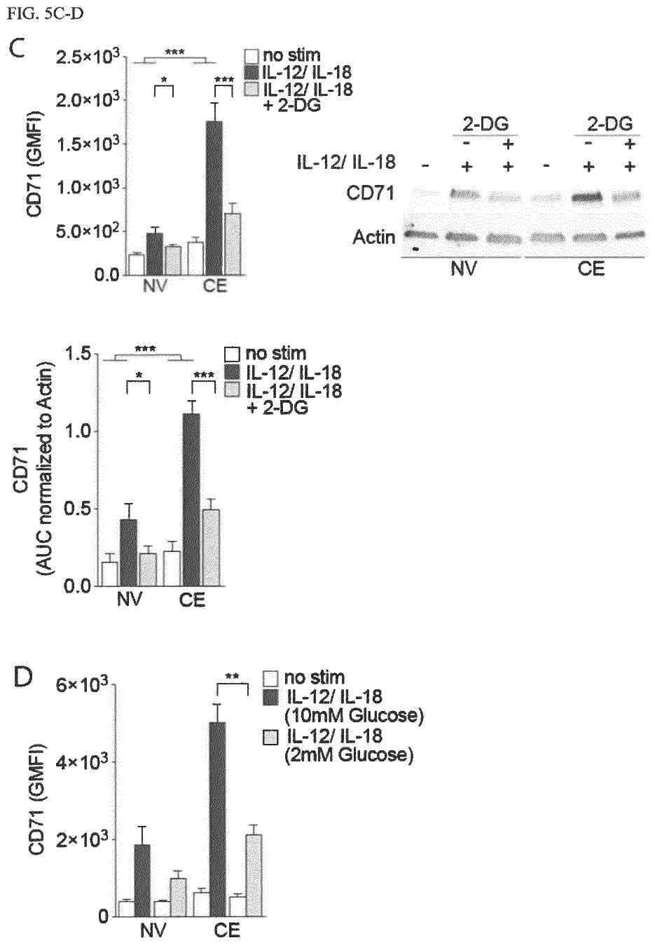

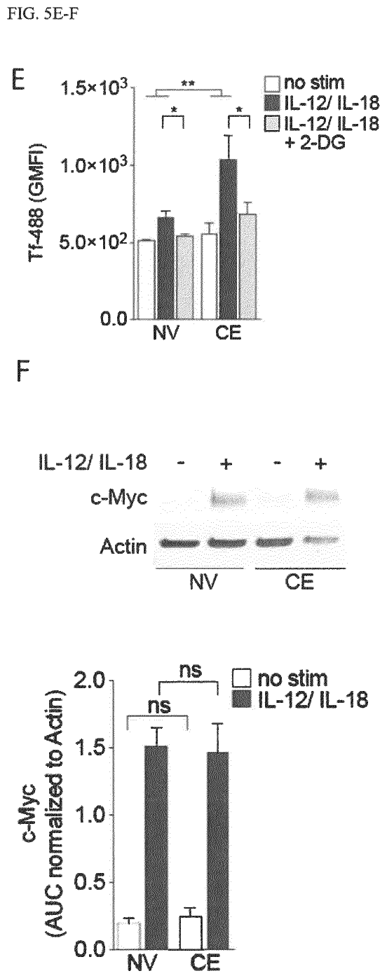

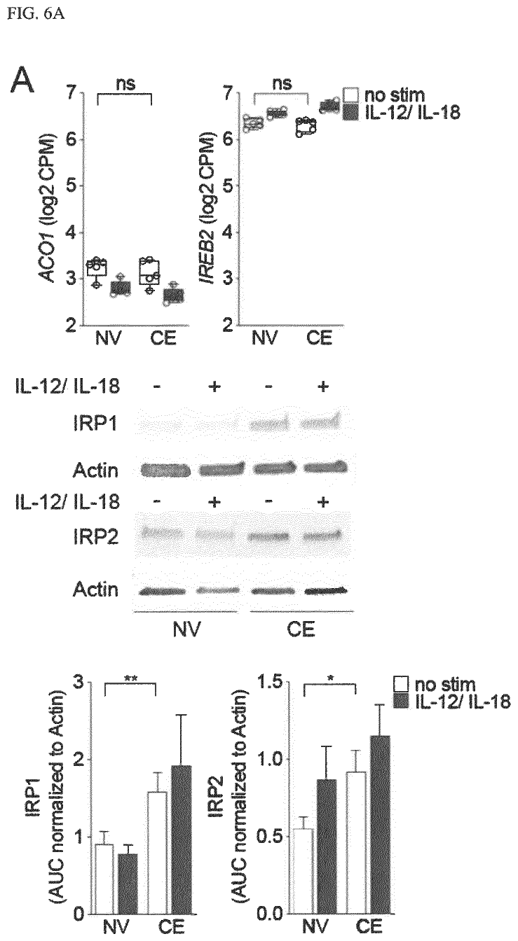

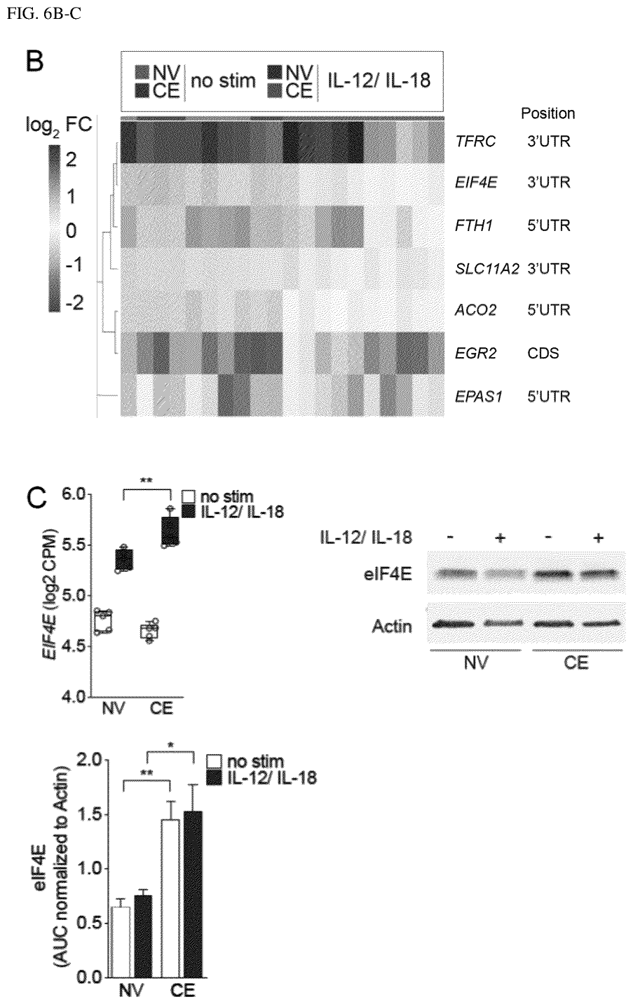

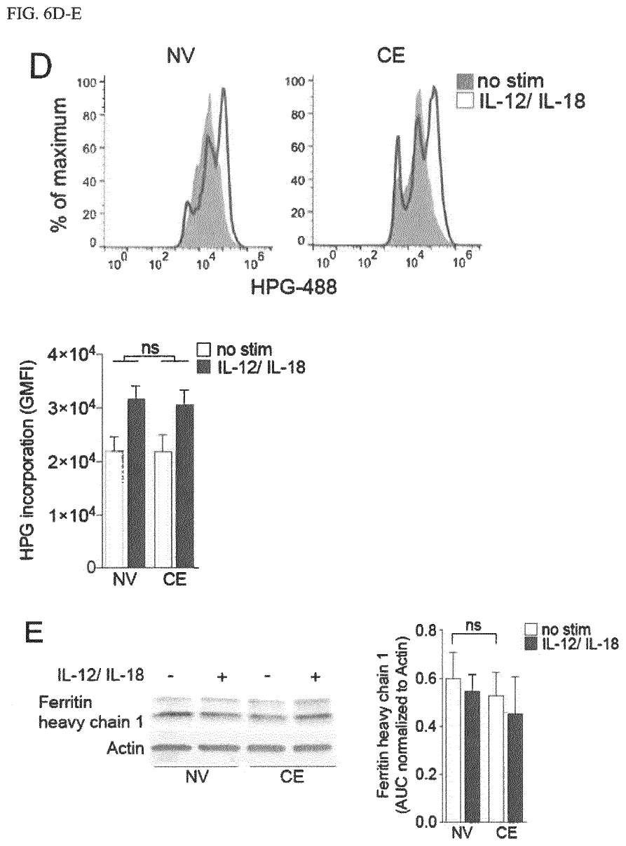

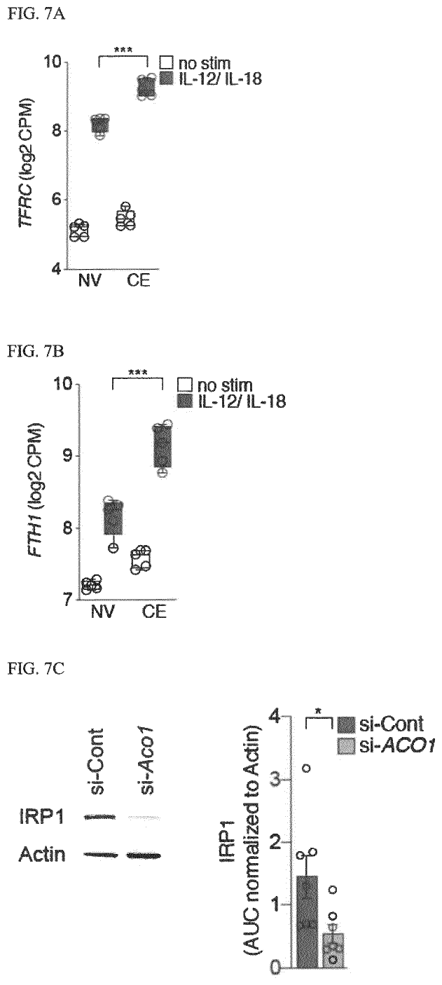

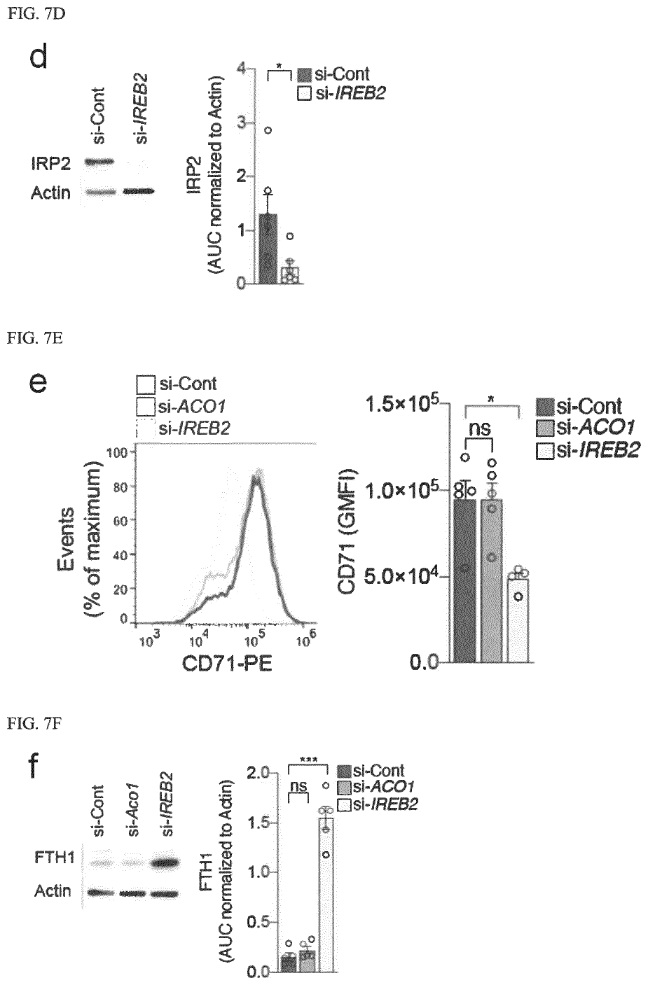

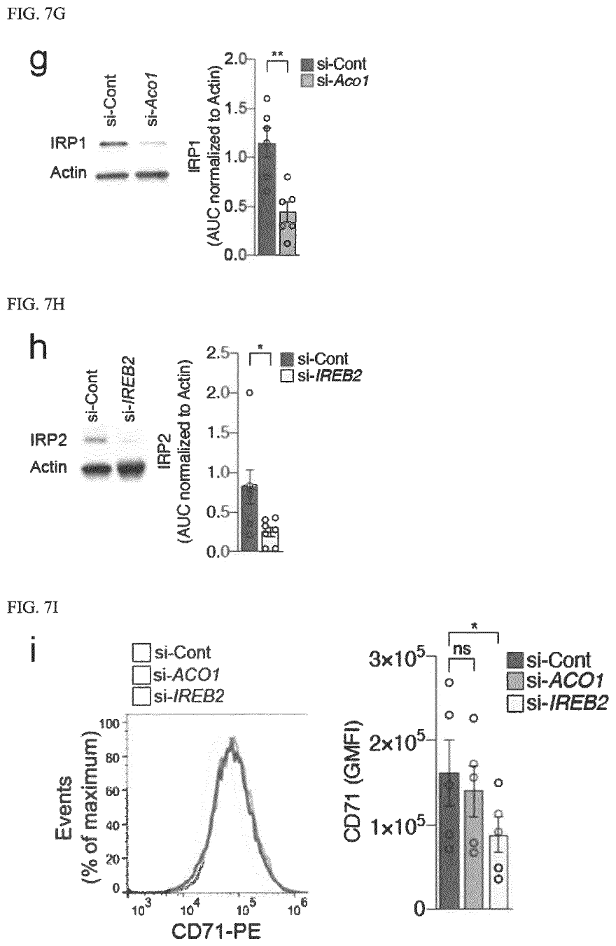

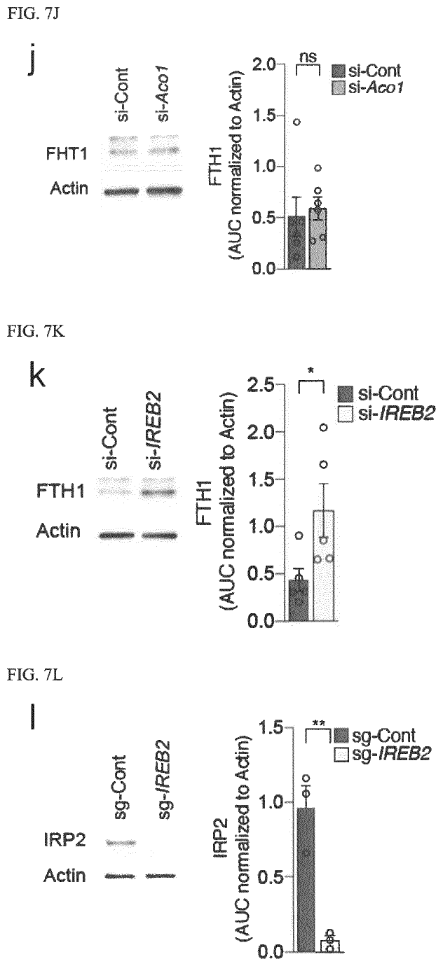

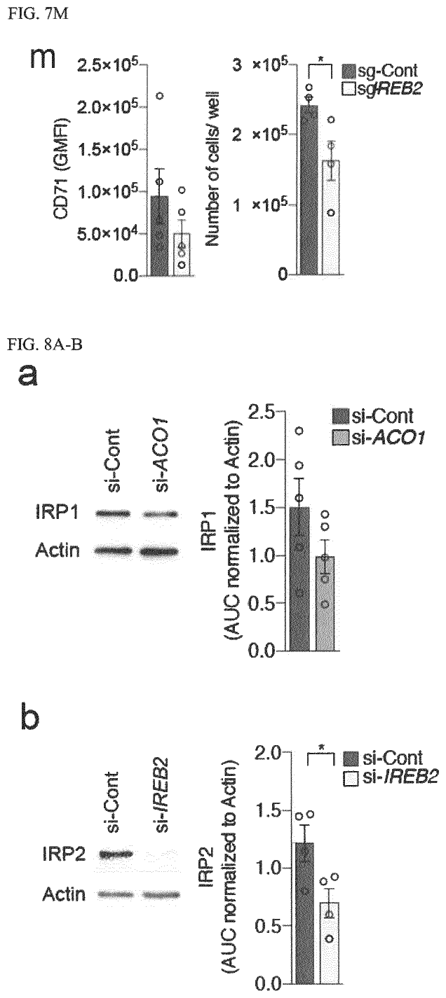

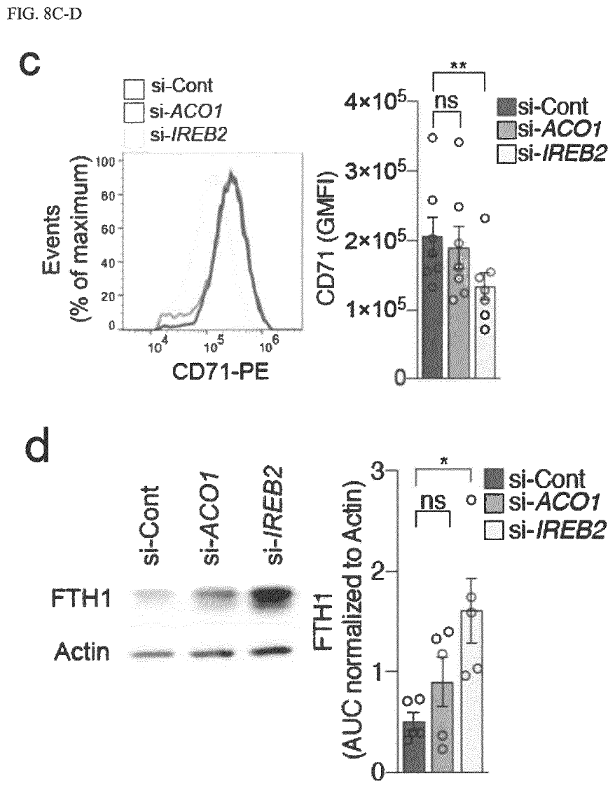

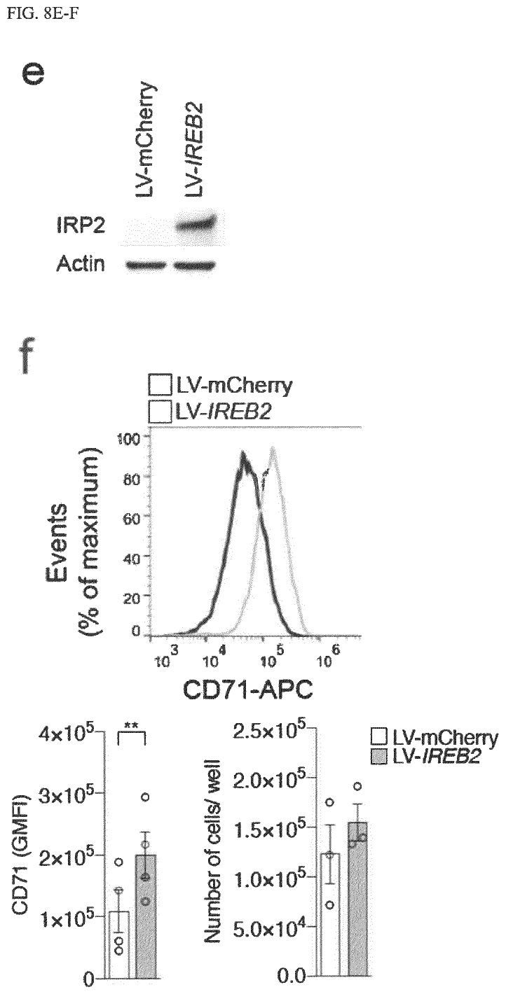

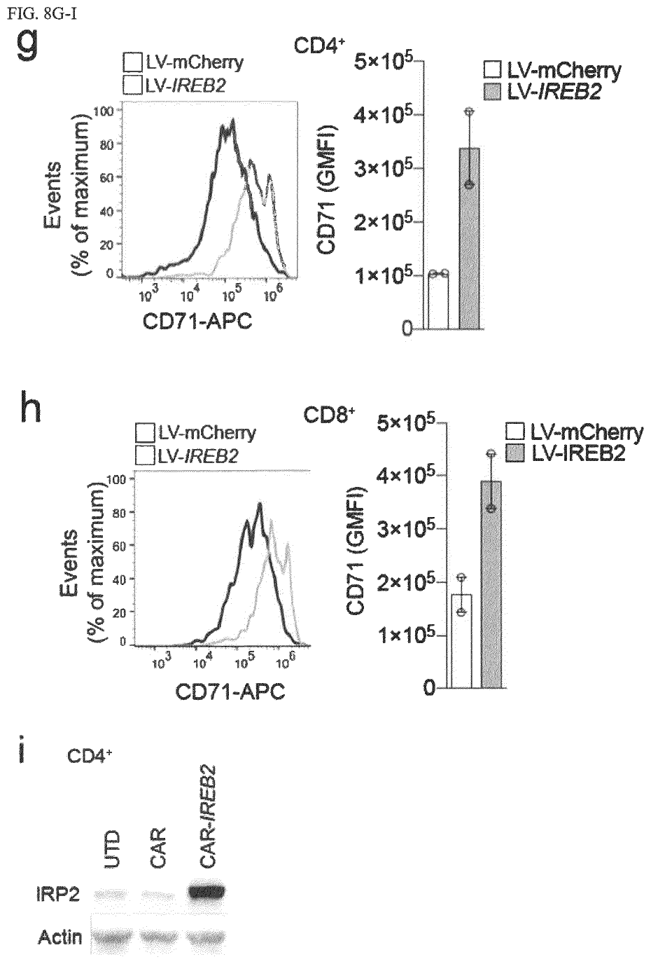

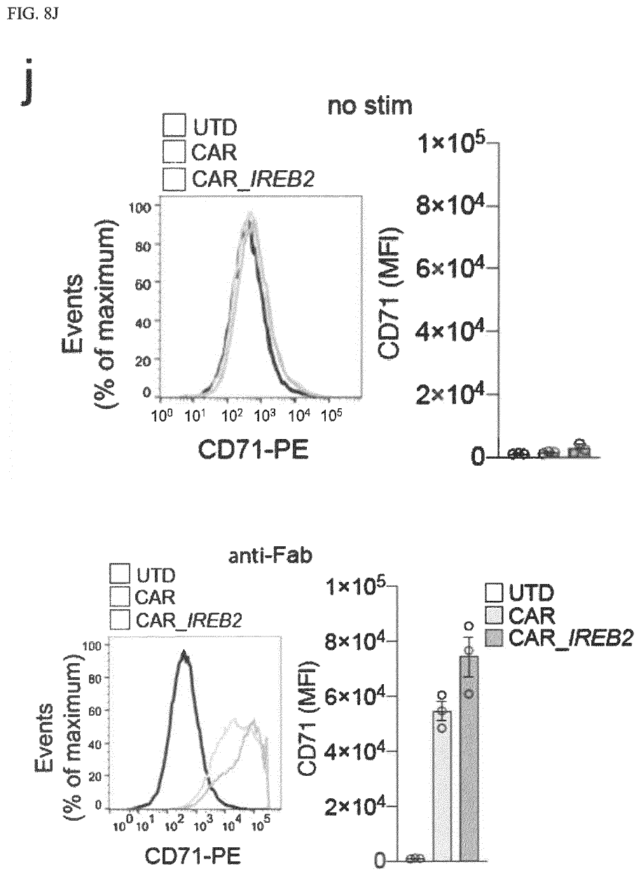

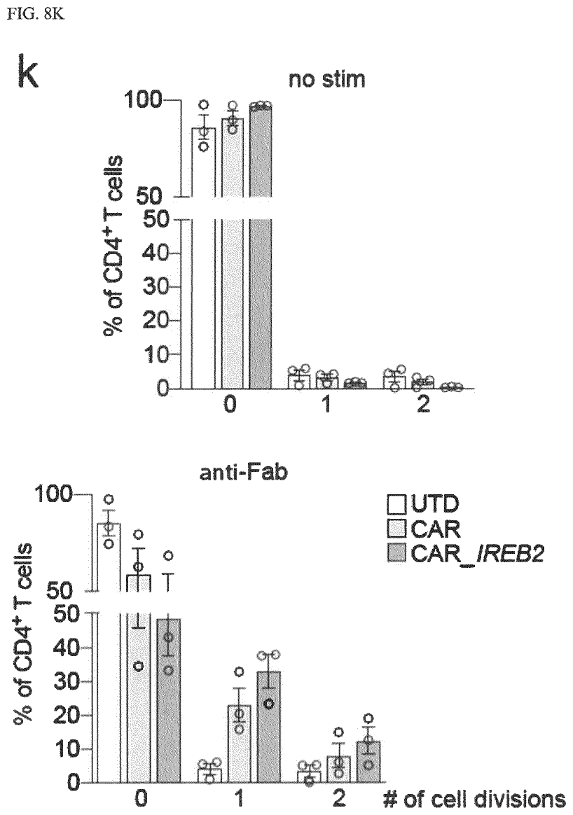

Enhanced recall responses of cytokine-enhanced (CE) NK cells reflect a promising feature for immune cell therapy against cancer. If and how CE NK cell metabolism underpins cytokine production, target cell clearance and proliferation remains unknown. To elucidate these key features of CE NK cells, the inventors used an established in vitro CE NK cell model that allowed comparison of naive (NV) vs. CE NK cells. Briefly, the inventors primed freshly isolated human NK cells with IL-12 and IL-18 (IL-12/IL-18) for 16 h, followed by a rest period in low dose IL-15 (IL-15 LD) to support survival. After 7 days of rest, features of NV vs. CE NK cells upon stimulation were compared (FIG. 1A). In line with previous data, priming of NK cells with IL-12/IL-18 augmented their capacity to produce IFN-γ upon re-stimulation (FIG. 1B). Of note, NK cells were similarly activated upon stimulation, as indicated by CD69 expression (FIG. 1C). To explore how cellular metabolism relates to the function of NV vs. CE NK cells at the transcriptional level, RNA sequencing (RNA-seq) was performed using unstimulated and cytokine-stimulated cells. Both unstimulated as well as activated NV and CE NK cells clustered separately in the principal component analysis (PCA). Activation was, however, a much stronger overall discriminating factor, indicating a relative similarity between the transcriptomes of NV and CE NK cells (FIG. 1D).

Rapid upregulation of aerobic glycolysis is a metabolic hallmark of activated lymphocytes, including NK cells. Unexpectedly, both NV and CE NK cells similarly upregulated gene transcripts encoding for glycolytic enzymes upon stimulation, with the exception of HK2 which was higher in CE than in NV NK cells (FIG. 1E). In line with the transcriptome data, metabolic flux assays showed increased basal and maximal glycolytic rates of activated compared to unstimulated cells, yet no difference between NV and CE NK cells was observed (FIG. 1F).

Likewise, uptake of the glucose analogue 2-NBDG was not different between unstimulated and activated NV and CE NK cells (FIG. 1G). To assess whether increased glycolytic metabolism was linked to the capacity of NV and CE NK cells to produce IFN-γ, the inventors stimulated NV and CE NK cells with IL-12/IL-18 in the presence of the hexokinase inhibitor 2-deoxy-d-glucose (2-DG). Inhibition of glycolysis during cytokine stimulation similarly reduced IFNG mRNA abundance and IFN-γ secretion in NV and CE NK cells (FIGS. 1H and 1I, left panel). Likewise, culturing NK cells in low glucose reduced production of IFN-γ in both subsets (FIG. 1I, upper panel). Together these data identified a similar increase in basal and maximal glycolytic activity upon activation of NV and CE NK cells, which was in both subsets required for efficient production of the key inflammatory cytokine IFN-γ.

### Example 2: Activated CE NK Cells are Characterized by High Levels of Cell-Surface CD71 and Rapid Cell Proliferation

To further characterize the metabolic profile of NV vs. CE NK cells, the inventors analyzed surface expression of the nutrient transporters CD98 and CD71 reported to be upregulated on activated NK cells. Upon stimulation, a slight and comparable increase in CD98 expression on both NK cell subsets was observed (FIG. 2A). In contrast, upregulation of the transferrin receptor CD71 was much greater on CE vs. NV NK cells, both when expressed as GMFI and percentage of positive cells (FIG. 2B). Increased cell surface expression of CD71 was reflected by an overall greater cellular abundance of CD71 protein as assessed by immunoblot analysis of whole cell lysates (FIG. 2C). To test whether differential cell surface expression of CD71 could also be driven by NK cell stimulation via activating receptors, both subsets were stimulated with HLA-deficient target cells (K562 cell line). Similar to cytokine stimulation, upregulation of CD71 was more prominent on K562-exposed CE than NV NK cells (FIG. 2D). To assess the functional capacity of increased CD71 expression, the inventors used fluorescently labeled transferrin to monitor transferrin uptake in NV and CE NK cells. These experiments revealed increased transferrin uptake in activated CE as compared to NV NK cells (FIG. 2E).

Expression of CD71 and rates of proliferation have previously been linked in neoplastic cells. To test whether this association also applied to NK cells, proliferation of NV and CE NK cells was monitored using CFSE dilution assays. Under both steady-state conditions and upon stimulation, CE NK cells proliferated to a greater extent than their NV counterparts (FIG. 2F). In line with differential proliferation rates, stimulated CE NK cells clustered when testing abundance of transcripts encoding for cell-cycle progression genes (FIG. 2G). To elucidate if increased transferrin uptake was linked to increased cell proliferation, the inventors used the intracellular iron chelator 2,2′-bipyridyl (BIP). These experiments revealed that BIP inhibited NK cell proliferation in a dose-dependent manner in both NV and CE NK cells (FIG. 2H). Of note, BIP had minimal effects on cell viability (data not shown), and no effect on NK cell activation as assessed by CD69 expression (FIG. 2I).

The pentose phosphate pathway (PPP), providing ribose 5-phosphate and NADPH for nucleotide synthesis and reducing equivalents, respectively, supports cell proliferation. In line with the increased proliferation observed in CE NK cells, cytokine stimulation increased mRNA abundance of several PPP-related genes more prominently in CE than NV NK cells (FIG. 2J, upper panel). The PPP inhibitor 6-aminonicotinamide (6AN) prevented expansion of cytokine-stimulated NK cells, further supporting the relevance of the PPP in promoting proliferation of both NV and CE NK cells (FIG. 2J, lower panel). Together, these experiments identified (i) preferential upregulation of CD71 on activated CE vs. NV NK cells, and (ii) increased proliferation of activated CE over NV NK cells, which relied on PPP activity.

### Example 3: CD71-Mediated Iron Uptake and Dietary Iron Availability Impact NK Cell Function

Recently, a mutation in the TFRC gene (TFRCY20H/Y20H) has been shown to impair B and T cell function, causing a primary immunodeficiency (PID). The mutation affects receptor-mediated endocytosis and compromises CD71-mediated iron uptake both in human cells and when introduced into mice. NK cell numbers in patients harboring this mutation are normal, however, functional properties have not been previously assessed. To test whether CD71 function and NK cell proliferation are linked, the inventors assessed CFSE dilution in IL-1S LD and IL-12/IL-18-stimulated wild type (WT) and TfrcY20H/Y20H murine NK cells, ex vivo. These experiments revealed a striking lack of IL-15 LD and IL-12/IL-18-induced proliferation among NK cells harboring the Tfrc mutation (FIG. 3A).

Given this strong phenotype, the inventors wondered whether mild iron deficiency might be sufficient to cause NK cell dysfunction. To explore this notion, the inventors first established systemic iron deficiency in a mouse model (FIG. 3B, upper panel). As expected, mice maintained on an iron-deficient diet for 6 weeks displayed reduced iron, ferritin and hematocrit levels in peripheral blood, while the unsaturated iron-binding capacity (UIBC) and the total iron-binding capacity (TIBC) increased as compared to mice kept on control diet (FIG. 3B, lower panel). While splenic T and B cell numbers were normal in mice with iron deficiency, NK cell numbers tended to be lower, possibly indicating selective sensitivity of these cells to systemic iron abundance (FIG. 3C). No impact of iron deficiency on the NK cell maturation phenotype was observed (FIG. 3D). However, upon MCMV infection, splenic NK cell-mediated viral control and IFN-γ production by NK cells tended to be reduced in mice maintained on iron-deficient diet, indicating impaired NK cell function (FIG. 3E. left panel. and 8F). Noteworthy, replication of Am157 MCMV evading NK cell-mediated control was unaffected by reduced iron levels (FIG. 3E, right panel). Together, these data established that CD71-mediated iron uptake had an important role in regulating NK cell proliferation. Further, reduced systemic iron levels significantly impaired immune control of MCMV infection in vivo, possibly by reducing NK-cell function. It remains to be elucidated, whether impaired NK cell-mediated immunity resulted from NK cell intrinsic or extrinsic factors.

### Example 4: CD71 Supports NK Cell Proliferation and Optimal Effector Function During Viral Infection

In order to test the functional importance of CD71-mediated iron uptake for NK cells, the inventors generated mice specifically lacking CD71 in NK cells, by crossing Ncr1Cre mice with Tfrcfl/fl mice (Tfrcfl/flNcr1Cre). Under homeostatic conditions, percentage and absolute numbers of NK cells in Tfrcfl/flNcr1Cre mice were slightly reduced in both liver and spleen as compared to Tfrcfl/f littermate controls (FIG. 4A). Percentage and absolute numbers of CD8+ and CD4+ T cells, as well as CD19+ B cells, were unaffected by NK cell specific deletion of CD71 (FIGS. 4B and 4C). Further, expression of terminal NK cell maturation markers was comparable between Tfrcfl/flNcr1Cre and Tfrcfl/fl mice (CD27, CD11b, KLRG1, CD62L and Ly6C) (FIGS. 4D and 4E). Likewise, the NK cell activating receptor, Ly49H, which is important for controlling MCMV infection, was equally expressed on Tfrcfl/flNcr1Cre and Tfrcfl/fl NK cells (FIG. 4F).

NK cell activation by MCMV drives proliferation of Ly49H+NK cells. To examine whether deletion of CD71 affects antigen-specific NK cell expansion in vivo, the inventors co-transferred congenic Ly49H+WT and Tfrcfl/flNcr1Cre NK cells into Ly49H-deficient (Klra8−/−) recipients (FIG. 4G, upper panel). The inventors then infected recipient mice with MCMV and tracked expansion of transferred NK cells. WT NK cells robustly expanded in liver, spleen, lung and blood, constituting 80-90% of the Ly49H+NK cell pool at 7 and 30 days post-infection (dpi) when compared to Tfrcfl/flNcr1Cre NK cells (FIG. 4G, lower panel). The inventors next addressed whether expansion of NK cells in lymphopenic hosts, which is driven by the availability of common-g-chain-dependent cytokines, was also dependent on CD71. To this end, the inventors transferred WT and Tfrcfl/flNcr1Cre NK cells at equal ratios into Rag2−/−IL2rg−/− recipient mice (FIG. 411, left panel). Similar to the infection experiment, at 6 dpi frequencies of Tfrcfl/flNcr1Cre NK cells were much lower than those of WT cells (FIG. 4H, right panel). Reduced numbers of Tfrcfl/flNcr1Cre NK cells in both adoptive transfer experiments could have resulted from either a lack of expansion, increased cell death or a combination of both. To assess how deletion of CD71 in NK cells related to their proliferation in vivo, WT and 7frcfl/flNcr1Cre NK cells were labeled with CFSE and transferred into recipient mice at equal ratios (FIG. 4I, upper panel). Recipients were then infected with MCMV and donor cells harvested at 3.5 dpi. CFSE dilution, and hence proliferation, of adoptively transferred Tfrcfl/flNcr1Cre NK cells in liver and spleen was significantly lower than that of WT cells (FIG. 4I, lower panel). These findings were further confirmed in in vitro proliferation studies, in which IL-15 LD and IL-12/IL-18-stimulated Tfrcfl/flNcr1Cre NK cells showed reduced proliferation compared to control cells (FIG. 4J).

To address whether deletion of CD71 affects NK cell-mediated viral control, the inventors challenged Tfrcfl/fl and Tfrcfl/flNcr1Cre mice with MCMV (FIG. 4K, upper panel). In line with the competitive transfer assays described above, a significant reduction in both percentage and absolute numbers of NK cells at 3.5 and 5.5 dpi was observed in both liver and spleen of Tfrcfl/flNcr1Cre mice (FIG. 4K, middle and lower panel). Insufficient expansion of NK cells was associated with higher splenic viral titers among Tfrcfl/flNcr1Cre mice at 3.5 dpi, with a similar trend observed in the liver (FIG. 4L). In addition, IFN-γ production of splenic and liver infiltrating Tfrcfl/flNcr1Cre NK cells was reduced upon MCMV infection (FIG. 4M). Of note, despite poor expansion and reduced effector capacity, CD71 deficiency did not impair terminal maturation of MCMV-challenged CD71 deficient NK cells, as indicated by CD27, CD11b and KLRG1 expression (FIG. 4N). Altogether, these data indicated a critical role of CD71 in NK cell proliferation both during infection and in a lymphopenic environment.

### Example 5: Glycolysis is Required for Induction of CD71 in Activated NK Cells

Our experiments established (i) iron uptake via CD71 as a critical metabolic checkpoint controlling NK cell proliferation; and (ii) highly preferential upregulation of CD71 on activated CE vs. NV NK cells. These findings prompted the inventors to ask how CD71 per se was regulated in NV and CE NK cells. To address this question, the inventors first assessed whether induction of CD71 relied on NK cell transcriptional activity. As in previous experiments, CD71 was induced to a greater extent in cytokine-stimulated CE than NV NK cells (FIG. 5A). In both subsets inhibition of transcription (using Actinomcyin D) entirely prevented stimulation-induced upregulation of CD71, as did blocking of translation (using cycloheximide) (FIG. 5A). Thus, transcription and translation were similarly required in both cell subsets. Glycolytic reprogramming has previously been demonstrated to drive transcription in activated NK cells (72). As glycolysis was similarly triggered in activated NV and CE NK cells (FIG. 1F), the inventors examined the possibility that glycolytic metabolism may differentially impact TFRC (which encodes CD71) transcription between NV and CE NK cells. TFRC mRNA abundance was indeed higher in activated CE than NV NK cells, yet similarly reduced when inhibiting glycolysis with 2-DG (FIG. 5B). Cell surface expression of CD71 and total CD71 levels followed the same pattern when exposing cells to 2-DG (FIG. 5C). Dependence on glucose to induce CD71 expression was recapitulated upon activation of NK cells in low glucose medium and translated into reduced transferrin uptake in 2-DG treated NK cells (FIGS. 5D and 5E). Glycolysis thus enabled transcription and translation of CD71. However, no evidence was found that glycolysis regulated the differential abundance of CD71 in activated CE vs. NV NK cells.

c-Myc has been established as a key regulator of TFRC transcription in various immune cells. Given the increased abundance of TFRC mRNA among activated CE over NV NK cells (FIG. 5B), preferential c-Myc induction in CE NK cells could explain differential regulation of CD71 between activated CE and NV NK cells. Yet, c-Myc was robustly but equally induced in both NK cell subsets (FIG. 5F). Together these data established a symmetric need in activated NV and CE NK cells for (i) continuous transcription and translation to support expression of CD71 and (ii) glycolytic reprogramming as a metabolic requirement for CD71 expression.

### Example 6: Cytokine Priming Induces the IRP/IRE Regulatory System

Many genes involved in cellular iron homeostasis contain iron responsive elements (IREs) in the 5′ or 3′UTR of their mRNA. Iron regulatory proteins 1 and 2 (IRP1 and IRP2) bind IREs, thereby controlling mRNA stability and translation. TFRC mRNA contains 5 IREs in the 3′UTR; binding of IRPs stabilizes the mRNA and facilitates translation. As described, this would occur under iron-deficient conditions. Hence, the inventors hypothesized that increased abundance of IRPs, selectively in CE NK cells, could be a possible mechanism regulating enhanced CD71 expression in activated CE NK cells. At the mRNA level, abundance of both IRP transcripts, ACO1 and IREB2, was similar in NV and CE NK cells (FIG. 6A, upper panel). Protein abundance of IRP1 and IRP2 was, however, higher in quiescent and activated CE NK cells (FIG. 6A, lower panel). This finding was compatible with a novel role for IRPs, generating a pseudo iron deficient state in a cell subset-specific manner, thereby post-transcriptionally controlling abundance of a distinct set of proteins.

To extend this observation, the inventors analyzed transcript abundance of known IRE containing mRNAs expressed in NK cells (FIG. 6B). The inventors included the critical eukaryotic translation initiation factor 4E (eIF4E) in the list of IRE containing mRNAs, since searching for ironresponsive elements (SIRE) algorithm revealed an IRE-like motif in the 3′UTR of the EIF4E mRNA (data not shown). This analysis prompted the inventors to assess the transcriptional and translational pattern for EIF4E. The pattern observed for CD71 was somewhat recapitulated, activated CE NK cells expressed more EIF4E transcript and clearly more eIF4E protein (FIG. 6C). Yet, already quiescent CE NK cells expressed increased levels of eIF4E compared to NV NK cells. Of note, despite higher abundance of eIF4E, global protein translation was not discernibly different between activated NV and CE NK cells, as assessed by using L-homopropargylglycine (HPG) incorporation assays (FIG. 6D). In addition, the inventors noted increased Fr1l mRNA abundance in activated CE NK cells (FIG. 6B). FTH1 mRNA contains an IRE in the 5′UTR and binding of IRPs to 5′UTRs IREs inhibits translation. This constellation enabled the inventors to test the hypothesis of a pseudo iron deficiency driven by selective increase in IRPs abundance in CE NK cells. Indeed, despite higher transcript levels, protein abundance of ferritin heavy chain 1 (the gene product of FTH1) was, if anything, lower in both unstimulated and activated CE NK cells (FIG. 6E). This finding was highly suggestive of IRPs, with their CE and NV NK cell specific abundance, being involved in regulating FTH1 mRNA translation. Together these data established a regulatory axis, selectively induced in CE NK cells, in which pseudo iron deficiency enables increased translation of CD71—and hence proliferation—of activated CE NK cells.

### Example 7: Expression of IRPs in CAR T Cells

### CAR T Cell Generation

Sequences of the antigen binding domain, the transmembrane domain, the CD3ζ domain, and the CD28 costimulatory domain of the CAR and IRP1 and/or IRP2 are cloned into a corresponding lentiviral vector. If necessary, the IRPs and the CAR sequences are cloned into separate lentiviral packaging vectors. Lentiviral production is performed in a suitable cell line. CD8+ and/or CD4+ T cells are isolated from peripheral blood mononuclear cells from the patient or a healthy donor and activated with anti-CD3 and anti-CD28 (soluble or bead-bound) in the presence of IL-2. One to two days post-activation, soluble antibodies or beads are removed and cells are transduced with the lentivirus for approximately 18 hours and media is replaced with fresh media supplemented with IL-2. CAR and IRP expression will be confirmed by flow cytometry at indicated time points.

### Optional:

CD8+ and/or CD4+ T cells are isolated from peripheral blood mononuclear cells from the patient or a healthy donor and activated with anti-CD3 and anti-CD28 (soluble or bead-bound) in the presence of IL-2. One day post-activation cells are transduced with the lentivirus for approximately 48 hours and media is replaced with fresh media supplemented with IL-2. Five days post activation beads are removed and replaced with media containing IL-15 and IL-7. CAR and IRP expression will be confirmed by flow cytometry at indicated time points.

### Proliferation of CAR T Cells In Vitro

To analyze cell proliferation of CAR T cells, cells are loaded prior to activation with the cell-proliferation dye carboxyfluorescein succinimidyl ester (CFSE, 1 sM, Molecular probes, USA) and seeded in 96-well plates. A fixable live-dead cell stain (Fixable Viability Dye, eBioscience or Zombie Aqua, Biolegend) is used to exclude dead cells prior to sample acquisition. CFSE dilution is analyzed at various time points post-stimulation by flow cytometry.

### Proliferation of CAR T Cells In Vivo

To analyze cell proliferation of CAR T cells in vivo, CAR T cells overexpressing at least one IRP and CAR T cells not overexpressing any IRP are adoptively transferred into a corresponding murine tumor model and the frequency and number of transferred cells is analyzed at various time points. In certain experiments, CAR T cells overexpressing at least one IRP and CAR T cells not overexpressing any IRP are loaded with cell proliferation dye CFSE prior to transfer to analyze the proliferation in vivo.

### Mouse Tumor Models

CAR T cells overexpressing at least one IRP and CAR T cells not overexpressing any IRP are adoptively transferred into a corresponding murine tumor model. Depending on the tumor model, in certain experiments the tumor diameter is measured. Depending on the tumor model, in certain experiments lungs are dissected and fixed in the corresponding buffer and numbers of nodules are counted using a microscope. Depending on the tumor model, in certain experiments the survival rate is analyzed.

### Example 8: Materials and Methods

### Mice

Animal experiments performed at the University of Rijeka, Faculty of Medicine, were approved by Ethical Committee of the Faculty of Medicine, University of Rijeka, and Ethical Committee at the Croatian Ministry of Agriculture, Veterinary and Food Safety Directorate (UP/1-322-01/18-01/44). Mice were strictly age- and sex-matched within experiments and were held in SPF conditions. Animal handling was in accordance with the guidelines contained in the International Guiding Principles for Biomedical Research Involving Animals.

Wild-type C57BL/6J (B6, strain 000664), B6 Ly5.1 (strain 002014), Tfrcfl/fl (strain 028363) and Rag2−/−yc−/− (strain 014593) mice were purchased from the Jackson laboratory. Ncr1Cre mice were kindly provided by V. Sexl (Vienna, Austria) and B6.Ly49h−/− were kindly provided by Silvia M. Vidal (Montreal, Canada). In some experiments mice were put on iron-deficient diet and corresponding control diet for 6 weeks (C1038 and C1000, Altromin).

Animal experiments at the University of Basel were performed in accordance with local rules for the care and use of laboratory animals. Mice were strictly age- and sex-matched within experiments and were held in SPF conditions. Wild-type C57BL/6J (B6, strain 000664) mice were purchased from Jackson Laboratories (USA) and TfrcY20H/Y20H mice were kindly provided by R. Geha (Boston, USA).

### Hematologic Analyses

The serum iron, ferritin, unsaturated iron binding capacity (UIBC) and total iron binding capacity (TIBC) were determined using AU5800 Analyzer (Beckman Coulter). Hematocrit was determined using hematology analyzer DxH500 (Beckman Coulter). Measurements were conducted at the Clinical Institute of Laboratory Diagnostics (Clinical Hospital Center, Rijeka, Croatia).

### Viruses

The bacterial artificial chromosome-derived murine cytomegalovirus (BAC-MCMV) strain pSM3fr-MCK-2fl clones 3.3 has previously been shown to be biologically equivalent to MCMV Smith strain (VR-1399; ATCC) and is herein after referred to as wild-type (WT) MCMV231. pSM3fr-MCK-2fl clone3.3 and Am157 were propagated on mouse embryonic fibroblasts (MEFs)232. Animals were infected intravenously (i.v.) with 2×10⁵ plaque forming units (PFU). Viral titers were determined on MEFs by standard plaque assay.

### Adoptive Transfer Experiments

Adoptive co-transfer studies were performed by transferring splenocytes from WT B6 (CD45.1) and Tfrcfl/flNcr1Cre (CD45.2) mice in an equal ratio into B6Ly49h−/− and, respectively, into Rag2−/−yc−/− recipients 1 day prior to MCMV infection. For cell proliferation assays in vivo, splenocytes were loaded, prior to transfer, with cell proliferation dye carboxyfluorescein succinimidyl ester (5 μM CFSE, Molecular probes, USA).

### Human NK Cell Isolation and Cell Culture

Blood samples were obtained from healthy donors after written informed consent. Peripheral blood mononuclear cells were isolated by standard density-gradient centrifugation protocols (Lymphoprep; Fresenius Kabi). NK cells were negatively selected using EasySep negative NK cell isolation kit (Stemcell). Human NK cells were maintained in RPMI-1640 medium (Invitrogen) supplemented with 10% heat-inactivated human AB serum, 50 U/ml penicillin (Invitrogen) and 50 μg/ml streptomycin (Invitrogen) (R10AB). To generate CE NK cells, isolated NK cells were primed in R10AB containing IL-12 (10 ng/ml, R&D systems), IL-15 (1 ng/ml, PeproTech) and IL-18 (50 ng/ml, R&D systems) over-night. The next day cells were washed twice with PBS and maintained in R10AB containing IL-15 (1 ng/ml) until stimulation. Every 2-3 days 50% of the medium was replaced with fresh IL-IS (1 ng/ml). After 7 days cells were stimulated in R10AB containing IL-12 (10 ng/ml), IL-15 (1 ng/ml) and IL-18 (50 ng/ml) or with K562 leukemia targets (effector: target ratio, 5:1) for 6 hours. When indicated, cells were pre-incubated with 2-deoxy-D-glucose (10 mM, Sigma-Aldrich), Actinomycin D (1 and 10 μM, Sigma-Aldrich), Cycloheximide (10 and 100 μg/ml, Sigma-Aldrich), 2,2′-Bipyridyl (1, 10, 50 and 100 μM, Sigma-Aldrich) or 6-aminonicotinamide (50 μM, Sigma-Aldrich) for 30 min and then stimulated in R10AB containing IL-12 (10 ng/ml), IL-1S (1 ng/ml) and IL-18 (50 ng/ml) for 6 hours.

The NK cell lines, NK92 and NKL were maintained in R10AB supplemented with IL-2 (50 U/ml). Jurkat and K562 cell lines were maintained in RPMI-1640 medium (Invitrogen) supplemented with 10% heat-inactivated human fetal bovine serum (FBS), 50 U/ml penicillin (Invitrogen) and 50 μg/ml streptomycin (Invitrogen) (R10FBS). 293T human embryonic kidney (HEK-293T) cells were maintained in DMEM medium (Invitrogen) supplemented with 10% heat-inactivated human fetal bovine serum (FBS), 50 U/ml penicillin (Invitrogen) and 50 μg/ml streptomycin (Invitrogen).

### Flow Cytometry Analysis of Human Cells

For surface staining NK cells were stained for 30 min at 4° C. with saturating concentrations of antibodies. Following antibodies were used: anti-human CD71 (clone CY1G4, Biolegend), antihuman CD69 (clone FN50, Immunotools), anti-human CD98 (clone MEM-108, Biolegend). Samples were acquired using a BD AccuriC6 or a CytoFLEX flow cytometer (Beckman Coulter). Data were analyzed with Flowjo®_V10.5 (Tree Star, USA).

For cell proliferation assays, NK cells were loaded prior to activation with the cell-proliferation dye CFSE (1 μM, Molecular probes, USA) and seeded in 96-well plates. Cells were washed twice and maintained in R10AB with IL-15 (1 ng/ml), when indicated in the presence of inhibitors. A fixable live-dead cell stain (Fixable Viability Dye, eBioscience or Zombie Aqua, Biolegend) was used to exclude dead cells prior to sample acquisition. CFSE dilution was analyzed 65 hours post-stimulation by flow cytometry. Samples were acquired using a BD AccuriC6 or a CytoFLEX flow cytometer (Beckman Coulter). Data were analyzed with Flowjo®_V 10.5 (Tree Star, USA).

### Flow Cytometry Analysis of Murine Cells

Lymphocytes from spleens were isolated by meshing organs and filtering them through a 100-100 μm strainer. To isolate lymphocytes from liver, the tissue was meshed and filtered through a 100 μm strainer and purified using a discontinuous gradient of 40% over 80% Percoll. Red blood cells in spleen and liver were lysed using erythrocyte lysis buffer. Cells were pretreated with Fc block (clone 2.4G2) and a fixable live-dead cell stain (Fixable Viability Dye, eBioscience) was used to exclude dead cells. Cells were stained for 30 min at 4° C. with saturating concentrations of antibodies. Following antibodies purchased from Thermo Fisher Scientific were used: anti-mouse CD8α (clone 53-6.7), anti-mouse CD45.2 (clone 104), anti-mouse CD4 (clone RM4-5), anti-mouse CD69 (clone H1.2F3), anti-mouse CD45.1 (clone A20), anti-mouse CD3e (clone 145-2C11), anti-mouse CD19 (clone 1D3), anti-mouse NK1.1 (clone PK136), antimouse NKp46 (clone 29A1.4), anti-mouse CD62L (clone MEL-14), anti-mouse Ly6c (clone HK1.4), anti-mouse KLRG1 (clone 2F1), anti-mouse Ly49H (clone 3D10), anti-mouse CD11b (clone M1/70) and anti-mouse CD27 (clone 0323). Samples were acquired using a BD FACSAria. Data were analyzed with Flowjo®_V10.5 (Tree Star, USA).

For intracellular cytokine staining upon MCMV infection, lymphocytes from spleen and liver of MCMV-infected mice were isolated as indicated above. Cells were resuspended in RPMI-1640 medium supplement with 10% fetal bovine serum (Thermo Fisher Scientific), 50 U/ml penicillin (Invitrogen), 50 μg/ml streptomycin (invitrogen) and 50 μM 2-mercaptoethanol (Thermo Fisher Scientific) (R10FBS) in the presence of IL-2 (500 IU/ml). Cells were incubated at 37° C. in the presence of brefeldin A (eBioscience) for 5 hours. Cells were surface-stained, followed by fixation and permeabilization according to the manufacturer's protocol (BD Biosciences). Intracellular cytokines were stained using mouse-anti IFN-γ (clones XMG1.2, Thermo Fisher Scientific). Samples were acquired using a BD FACSAria. Data were analyzed with Flowjo®_V10.5 (Tree Star, USA).

For cell proliferation assays, lymphocytes were loaded prior to activation with the cellproliferation dye CFSE (1 μM, Molecular probes, USA) and seeded in U-bottom 96-well plates (5×10⁵ cells/well). Cells were stimulated in R10FBS containing IL-12 (10 ng/ml, PeproTech), IL-15 (10 ng/ml, PeproTech) and IL-18 (50 ng/ml, R&D Systems) for 16 hours. Cells were washed twice and maintained in R10FBS containing IL-15 (10 ng/ml). CFSE dilution was analyzed 65 hours post-stimulation by flow cytometry. A fixable live-dead cell stain (Fixable Viability Dye, eBioscience or Zombie Aqua, Biolegend) was used to exclude dead cells. Samples were acquired using a BD FACSAria or CytoFLEX flow cytometer (Beckman Coulter). Data were analyzed with Flowjo®_V10.5 (Tree Star, USA).

### Seahorse Metabolic Flux Analyzer

A Seahorse XF-96e extracellular flux analyzer (Seahorse Bioscience, Agilent) was used to determine the metabolic profile of cells. NK cells were plated (3×10⁵ cells/well) onto Celltak (Corning, USA) coated cell plates. Mitochondrial perturbation experiments were carried out by sequential addition of oligomycin (1 μM, Sigma), FCCP (2 μM, Carbonyl cyanide 4-(trifluoromethoxy) phenylhydrazone, Sigma), and rotenone (1 μM, Sigma). Oxygen consumption rates (OCR, pmol/min) and extracellular acidification rates (ECAR, mpH/min) were monitored in real time after injection of each compound.

### 2-NBDG Uptake

NK cells were seeded in U-bottom 96-well plates (2×10⁵ cells/well). When indicated cells were pre-incubated for 30 min with inhibitors and stimulated in R10AB containing IL-12 (10 ng/ml), IL-15 (1 ng/ml) and IL-18 (50 ng/ml) for 6 hours. Cells were then incubated in medium containing 20 μM 2-NBDG (Invitrogen) for 15 min and analyzed by flow cytometry. Samples were acquired using a BD AccuriC6 flow cytometer. Data were analyzed with Flowjo®_V10.5 (Tree Star, USA).

### IFN-γ Measurement in Human NK Cells

NK cells were seeded in U-bottom 96 well plates (2×10⁵ cells/well) using R10AB. When indicated cells were pre-incubated for 30 min with inhibitors and stimulated in R10AB containing IL-12 (10 ng/ml), IL-15 (1 ng/ml) and IL-18 (50 ng/ml) for 6 hours. Cell supernatants were harvested after stimulation and IFN-γ was measured using a human Th1 cytokine bead-based immunoassay (Legendplex, Biolegend) according to manufacturer's protocol.

### Transferrin Uptake Assay

NK cells were seeded in U-bottom 96-well plates (2×10⁵ cells/well). When indicated cells were pre-incubated for 30 min with inhibitors and stimulated in R10AB containing IL-12 (10 ng/ml), IL-15 (1 ng/ml) and IL-18 (50 ng/ml) for 4 hours. Cells were then stimulated in RPMI-1640 medium containing 5% BSA and IL-12 (10 ng/ml), IL-15 (1 ng/ml) and IL-18 (50 ng/ml) for 2 hours. After stimulation cells were washed with RPMI-1640 containing 0.5% BSA before incubation with transferrin-alexa488 conjugate (Tf-488, 10 μg/ml, Thermo Fisher Scientific) for 15 min. Transferrin uptake was stopped by washing cells in ice-cold acidic buffer (150 mM NaCl, 20 mM citric acid and pH: 5). Cells were resuspended in FACS buffer and analyzed by flow cytometry. Samples were acquired using a BD AccuriC6 flow cytometer. Data were analyzed with Flowjo®_V10.5 (Tree Star, USA).

### HPG Incorporation Assay

NK cells were seeded in 96-well plates (2×10⁵ cells/well). Cells were stimulated in R10AB containing IL-12 (10 ng/ml), IL-15 (1 ng/ml) and IL-18 (50 ng/ml) for 4.5h and afterwards incubated for 1.5h in methionine-free RPMI-1640 medium containing 10% dialyzed FBS and IL-12 (10 ng/ml), IL-15 (1 ng/ml) and IL-18 (50 ng/ml). Click-IT®HPG (50 μM, Life Technologies) was added for the last 30 min of the incubation. HPG incorporation into NK cells was stained with Click-iT® reaction cocktail (Thermo Fisher Scientific) and detected by flow cytometry. Samples were acquired using a BD AccuriC6 flow. Data were analyzed with Flowjo®_V10.5 (Tree Star, USA).

### Immunoblot Analysis

Protein concentrations were determined by BCA protein assay kit (Thermo Fisher Scientific). Total cell lysates were separated using 4%-15% Mini Protean TGX Gel (Bio-Rad, Hercules Calif., USA), and transferred to nitrocellulose membranes using Trans-Blot Turbo Transfer (Bio-Rad, Hercules Calif., USA). Membranes were probed with the following antibodies: anti-human CD71 mAb (13113), anti-human IRP1 mAb (20272), anti-human IRP2 mAb (37135), anti-human FTH1 mAb (4393), anti-human eIF4E mAb (2067), anti-human c-Myc mAb (5605) and anti-human β-actin mAb (3700) (all from Cell Signaling, USA). Blots were stained with appropriate secondary antibodies and the odyssey imaging system (LICOR, Lincoln Nebr., USA) was used for visualization, and the ImageJ software (1.48v) for quantification.

### RNA Sequencing

RNA-seq was performed by Admera Health (USA). In brief, samples were isolated using ethanol precipitation. Quality check was performed using Tapestation RNA HS Assay (Agilent Technologies, USA) and quantified by Qubit RNA HS assay (Thermo Fisher Scientific). Ribosomal RNA depletion was performed with Ribo-zero Magnetic Gold Kit (MRZG12324, Illumina Inc., USA). Samples were randomly primed and fragmented based on manufacturer's recommendation (NEBNext® Ultra™ RNA Library Prep Kit for Illumina®).

First strand was synthesized using Protoscript II Reverse Transcriptase with a longer extension period (40 min for 42° C.). All remaining steps for library construction were used according to the NEBNext® Ultra™ RNA Library Prep Kit for Illumina®. Illumina 8-nt dual-indices were used. Samples were pooled and sequencing on a HiSeq with a read length configuration of 150 paired-end.

Reads were aligned to the human genome (UCSC version hg38AnalysisSet) with STAR (version 2.5.2⁴) using the multi-mapping settings ‘--outFilterMultimapNmax 10--outSAMmultNmax 1’. The output was sorted and indexed with samtools (version 1.7) and picard markDuplicates (version 2.9.2) was used to collapse samples run on different sequencing lanes. The qCount function of QuasR (version 1.20.05) was used to count the number of read (5′ends) overlapping with the exons of each gene assuming an exon union model (RefSeq genes downloaded from UCSC on 2017-09-01). All subsequent gene expression data analysis was done within the R software (R Foundation for Statistical Computing, Vienna, Austria). The differentially expressed genes were identified using the edgeR package (version 3.22.5).

### Quantitative Real-Time PCR

RNA was isolated from NK cells using Trizol (Thermo Fisher Scientific) and chloroform (Sigma-Aldrich) according to manufacturer's protocol, then purified with RNeasy RNA purification mini kit (QIAGEN, Germany). RNA concentration was determined using the NanoDrop 2000C (Thermo Fisher Scientific). From purified RNA, cDNA was synthesized using the reverse transcriptase kit GoScript™ Reverse Transcriptase (Promega). Quantitative PCR for IFNG, TFRC and 18S mRNA was done in triplicate using commercially designed primers from Life Technologies (Hs00989291_m1, Hs00951083_m1, Hs03003631_g1). PCR reactions were performed using Go Tag G2 DNA Polymerase (Promega) according to manufacturer's protocol.

### RNA-Mediated Interference

NK92, NKL or Jurkat cells (2×10⁶) were transfected with pools of siRNA targeting ACO1, IREB2 or control-scrambled siRNA (each 10 pmoles) (QIAGEN) using the AMAXA cell line V nucleofection kit (Lonza). Afterwards cells were rested for 72 hours and phenotypically and functionally analyzed. Knockdown efficiency was assessed by immunoblot analyses of the respective proteins.

### CRISPR Editing

A 24-well cell culture plate with 1 ml R10AB containing IL-2 (50 U/ml; NK92 cells) or R10FBS (Jurkat cells) was prepared and pre-warmed at 37° C. For CRISPR-Cas9 mediated IREB2 gene knockout following sgRNAs from IDT were used: Hs.Cas9.IREB2.1 AA (Ref no. 220257866) or Alt-R CRISPR-Cas9 Negative Control (Ref no. 224163224). Guide RNA complexes were formed by combining the crRNA and tracrRNA in equal molar amounts in IDT Duplex buffer (30 mM HEPES, pH 4.5, 100 mM potassium acetate) at 20 μM concentration by heating the oligos at 95° C. for 5 min and slowly cooling to room temperature. An equal volume of CAS9 nuclease (QB3 MacroLab, University of California, Berkeley) was added and incubated at room temperature for 15 min. NK92 or Jurkat cells (2 ×10⁶) were washed in PBS and resuspended in electroporation solution (AMAXA cell line V nucleofection kit, Lonza). RNP solution (3 μM final RNP concentration) was added and electroporated with the recommended program. Cells were transferred into pre-warmed media and rested cells for indicated time points. NK92 cells were rested for 5 days, followed by phenotypically and functionally analysis. Knockdown efficiency of IRP2 was assessed by immunoblot. Jurkat cells were rested for 2 days, followed by single cell sorting into 96-well plates. Clones were expanded and knockout efficiency of IRP2 was assessed by immunoblot analyses and positive clones were expanded for phenotypical and functional analysis and lentiviral transduction of IRP2.

### IRP2 CAR Construction

The human IRP2 was synthesized as gene strings (GeneArt, Thermo Fischer Scientific). IRP2 (NM_004136.4) was then cloned into a third-generation self-inactivating lentiviral expression vector, pELNS, with expression driven by the elongation factor-1α (EF-1α) promoter, in frame with T2A and the second generation anti-PSMA CAR. The scfv for the anti-PSMA CAR derived from monoclonal antibody J591 used as the tumor-targeting moiety, while the intracellular domain consists of CD28 costimulatory domain and CD3zeta chain. In the control vector IRP2 has been substituted with the reporter gene eGFP.

### Recombinant Lentivirus Production

24 hours before transfection, HEK-293 cells were seeded (5×10⁶ cells/5 ml media). All plasmid DNA was purified using the Endotoxin-free Plasmid Maxiprep Kit (Sigma). HEK-293T cells were transfected with 1.3 pmoles psPAX2 (lentiviral packaging plasmid) and 0.72 pmoles pMD2G (VSV-G envelope expressing plasmid) and 1.64 pmoles of pLV-EFIA>mCherry(ns):P2A:EGF or PLV-EIFIA>hIREB2:P2A:EGFP (Vector Builder) using Lipofectamine 2000 (Invitrogen) and Optimem medium (Invitrogen, Life Technologies). The viral supernatant was collected 48 and 72 hours after transduction. Viral particles were concentrated using VIVASPIN 20 (Sartorius) and viral supernatants were stored at −80° C. Lentiviral particles of CAR constructs were produced as described by Giordano-Attianese et al., Nat Biotechnol, 2020, 38, 426-432).

### Lentiviral Transduction of Jurkat Cells

Jurkat cells were seeded in U-bottom 96 well plates (5×10⁵ cells/well) using R10FBS. Viral supernatant was thawed and Jurkat cells were transduced with different virus dilutions ranging from 1:16 to 1:1′160′000. Plate was centrifuged at 400×g for 3 minutes and incubated for at 37° C. for 24 hours. Afterwards medium was changed and cells were rested for 2 more days. Transduction efficiency was assessed by analyzing GFP expression by flow cytometry. GFP⁺ cells were flow-sorted (frequency 10-30% positive cells) and expanded for phenotypical analysis. Lentiviral overexpression of IRP2 was assessed by immunoblot analyses.

### Lentiviral Transduction of Primary T Cells

Blood samples were obtained from healthy donors after written informed consent. Peripheral blood mononuclear cells (PBMCs) were isolated by standard density-gradient centrifugation protocols (Lymphoprep; Fresenius Kabi). CD4+ and CD8⁺ T cells were positively selected using magnetic CD4⁺ and CD8+ beads (Miltenyi Biotec). Purified CD4⁺ and CD8⁺ T cells were cultured in R10AB. CD4⁺ and CD8⁺ T cells were plated into a 24-well cell culture plate and stimulated with anti-CD3 and anti-CD28 monoclonal antibody-coated beads (Invitrogen, Life Technologies) in a ratio of 1:1 in R10AB containing IL-2 (150 U/ml). T cells were transduced with lentiviral particles at 18-22 hours after activation in cell culture plates coated with Retronectin (Takara Bio). Every 24 hours medium was replaced with fresh IL-2 (150 U/ml). 5 days after transduction cells were analyzed for CD71 expression by flow cytometry. Samples were acquired using CytoFLEX flow cytometer (Beckman Coulter). Data were analyzed with Flowjo® V10.5 (Tree Star, USA). Lentiviral transduction of primary T cells with CAR constructs were conducted as described⁷.

### Activation and Proliferation of Transduced Primary CAR T Cells

Transduced T cells expressing the CAR or CAR_IREB2 were adjusted for equivalent CAR expression. To stimulate the CAR a polyclonal anti-Fab antibody (Jackson Immuno Research) was used. Briefly, a 96-well plate was coated with 20 μg/ml anti-Fab in PBS for 4 hours at 37° C. Primary T cells were loaded prior to activation with the cell-proliferation dye Cell Trace violet (CTV; 1 μM, Thermo Fisher Scientific). Plate was washed twice and stained T cells were seeded (1×10⁵/well) and stimulated for 5 days. CTV dilution and CD71 expression was analyzed by flow cytometry. Samples were acquired using BD FACS LSR II flow cytometer (BD Bioscience). Data were analyzed with Flowjo®_V10.5 (Tree Star, USA).

### Statistical Analysis

Data are presented as mean±SEM. Statistical significance was determined by either using unpaired two-tailed Student's t test or paired two-tailed Student's t test using GraphPad Prism 8.00 (GraphPad Software). For comparison of increases (before versus after) in paired samples, a simple linear-regression model was used. P values of less than 0.05 were considered statistically significant.

### Example 9: The IRP/IRE Regulatory System Orchestrates CD71 Expression in NK Cells

Thus far, the experiments established (i) iron uptake via CD71 as a critical metabolic checkpoint controlling NK cell proliferation, and (ii) preferential upregulation of CD71 on activated CE as compared to NV NK cells. Next, the inventors asked how CD71 per se was regulated in NV and CE NK cells. To address this question, the inventors first assessed whether induction of CD71 relied on NK cell transcriptional activity. As in previous experiments, CD71 was induced to a greater extent in cytokine-stimulated CE than NV NK cells (FIG. 5A, upper panel). In both subsets, inhibition of transcription—using actinomycin—prevented stimulation-induced upregulation of CD71, as did blocking of translation with cycloheximide (FIG. 5A lower panel). Thus, transcription and translation were similarly required in both cell subsets. Next, we examined the possibility that transcription of TFRC might be differentially regulated between NV and CE NK cells. To do so, we analyzed transcript abundance of TFRC. Indeed, TFRC mRNA levels were higher in activated CE than NV NK cells, yet further induced in both subsets upon stimulation (FIG. 7A). c-Myc is a key transcription factor regulating TFRC in various immune cells. Given the increased abundance of TFRC mRNA among activated CE over NV NK cells, preferential c-Myc induction in CE NK cells could thus explain differential regulation of CD71 between activated CE and NV NK cells. However, c-Myc was equally induced in both NK cell subsets (FIG. 5F). Together these data established a symmetric need in activated NV and CE NK cells for continuous transcription and translation to support expression of CD71.

Many genes involved in cellular iron homeostasis contain iron responsive elements (IREs) in the 5′ or 3′ UTR of their mRNA. Iron regulatory proteins 1 and 2 (IRP1 and IRP2) bind IREs, thereby controlling mRNA stability and translation. TFRC mRNA contains 5 IREs in the 3′UTR, where binding of IRPs stabilizes the mRNA and facilitates translation. As per text book, this occurs under iron-deficient conditions. The inventors reasoned that, irrespective of cellular iron abundance, increased expression of IRPs selectively in CE NK cells would explain higher TFRC transcript abundance and enhanced activation-dependent CD71 expression in these cells. Supporting this idea, protein abundance of both IRP1 and IRP2 were higher in quiescent and activated CE NK cells (FIG. 6A middle and lower panels). These data were compatible with IRPs generating a pseudo iron deficient state in a cell subset-specific manner, namely in CE NK cells, thus selectively controlling abundance of a distinct set of proteins at the post-transcriptional level. To further probe this notion, we analyzed transcript and protein abundance of FTH1 (encoding the ferritin heavy chain), which contains an IRE in the 5′UTR—where binding of IRPs inhibits translation. FTH1 mRNA was increased in activated CE NK cells (FIG. 7B)—yet despite these higher transcript levels, protein abundance of ferritin heavy chain was, if anything, lower in both unstimulated and activated CE NK cells (FIG. 6E). This finding was highly suggestive of IRPs, with their CE vs. NV NK cell specific abundance, being involved in regulating translation of IRE containing mRNAs in these cells.

To genetically interrogate the IRPs' role in regulating CD71 of NK cells—and thus their proliferation—the inventors went on to utilize the NK cell line, NK92. In these cells, IRP1 and IRP2 levels were each selectively reduced using an siRNA approach (FIG. 7C-D). Notably, no consistent change in CD71 protein expression was observed upon silencing of IRP1, whereas CD71 abundance significantly dropped in IRP2-silenced cells (FIG. 7E). Accordingly, increased ferritin heavy chain expression was also found when silencing IRP2, but not of IRP1 (FIG. 7F). To ascertain robustness of this finding, the inventors repeated these experiments using a second NK cell line (NKL cells). Similar to NK92 cells, silencing of IRP2 significantly lowered CD71 and increased ferritin heavy chain expression also in this cell line (FIG. 7 G-K). Lastly, knocking out IRP2 using CRISPR/Cas9 technology (FIG. 7L), also reduced CD71 expression among NK92 cells, which directly translated into reduced proliferation rates (FIG. 7M). These data thus recapitulated the findings made in CE vs. NV NK cells through genetic manipulation, and established the IRE/IRP regulatory axis—and specifically IRE/IRP2—is an important system regulating proliferation in NK cells/NK cell lines through governing expression of CD71.

### Example 10: Enforced IRP Expression is a Molecular Module Also Supporting T Cell Proliferation

Adoptive cell therapy using engineered chimeric antigen receptor (CAR) T cells is a promising approach for control of various malignancies, in particular the treatment of hematologic malignancies. However, not all patients respond to the CAR T cell therapy, some relapse—and treatment of solid cancer remains uniquely challenging. Building on the results from the NK cell studies, the inventors reasoned that genetically enforcing pseudo iron deficiency may specifically improve activation-driven (i.e. context dependent) proliferation—and thus the therapeutic potential—also of (CAR) T cells. To begin to examine the role of IRPs in regulating expression of CD71 and interlinked proliferation in T cells, the inventors first suppressed abundance of IRP1 and IRP2 in Jurkat T cells, using siRNA technology (FIG. 8A-B). Similar to NK cell lines, reduction of IRP2 was the dominant factor in reducing expression of CD71 and increasing protein abundance of the ferritin heavy chain (FIG. 8 C-D). Inversely, lentiviral overexpression of IRP2 (LV-IREB2) in IRP2 knockout (ko) Jurkat cells increased CD71 expression as compared to cells transduced with a control vector coding for mCherry (LV-mCherry) (FIG. 8 E-F, top and lower left panels). LV-IREB2 dependent increased cell surface expression of CD71 was associated with more rapid Jurkat T cell proliferation (FIG. 8F, lower right panel). Importantly, expression of CD71 was regulated by lentiviral transduction of LV-IREB2 also in human primary CD4⁺ and CD8⁺ T cells (FIG. 8G-H).

Encouraged by these observations, the inventors went on to test how pseudo iron deficiency—enforced through the expression of IREB2—affected regulation of CD71 and interlinked proliferation in primary human T cells expressing a CAR. For these proof of concept experiments, a CAR T cell model targeting the human prostate-specific membrane antigen (hPSMA) was used. CD4⁺ T cells were either lentivirally transduced with a vector encoding for the CAR (CAR), or both the CAR and IRP2 (CAR IREB2). Overexpression of IRP2 in CAR_IREB2 T cells was confirmed by Western blot analyses (FIG. 81). When assessed under non-activating conditions, overexpression of IRP2 did not affect cell-surface expression of CD71 (FIG. 8J, upper panels). However, expression of CD71 was consistently higher on CAR_IREB2 when compared to CAR T cells upon crosslinking CARs with an α-Fab antibody (FIG. 8J, lower panels). CAR_IREB2 T cells did not spontaneously proliferate (FIG. 8K, upper panel), yet increased IRP2-driven, and hence strictly activation-dependent expression of CD71 was sufficient to drive superior proliferation (FIG. 8K, lower panel). Taken together, these data demonstrate that IRP2 is regulating expression of CD71 and interlinked cell proliferation also in T cells, and specifically CAR T cells. Importantly, inducing pseudo iron deficiency through overexpression of IRP2 in CAR T cells enhanced proliferation in a strictly activation, i.e. context dependent manner.

1. A lymphocyte comprising a synthetic polynucleotide encoding at least one iron regulatory protein (IRP), wherein the at least one iron regulatory protein is IRP1 (SEQ ID NO: 1) and/or IRP2 (SEQ ID NO: 2-6).

2. The lymphocyte according to claim 1, wherein the synthetic polynucleotide encodes IRP2 as set forth in SEQ ID NO:2.

3. The lymphocyte according to claim 1, wherein the lymphocyte is a T cell or a natural killer (NK) cell.

4. The lymphocyte according to claim 3, wherein the lymphocyte is a tumor infiltrating lymphocyte, a modified T cell or a virus specific T cell.

5. The lymphocyte according to claim 1, wherein the at least one iron regulatory protein is constitutively expressed.

6. The lymphocyte according to claim 1, wherein the synthetic polynucleotide encoding the at least one iron regulatory protein is under control of a constitutive promoter.

7. The lymphocyte according to claim 6, wherein the constitutive promoter is an EF-1α promoter.

8. The lymphocyte according to claim 1, wherein the lymphocyte further comprises a chimeric antigen receptor (CAR).

9. The lymphocyte according to claim 8, wherein the CAR comprises an antigen binding domain, a transmembrane domain, a co-stimulatory signaling region and a signaling domain.

10. The lymphocyte according to claim 9, wherein the antigen binding domain is an antibody or an antigen-binding fragment thereof.

11. The lymphocyte according to claim 10, wherein the antigen-binding fragment is a Fab or an scFv.

12. The lymphocyte according to claim 9, wherein the antigen binding domain specifically binds a tumor antigen or a viral antigen.

13. The lymphocyte according to claim 12, wherein the tumor antigen is present on the surface of cells of a target cell population or tissue.

14. The lymphocyte according to claim 8, wherein the CAR is encoded by a polynucleotide, wherein the polynucleotide encoding the CAR is transcriptionally linked to the synthetic polynucleotide encoding IRP1 and/or IRP2.

15. The lymphocyte according to claim 14, wherein the polynucleotide encoding the CAR and the synthetic polynucleotide encoding IRP1 and/or IRP2 are linked by a polynucleotide encoding a self-cleaving peptide.

16. The lymphocyte according to claim 15, wherein the self-cleaving peptide is a 2A self-cleaving peptide.

17. The lymphocyte according to claim 15, wherein the self-cleaving peptide is T2A.

18. A viral vector comprising at least one polynucleotide encoding IRP1 (SEQ ID NO: 1) and/or IRP2 (SEQ ID NO: 2-6).

19. The viral vector according to claim 18, wherein the viral vector comprises a polynucleotide encoding IRP2 as set forth in SEQ ID NO:2.

20. The viral vector according to claim 18, wherein the viral vector is derived from a lentivirus, an adeno-associated virus (AAV), an adenovirus, a herpes simplex virus, a retrovirus, an alphavirus, a flavivirus, a rhabdovirus, a measles virus, a Newcastle disease virus or a poxvirus.

21. (canceled)

22. The viral vector according to claim 18, wherein the at least one polynucleotide encoding IRP1 and/or IRP2 is under control of a constitutive promoter.

23. The viral vector according to claim 22, wherein the constitutive promoter is an EF-1α promoter.

24. The viral vector according to claim 18, wherein the viral vector comprises a further polynucleotide encoding a CAR.

25. The viral vector according to claim 24, wherein the polynucleotide encoding the CAR is transcriptionally linked to the polynucleotide encoding IRP1 and/or IRP2.

26. The viral vector according to claim 25, wherein the polynucleotide encoding the CAR and the polynucleotide encoding IRP1 and/or TRP2 are linked by a polynucleotide encoding a self-cleaving peptide.

27. The viral vector according to claim 26, wherein the self-cleaving peptide is a 2A self-cleaving peptide.

28. The viral vector according to claim 26, wherein the self-cleaving peptide is T2A.

29. A pharmaceutical composition comprising the lymphocyte according to claim 1 and a pharmaceutically acceptable carrier.

30-35. (canceled)

36. A method for treating a subject having cancer or for preventing and/or treating a viral infection in a subject, the method comprising administering to the subject a therapeutically effective amount of the lymphocyte according to claim 1.

37. The method according to claim 36, wherein the cancer is a hematologic cancer or a solid tumor, in particular wherein the hematologic cancer is acute lymphoblastic leukemia, diffuse large B-cell lymphoma, Hodgkin's lymphoma, acute myeloid leukemia or multiple myeloma and wherein the solid tumor is colon cancer, breast cancer, pancreatic cancer, ovarian cancer, hepatocellular carcinoma, lung cancer, neuroblastoma, glioblastoma or sarcoma.

38. The method according to claim 36, wherein the viral infection is caused by human immunodeficiency virus (HIV), adenoviruses, polyomaviruses, influenza virus or human herpesvirus, in particular wherein the human herpesvirus is cytomegalovirus (CMV), Epstein-Barr virus (EBV), herpes simplex virus (HSV), Varizella-Zoster virus (VZV) or human herpesvirus 8 (HHV8).

39. A method for producing the lymphocyte according to claim 1, the method comprising the steps of:
a) providing a lymphocyte obtained from a subject;
b) introducing a synthetic polynucleotide encoding at least one iron regulatory protein into the lymphocyte of step (a), wherein the iron regulatory protein is IRP1 (SEQ ID NO:1) and/or IRP2 (SEQ ID NO:2-6); and
c) expressing the at least one iron regulatory protein encoded by the synthetic polynucleotide that has been introduced into the lymphocyte in step (b).

40. The method according to claim 39, wherein a second synthetic polynucleotide encoding a chimeric antigen receptor (CAR) is introduced into the lymphocyte in step (b).

41. The method according to claim 40, wherein the synthetic polynucleotide encoding the CAR is:
(a) combined with the synthetic polynucleotide encoding IRP1 and/or IRP2 or
(b) transcriptionally linked to the synthetic polynucleotide encoding IRP1 and/or IRP2.

42. (canceled)

43. The method according to claim 40, wherein the synthetic polynucleotide encoding the CAR and the polynucleotide encoding IRP1 and/or IRP2 are linked by a polynucleotide encoding a self-cleaving peptide.

44. The method according to claim 43, wherein the self-cleaving peptide is a 2A self-cleaving peptide.

45. The method according to claim 43, wherein the self-cleaving peptide is T2A.

46. The method according to claim 39, wherein the one or more synthetic polynucleotide is introduced into the lymphocyte by viral transduction.

47. (canceled)

48. The method according to claim 39, wherein the lymphocyte is activated before or after the one or more synthetic polynucleotide is introduced into the lymphocyte.
